(19) United States
(12) Patent Application Publication
(10) Pub. No.: US 2025/0367332 A1
(43) Pub. Date: Dec. 4, 2025

(54) PSMA-TARGETED RADIOACTIVE METAL COMPLEX CONTAINING NITROAROMATIC HETEROCYCLIC GROUP AND PREPARATION METHOD

(71) Applicant: BEIJING NORMAL UNIVERSITY, Beijing (CN)

(72) Inventors: Lin ZHU, Beijing (CN); Wenbin JIN, Beijing (CN); Yang LUO, Beijing (CN); Ran WANG, Beijing (CN); Hank F KONG, Philadelphia, PA (US)

(21) Appl. No.: 19/299,324

(22) Filed: Aug. 13, 2025

Related U.S. Application Data
(63) Continuation of application No. PCT/CN2023/075960, filed on Feb. 14, 2023.

(51) Int. Cl. A61K 51/04

(52) U.S. Cl. CPC A61K 51/0497; A61K 2121/00

(57) ABSTRACT

A PSMA-targeted radioactive metal complex containing a nitroaromatic heterocyclic and nitrophenyl group and its preparation method. The complex can be represented by either of Formula I and Formula II:

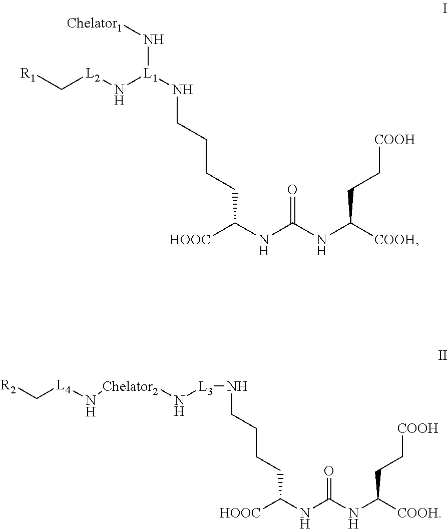

The R and R groups are nitroaromatic heterocyclic groups; L is a linker group between the R group and L; L is a linker group between the chelator and the PSMA-targeted structure; L is a linker group between the R group and L; L is a linker group between the chelator and the PSMA-targeted structure; Chelator₁ and Chelator₂ are chelators or chelating structures.

### CROSS-REFERENCE TO RELATED APPLICATIONS

This application is a continuation of International Patent Application No. PCT/CN2023/075960 with a filing date of Feb. 14, 2023, designating the United States, now pending. The content of the aforementioned applications, including any intervening amendments thereto, are incorporated herein by reference.

### TECHNICAL FIELD

The present disclosure relates to the field of radioactive drugs and medical imaging technologies, specifically to a PSMA-targeted radioactive metal complex containing a nitroaromatic heterocyclic group and its preparation method.

### BACKGROUND

Prostate cancer (PCa) is one of the most common malignant tumors in the male genitourinary system. Most patients can be successfully treated with radical prostatectomy in the early stages, but prostate cancer is highly prone to systemic metastasis. By the time of diagnosis, 60%-80% of patients have already progressed to advanced stages with metastasis, making early diagnosis critically important. Conventional diagnostic methods include screening based on serum prostate-specific antigen (PSA) levels, transrectal prostate ultrasound, pelvic MRI, and prostate biopsy. However, these conventional approaches are invasive, lack certainty, and fail to achieve early and precise diagnosis. In contrast, modern nuclear medicine offers precision and trace-level sensitivity. Radionuclide-labeled probe molecules can specifically recognize and accumulate at lesion sites. By adjusting the type of radionuclide used, they enable synergistic diagnosis and treatment of tumors, facilitating early and accurate cancer management while providing personalized therapeutic regimens for patients.

Prostate-specific membrane antigen (PSMA) is currently an ideal target for prostate cancer diagnosis and treatment. It is selectively overexpressed on the surface of prostate cancer cells, in lymph node metastases, and bone metastases, with expression levels 100-1,000 times higher in cancer cells than in normal tissues. PSMA is expressed in nearly all stages of prostate cancer, and its expression level significantly correlates with disease progression, providing reliable evidence for tumor grading and pathological staging. Additionally, PSMA's transmembrane structure allows it to internalize after binding to targeted molecules, facilitating high intracellular concentrations of targeted drugs—a highly attractive feature for tumor-targeted therapy.

The Glu-Urea-Lys (GUL) structure is the key unit of PSMA-targeting agents. Multiple PSMA-targeted molecular probes based on the GUL structure have been developed. Among these, [⁶⁸Ga]Ga-HBED-CC-PSMA-11 ([⁶⁸Ga]Ga-PSMA-11) is the most widely used small-molecule PET probe for PSMA-targeted imaging of prostate cancer. It was approved by the U.S. Food and Drug Administration (FDA) on Dec. 1, 2020. [⁶⁸Ga]Ga-HBED-CC-PSMA-11 exhibits rapid and efficient labeling, high affinity for PSMA-positive cells in vitro, and high tumor uptake, low hepatic accumulation, and rapid blood clearance in vivo. However, it is primarily metabolized via the urinary system, resulting in high renal and bladder uptake. [⁶⁸Ga]Ga-HBED-CC-PSMA-093 ([⁶⁸Ga]Ga-PSMA-093), disclosed by Kung, Hank F. et al. in 2017 (Patent Title: UREA-BASED PROSTATE SPECIFIC MEMBRANE ANTIGEN (PSMA) INHIBITORS FOR IMAGING AND THERAPY, No. EP3397968B1), is another promising probe currently in Phase II/III clinical trials. It retains the same pharmacophore and bifunctional chelator as [⁶⁸Ga]Ga-HBED-CC-PSMA-11 but incorporates O-(carboxymethyl)-L-tyrosine as a linker. This modification enhances tumor uptake and addresses the limitations of [⁶⁸Ga]Ga-HBED-CC-PSMA-11, such as high bladder uptake interfering with imaging of primary lesions and suboptimal performance in local recurrence detection.

On the other hand, the bifunctional chelator of HBED-CC-PSMA-11 is HBED-CC, which is unsuitable for therapeutic radionuclides like Lu-177, failing to meet clinical needs for targeted radiotherapy. To address this, Benesova, M. et al. developed a novel targeted radiopharmaceutical for prostate cancer: [¹⁷⁷Lu]Lu-DOTA-PSMA-617 ([¹⁷⁷Lu]Lu-PSMA-617). This compound retains the original GUL pharmacophore but replaces the bifunctional chelator with DOTA, enabling labeling with both ⁶⁸Ga and ¹⁷⁷Lu. The radionuclide-labeled PSMA-617 prolongs tumor uptake and accelerates renal clearance, making it more suitable for clinical targeted therapy of prostate cancer. In March 2022, [¹⁷⁷Lu]Lu-PSMA-617 received FDA approval for treating PSMA-positive prostate cancer.

Prostate cancer is a solid tumor. An ideal PSMA-targeted radiopharmaceutical should achieve high tumor uptake, low non-target tissue uptake, or rapid clearance from non-target tissues after injection. Although [⁶⁸Ga]Ga-PSMA-11 and [¹⁷⁷Lu]Lu-PSMA-617 are FDA-approved, and [⁶⁸Ga]Ga-PSMA-093 is in Phase II/III trials, they exhibit limitations: [⁶⁸Ga]Ga-PSMA-11 and [⁶⁸Ga]Ga-PSMA-093 are limited to PET diagnostic imaging, while [¹⁷⁷Lu]Lu-PSMA-617 requires improved tumor uptake as a therapeutic agent.

Therefore, structural modification of compounds to develop novel PSMA-targeted radioactive metal ligands and their complexes containing nitroaromatic heterocyclic groups is a crucial pathway to optimize in vivo pharmacokinetics, enhance tumor uptake and retention, and discover new radiopharmaceuticals with superior properties for tumor theranostics.

### SUMMARY OF THE DISCLOSURE

One object of the present disclosure is to provide a novel PSMA-targeted radioactive metal ligand containing a nitroaromatic heterocyclic group and a complex thereof, exhibiting high affinity and specificity for PSMA. The introduction of the nitroaromatic heterocyclic group enhances the retention of the complex within target tissues and increases tumor uptake of the targeting molecule, making it a promising compound for targeting PSMA receptors.

The above object of the present disclosure is achieved by the following technical solution.

In a first aspect, a PSMA-targeted radioactive metal ligand containing a nitroaromatic heterocyclic group and a complex thereof, represented by Formula I:

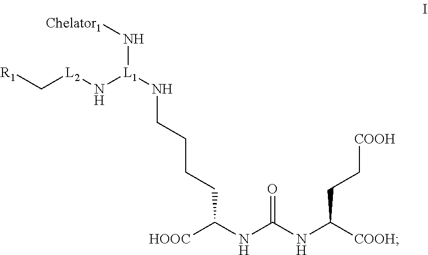

wherein, Chelator₁ is a chelating group or a chelator structure for radionuclides, selected from the group consisting of:

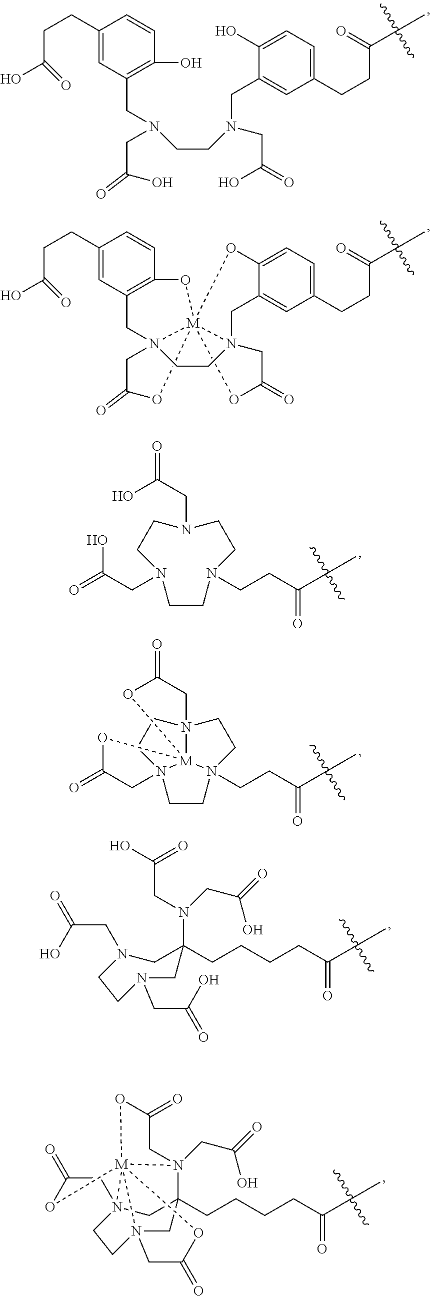

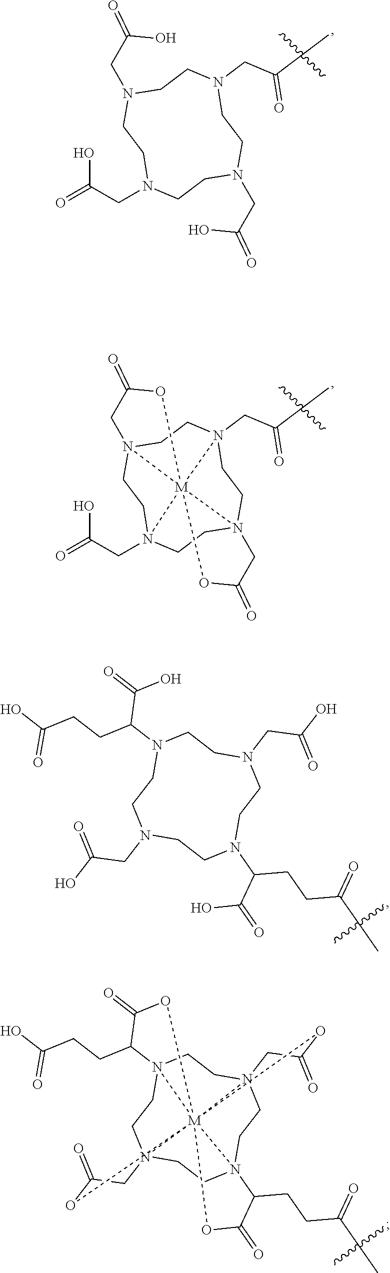

M is selected from the group comprising: ⁶⁸Ga, ¹⁸F—AlF, ¹⁷⁷Lu, ⁹⁰Y, Sc, ²²⁵Ac, ²¹²Pb, ⁶⁴Cu, ¹⁶¹Tb, and ²¹³Bi;
R₁ is a nitroaromatic heterocyclic or nitrophenyl group selected from the group consisting of:

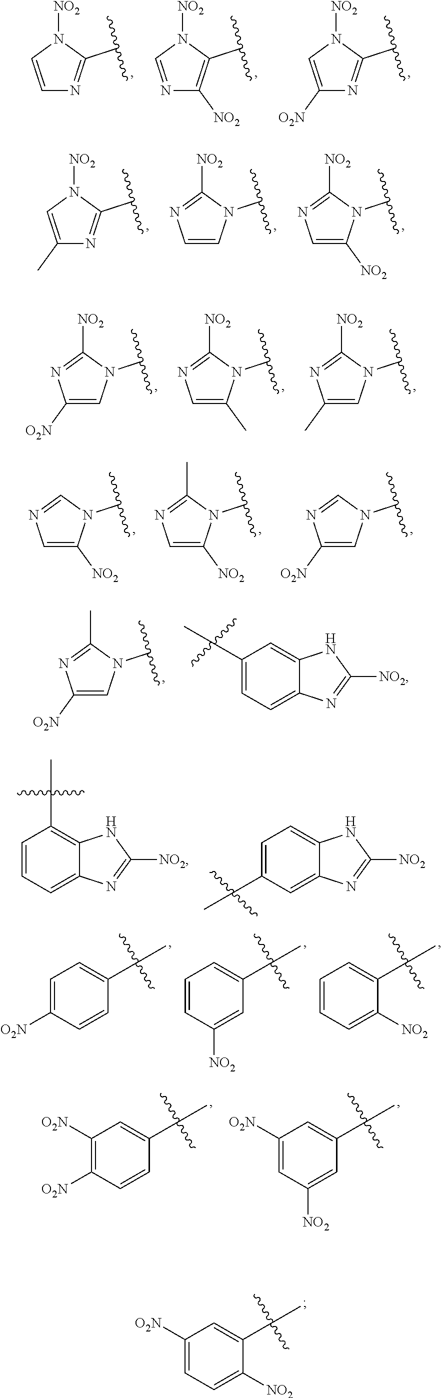

L₁ is a linker group between the Chelator₁ and a PSMA-targeted group, selected from the group consisting of:

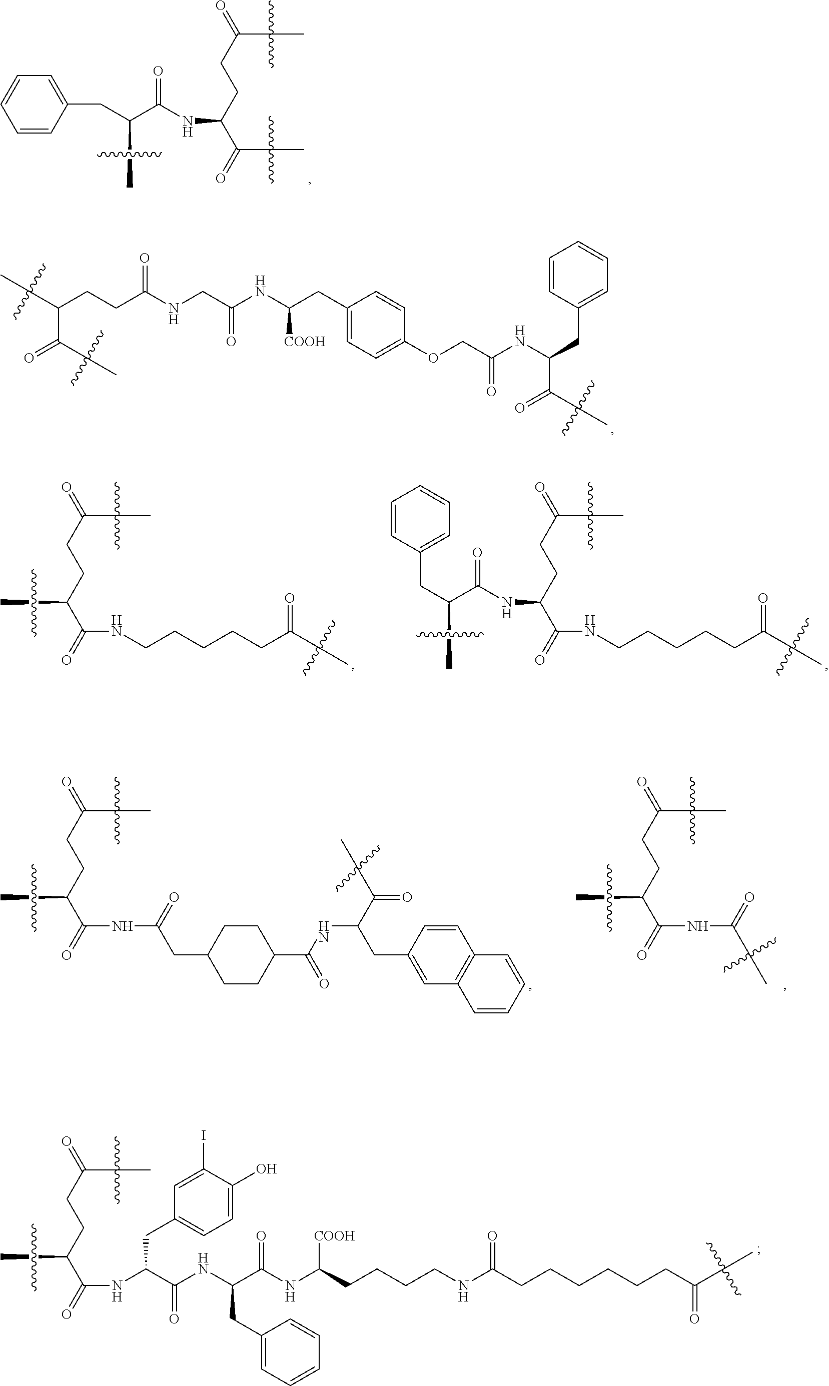

L₂ is a linker group between the Chelator₁ and the R₁, selected from the group consisting of:

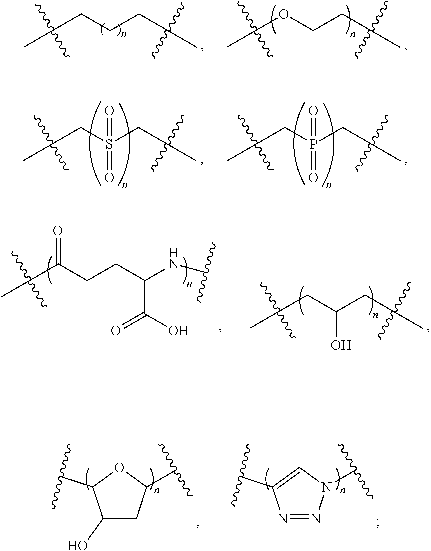

wherein n is an integer in a range from 0 to 6.

In a second aspect, a PSMA-targeted radioactive metal ligand containing a nitroaromatic heterocyclic group and a complex thereof, represented by Formula II:

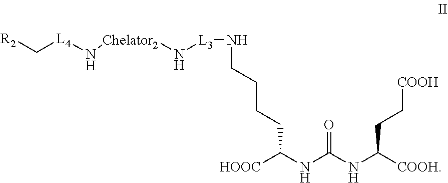

wherein, Chelator₂ is a chelating group or a chelator structure for radionuclides, selected from the group consisting of:

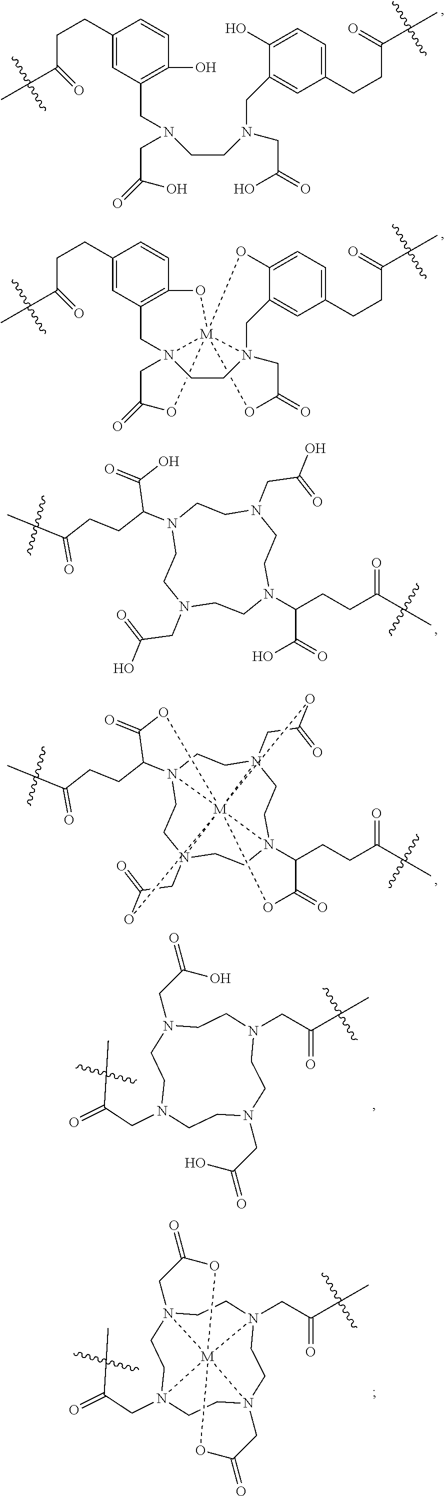

M is selected from the group comprising: ⁶⁸Ga, ¹⁸F—AlF, ¹⁷⁷Lu, ⁹⁰Y, Sc, ²²⁵Ac, ²¹²Pb, ⁶⁴Cu, ⁶¹Tb, and ²¹³Bi;
R₂ is a nitroaromatic heterocyclic or nitrophenyl group selected from the group consisting of:

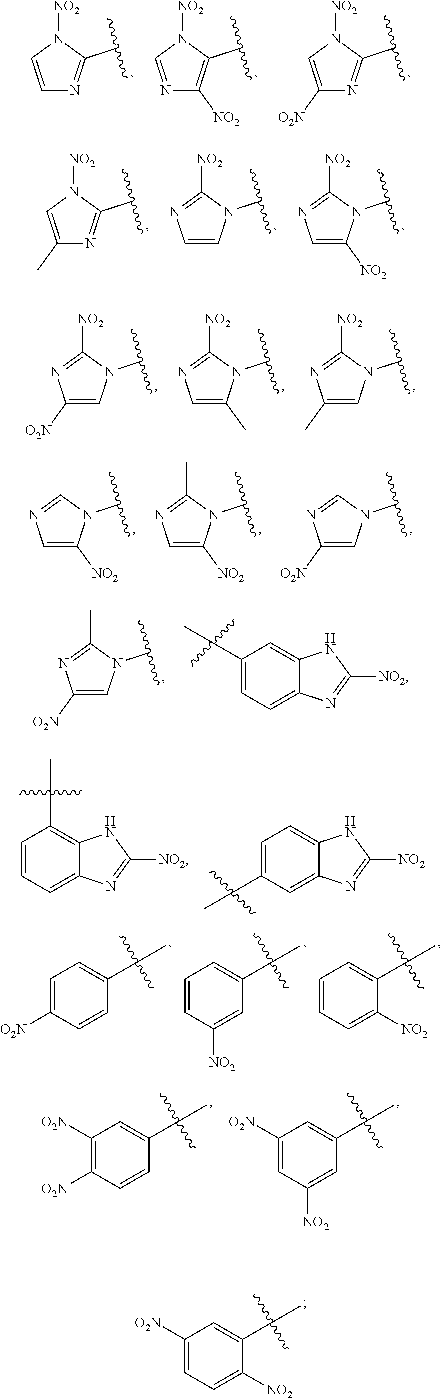

L₃ is a linker group between the Chelator₂ and a PSMA-targeted group, selected from the group consisting of:

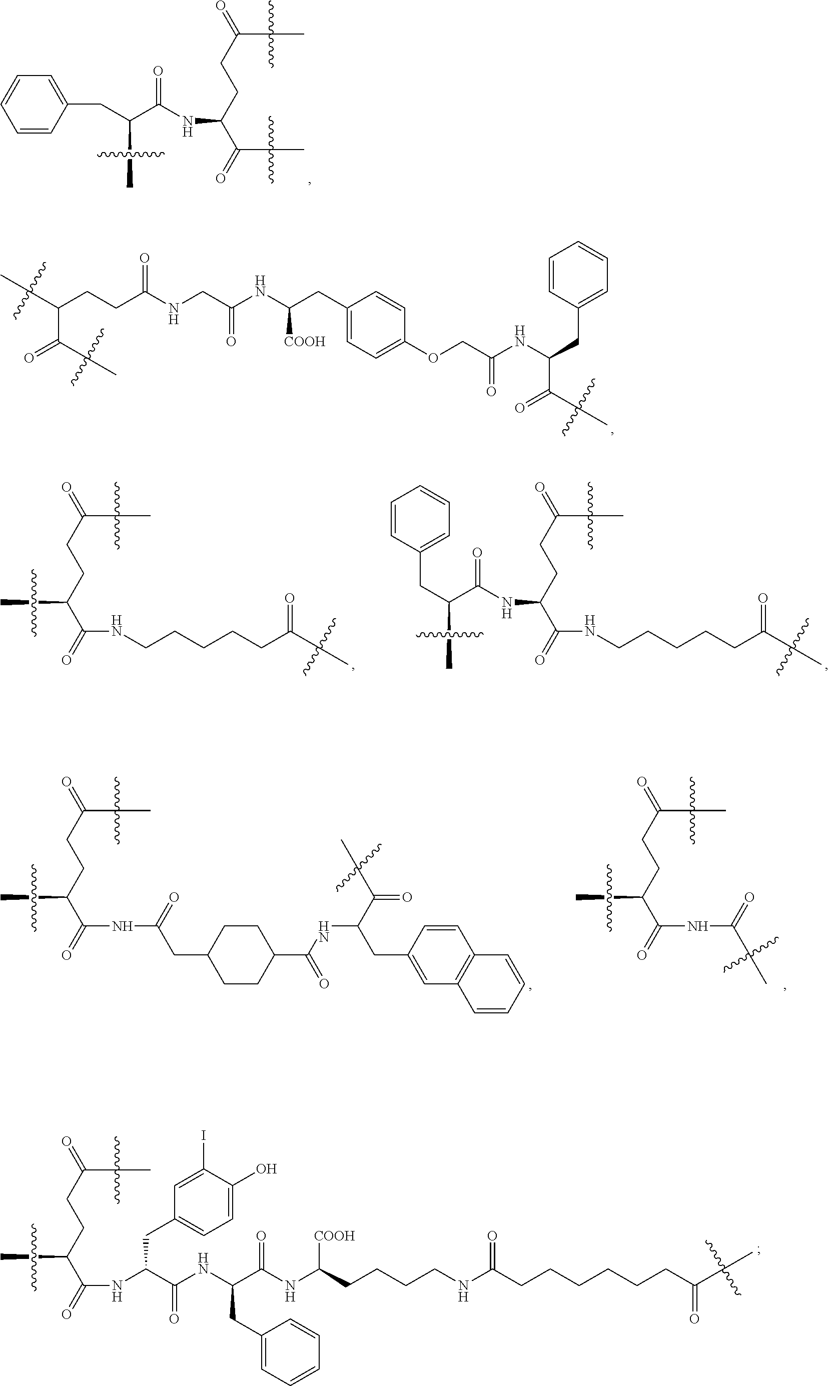

L₄ is a linker group between the Chelator₂ and the R₂, selected from the group consisting of:

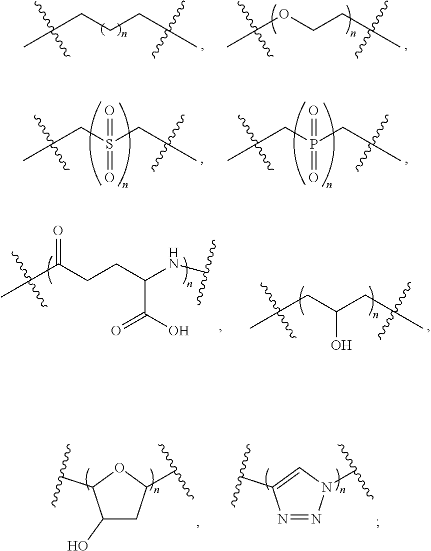

wherein n is an integer in a range from 0 to 6.

Another objective of the present disclosure is to provide a novel preparation method of the PSMA-targeted radioactive metal complexes as above.

The above objective of the present disclosure is achieved by the following technical solutions.

For the complex in the first aspect:
(1) dissolving triphosgene in dichloromethane to form a solution; slowly adding a solution of N(ε)-carbobenzyloxy-L-lysine tert-butyl ester hydrochloride (H-Lys(Z)-Ot-Bu·HCl) and triethylamine in dichloromethane dropwise to the solution, and then slowly adding a solution of L-glutamic acid di-tert-butyl ester hydrochloride and triethylamine in dichloromethane dropwise to form a reaction mixture; stirring the reaction mixture at room temperature, distilling under reduced pressure, and purifying by silica gel column chromatography where a volume ratio of petroleum ether/ethyl acetate is 1/1 to obtain a colorless oily product; dissolving the colorless oily product in tetrahydrofuran, adding 10% Pd/C, and stirring under H atmosphere at room temperature to form a reaction solution; filtering the reaction solution through Celite, and performing evaporation under reduced pressure to remove solvent to obtain brown oily compound Lys(t-Bu)-CO-Glu(t-Bu); dissolving Lys(t-Bu)-CO-Glu(t-Bu) and Cbz-L-NH in anhydrous DMF, and adding 2-(7-azabenzotriazol-1-yl)-N,N,N′,N′-tetramethyluronium hexafluorophosphate (HATU) and N,N′-diisopropylethylamine (DIPEA) under an ice bath for reaction at room temperature overnight to obtain a reaction product; isolating and purifying the reaction product to obtain pale-yellow oily compound; dissolving the pale-yellow oily compound in methanol, adding Pd/C powder for reduction reaction under H atmosphere overnight to obtain NH-L-Lys(t-Bu)-CO-Glu(t-Bu);
(2) dissolving (S)-4-(((9H-fluoren-9-yl)methoxy)carbonyl)amino)-5-(tert-butoxy)-5-oxopentanoic acid in anhydrous DMF, and adding HATU and DIPEA under an ice bath, stirring, then adding R-L-NH to obtain a mixture; stirring the mixture at room temperature overnight to obtain another reaction mixture, and washing the reaction mixture with ethyl acetate and saturated brine; collecting organic phase and drying the organic phase with anhydrous Na SO, filtering and removing the anhydrous Na SO, and performing evaporation under reduced pressure; after solvent removal, performing purification by silica gel column chromatography where a volume ratio of dichloromethane/methanol/ammonia water is 25/1/0.1 to obtain a pale-yellow solid product; dissolving the pale-yellow solid product in trifluoroacetic acid, stirring at room temperature, distilling under reduced pressure to obtain pale-yellow solid compound; dissolving the pale-yellow solid compound in anhydrous DMF, adding HATU and DIPEA under an ice bath, stirring, and adding a solution of NH-L-Lys(t-Bu)-CO-Glu(t-Bu) in anhydrous DMF; leaving for reaction at room temperature overnight to obtain another reaction product, washing the reaction product with ethyl acetate and saturated brine, and collecting organic phase and drying the organic phase with anhydrous Na SO, then filtering and removing the anhydrous Na SO to obtain filtrate; performing evaporation on the filtrate under reduced pressure and removing solvent to obtain residue; purifying the residue by flash chromatography where a volume ratio of dichloromethane/methanol/ammonia water is 15/1/0.1 to obtain another pale-yellow solid compound, dissolving the pale-yellow solid compound in dichloromethane, adding diethylamine dropwise, stirring at room temperature, and performing evaporation under reduced pressure to remove solvent to obtain R-L-NH-L-Lys (t-Bu)-CO-Glu (t-Bu);

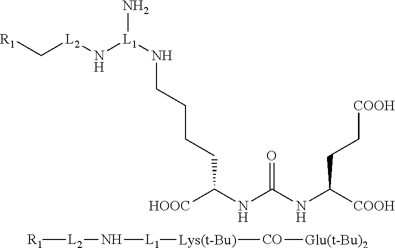

(3) dissolving chelator HBED-CC, AAZTA, DOTA, or NOTA in anhydrous DMF, and adding HATU and DIPEA under an ice bath, stirring, then adding the R-L-NH-L-Lys(t-Bu)-CO-Glu(t-Bu) from step (2); stirring at room temperature overnight, purifying by silica gel column chromatography to obtain pale-yellow oily liquid R-L-NH-L (chelator1)-Lys(t-Bu)-CO-Glu(t-Bu); dissolving the pale-yellow oily liquid in trifluoroacetic acid, stirring at room temperature, distilling under reduced pressure to remove solvent, and purifying by semi-preparative HPLC to obtain the R-L-NH-L (chelator1)-Lys-CO-Glu represented by the Formula I-1;
(4) dissolving the R-L-NH-L (chelator₁)-Lys-CO-Glu from step (3) in sodium acetate buffer; adding a solution of [⁶⁸Ga]GaCl₃ or [¹⁷⁷Lu]LuCl₃ containing radionuclides; and reacting under heating for 5-15 minutes to obtain the radioactive metal complex represented by the Formula I-2.

For the complex in the second aspect:
(1) dissolving triphosgene in dichloromethane to form a solution; slowly adding a solution of N(ε)-carbobenzyloxy-L-lysine tert-butyl ester hydrochloride (H-Lys(Z)-Ot-Bu·HCl) and triethylamine in dichloromethane dropwise to the solution, and then slowly adding a solution of L-glutamic acid di-tert-butyl ester hydrochloride and triethylamine in dichloromethane dropwise to form a reaction mixture; stirring the reaction mixture at room temperature, distilling under reduced pressure, and purifying by silica gel column chromatography where a volume ratio of petroleum ether/ethyl acetate is 1/1 to obtain a colorless oily product; dissolving the colorless oily product in tetrahydrofuran, adding 10% Pd/C, and stirring under H atmosphere at room temperature to form a reaction solution; filtering the reaction solution through Celite, and performing evaporation under reduced pressure to remove solvent to obtain brown oily compound Lys(t-Bu)-CO-Glu(t-Bu); dissolving Lys(t-Bu)-CO-Glu(t-Bu) and Cbz-L₃-NH in anhydrous DMF, and adding 2-(7-azabenzotriazol-1-yl)-N,N,N′,N′-tetramethyluronium hexafluorophosphate (HATU) and N,N′-diisopropylethylamine (DIPEA) under an ice bath for reaction at room temperature overnight to obtain a reaction product; isolating and purifying the reaction product to obtain pale-yellow oily compound; dissolving the pale-yellow oily compound in methanol, adding Pd/C powder for reduction reaction under H atmosphere overnight to obtain NH-L₃-Lys (t-Bu)-CO-Glu (t-Bu);
(2) dissolving chelator HBED-CC, AAZTA, DOTA, or NOTA in anhydrous DMF, and adding HATU and DIPEA under an ice bath, stirring, then adding R-L-NH; stirting at room temperature overnight, washing with ethyl acetate and saturated brine; collecting organic phase and drying the organic phase with anhydrous Na SO, filtering and removing the anhydrous Na SO, and performing evaporation under reduced pressure; after solvent removal, performing purification by silica gel column chromatography where a volume ratio of dichloromethane/methanol/ammonia water is 90/10/0.1 to obtain pale-yellow solid R-L-NH-chelator₂; dissolving the pale-yellow solid in anhydrous DMF, adding HATU and DIPEA under an ice bath, stirring, adding the NH-L-Lys(t-Bu)-CO-Glu(t-Bu) from step (1), and further adding R-L-NH; stirring at room temperature overnight to obtain another reaction product, washing the reaction product with ethyl acetate and saturated brine, and collecting organic phase and drying the organic phase with anhydrous Na SO, then filtering and removing the anhydrous Na SO to obtain filtrate; performing evaporation on the filtrate under reduced pressure and removing solvent to obtain residue; purifying the residue by silica gel column chromatography where a volume ratio of dichloromethane/methanol/ammonia water is 90/10/0.1 to obtain oily liquid R-L-NH-chelator₂-NH-L-Lys (t-Bu)-CO-Glu(t-Bu); dissolving the oily liquid in trifluoroacetic acid, stirring at room temperature, distilling under reduced pressure to remove solvent, and purifying by semi-preparative HPLC to obtain the R-L-NH-chelator₂-NH-L-Lys-CO-Glu represented by the Formula II-1;
(3) adding the following: dissolving the R-L-NH-chelator₂-NH-L-Lys-CO-Glu from step (2) in sodium acetate buffer; adding a solution of [⁶⁸Ga]GaCl₃ or [¹⁷⁷Lu]LuCl₃ containing radionuclides; and reacting under heating for 5-15 minutes to obtain the radioactive metal complex represented by the Formula II-2.

### Beneficial Effects

The novel PSMA-targeted radioactive metal ligand containing a nitroaromatic heterocyclic group of the present disclosure enables labeling with different radionuclides. The prepared radioactive metal complex exhibits high affinity and specificity for PSMA. In addition, the introduction of the nitroaromatic heterocyclic group enhances the retention of the complex within target tissues and increases tumor uptake of the targeting molecule, making it a promising compound for targeting prostate-specific membrane antigen receptors.

The following further illustrates the present disclosure through the accompanying drawings and specific embodiments, but does not imply limitations to the scope of the present disclosure.

### BRIEF DESCRIPTION OF THE DRAWINGS

FIG. 1 shows the radio-HPLC chromatogram of the labeling reaction mixture for [⁶⁸Ga]Ga-HBED-CC-NI-PSMA prepared in Embodiment 1 according to the present disclosure.

FIG. 2 shows the radio-HPLC chromatogram of the labeling reaction mixture for [⁶⁸Ga]Ga-AAZTA-NI-PSMA prepared in Embodiment 2 according to the present disclosure.

FIG. 3 shows the radio-HPLC chromatogram of the labeling reaction mixture for [⁶⁸Ga]Ga-DOTA-NI-PSMA prepared in Embodiment 3 according to the present disclosure.

FIG. 4 shows the radio-HPLC chromatogram of the labeling reaction mixture for [⁶⁸Ga]Ga-HBED-CC-NI-PSMA-11 prepared in Embodiment 4 according to the present disclosure.

FIG. 5 shows the radio-HPLC chromatogram of the labeling reaction mixture for [⁶⁸Ga]Ga-AAZTA-NI-PSMA-11 prepared in Embodiment 5 according to the present disclosure.

FIG. 6 shows the radio-HPLC chromatogram of the labeling reaction mixture for [⁶⁸Ga]Ga-DOTA-NI-PSMA-11 prepared in Embodiment 6 according to the present disclosure.

FIG. 7 shows the radio-HPLC chromatogram of the labeling reaction mixture for [⁶⁸Ga]Ga-AAZTA-NI-PSMA-093 prepared in Embodiment 7 according to the present disclosure.

FIG. 8 shows the radio-HPLC chromatogram of the labeling reaction mixture for [⁶⁸Ga]Ga-NI-HBED-CC-PSMA-11 prepared in Embodiment 8 according to the present disclosure.

FIG. 9 shows the radio-HPLC chromatogram of the labeling reaction mixture for [⁶⁸Ga]Ga-NI-DOTAGA2-PSMA-11 prepared in Embodiment 9 according to the present disclosure.

FIG. 10 shows the radio-HPLC chromatogram of the labeling reaction mixture for [⁶⁸Ga]Ga-NI-HBED-CC-PSMA-093 prepared in Embodiment 10 according to the present disclosure.

FIG. 11 shows the radio-HPLC chromatogram of the labeling reaction mixture for [⁶⁸Ga]Ga-NI-DOTAGA2-PSMA-093 prepared in Embodiment 11 according to the present disclosure.

FIG. 12 shows the uptake-time profile (n=3) of [⁶⁸Ga]Ga-AAZTA-NI-PSMA, [⁶⁸Ga]Ga-AAZTA-NI-PSMA-11, and [⁶⁸Ga]Ga-HBED-CC-PSMA-11 in in vitro 22RV1-FOLH1-oe cells in Application Embodiment 1 according to the present disclosure.

FIG. 13 shows the specific binding analysis (n=3) of [⁶⁸Ga]Ga-AAZTA-NI-PSMA, [⁶⁸Ga]Ga-AAZTA-NI-PSMA-11, and [⁶⁸Ga]Ga-HBED-CC-PSMA-11 to prostate-specific membrane antigen receptors in in vitro 22RV1-FOLH1-oe cells in Application Embodiment 1 according to the present disclosure.

FIG. 14 shows the uptake-time profile (n=3) of [⁶⁸Ga]Ga-AAZTA-NI-PSMA, [⁶⁸Ga]Ga-DOTA-NI-PSMA, [⁶⁸Ga]Ga-HBED-CC-NI-PSMA, and [⁶⁸Ga]Ga-HBED-CC-PSMA-11 in in vitro 22RV1-FOLH1-oe cells in Application Embodiment 2 according to the present disclosure.

FIG. 15 shows the specific binding analysis (n=3) of [⁶⁸Ga]Ga-AAZTA-NI-PSMA, [⁶⁸Ga]Ga-DOTA-NI-PSMA, [⁶⁸Ga]Ga-HBED-CC-NI-PSMA, and [⁶⁸Ga]Ga-HBED-CC-PSMA-11 to prostate-specific membrane antigen receptors in in vitro 22RV1-FOLH1-oe cells in Application Embodiment 2 according to the present disclosure.

FIG. 16 shows the uptake-time profile (n=3) of [⁶⁸Ga]Ga-AAZTA-NI-PSMA-093 and [⁶⁸Ga]Ga-HBED-CC-PSMA-11 in in vitro 22RV1-FOLH1-oe cells in Application Embodiment 3 according to the present disclosure.

FIG. 17 shows the specific binding analysis (n=3) of [⁶⁸Ga]Ga-AAZTA-NI-PSMA-093 and [⁶⁸Ga]Ga-HBED-CC-PSMA-11 to prostate-specific membrane antigen receptors in in vitro 22RV1-FOLH1-oe cells in Application Embodiment 3 according to the present disclosure.

FIG. 18 shows the uptake-time profile (n=3) of [⁶⁸Ga]Ga-NI-HBED-CC-PSMA-11, [⁶⁸Ga]Ga-HBED-CC-PSMA-11, [⁶⁸Ga]Ga-NI-HBED-CC-PSMA-093, and [⁶⁸Ga]Ga-HBED-CC-PSMA-093 in in vitro 22RV1-FOLH1-oe cells in Application Embodiment 4 according to the present disclosure.

FIG. 19 shows the specific binding analysis (n=3) of [⁶⁸Ga]Ga-NI-HBED-CC-PSMA-11, [⁶⁸Ga]Ga-HBED-CC-PSMA-11, [⁶⁸Ga]Ga-NI-HBED-CC-PSMA-093, and [⁶⁸Ga]Ga-HBED-CC-PSMA-093 to prostate-specific membrane antigen receptors in in vitro 22RV1-FOLH1-oe cells in Application Embodiment 4 according to the present disclosure.

### DETAILED DESCRIPTION

Unless otherwise specified, the raw materials and reagents mentioned in the embodiments of the present disclosure are conventional raw materials and reagents available on the market, the testing methods applied are conventional methods adopted in the art, and the equipment and devices used are conventional equipment and devices in the art.

### Embodiment 1: Preparation of [⁶⁸Ga]Ga-HBED-CC-NI-PSMA

### Step 1: Synthesis of the PSMA-Targeted Radioligand HBED-CC-NI-PSMA Containing a Nitroaromatic Heterocyclic Group.

The synthesis route is as follows.

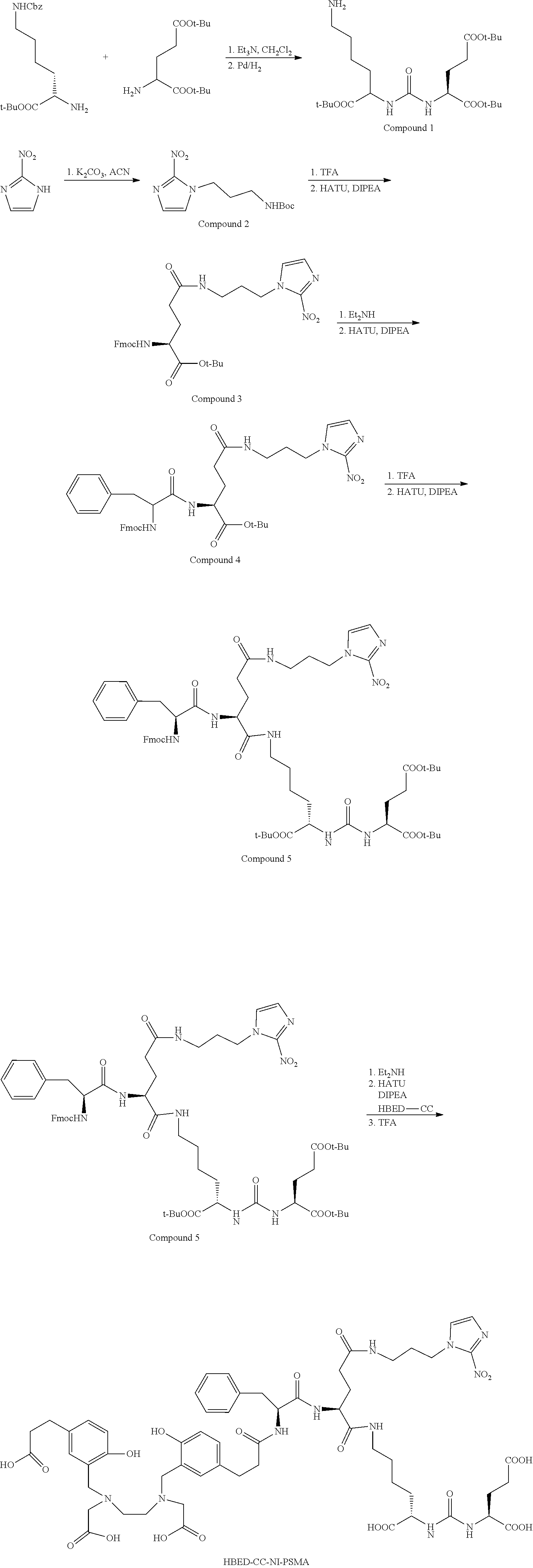

Specifically, the following steps are included.

### (1) Synthesis of Compound 1

Triphosgene (1.20 g, 4.03 mmol) was dissolved in dichloromethane (10 mL) and stirred at −20° C. for 20 minutes. A solution of N(ε)-carbobenzyloxy-L-lysine tert-butyl ester hydrochloride (H-Lys (Z)-Ot-Bu·HCl, 4.47 g, 12.0 mmol) and triethylamine (2.80 mL, 2.04 g, 20.2 mmol) in dichloromethane (75 mL) was slowly added dropwise. Subsequently, a solution of L-glutamic acid di-tert-butyl ester hydrochloride (Glu-Ot-Bu(Ot-Bu)·HCl, 2.90 g, 9.83 mmol) and triethylamine (2.80 mL, 2.04 g, 20.2 mmol) in dichloromethane (50 mL) was slowly added dropwise. The reaction mixture was stirred at room temperature for 18 hours, distilled under reduced pressure, and purified by silica gel column chromatography (petroleum ether/ethyl acetate=1/1, v v) to yield a colorless oily product (3.04 g, 4.89 mmol, yield: 48.9%). The colorless oily product (2.25 g, 3.62 mmol) was dissolved in tetrahydrofuran (20 mL), and 10% Pd/C (192 mg) was added. The mixture was stirred under a hydrogen atmosphere at room temperature for 12 hours. The reaction mixture was filtered through Celite, and the filtrate was concentrated by evaporation under reduced pressure to remove the solvent, yielding a brown oily Compound 1 (1.20 g, 2.46 mmol, yield: 68.0%).

Structural confirmation of Compound 1:

HRMS: m/z calcd for CHNO₇[M+H]⁺: 488.3330, found: 488.3334.

¹H NMR (600 MHz, CDCl) δ: 5.36 (t, 2H, J=7.8 Hz), 4.30 (dq, 2H, J=7.5, 5.3 Hz), 2.65 (t, 2H, J=6.9 Hz), 2.33-2.20 (m, 2H), 2.06-2.00 (m, 2H), 1.85-1.77 (m, 1H), 1.73 (ddt, 1H, J=13.5, 10.4, 5.3 Hz), 1.67 (s, 2H), 1.61-1.53 (m, 1H), 1.42 (d, 18H, J=0.5 Hz), 1.39 (s, 9H), 1.33-1.26 (m, 1H).

### (2) Synthesis of Compound 2

2-Nitroimidazole (1.14 g, 10.09 mmol) was dissolved in anhydrous N,-dimethylformamide (15 mL), followed by addition of anhydrous potassium carbonate (4.89 g, 35.38 mmol). After stirring at room temperature for 30 minutes, N-(3-bromopropyl) carbamic acid tert-butyl ester (3.57 g, 14.99 mmol) in anhydrous N,N-dimethylformamide (10 mL) was added in the above reaction mixture. The reaction mixture was stirred overnight at room temperature, filtered through Celite, and evaporated under reduced pressure to remove most solvent. The residue was washed with ethyl acetate and saturated brine. The organic phase was collected and dried over anhydrous sodium sulfate, and evaporated under reduced pressure to remove the solvent. The residue was purified by silica gel column chromatography (petroleum ether/ethyl acetate, v/v=1/1) to yield a yellow-green oily Compound 2 (2.36 g, 8.74 mmol, yield: 87%).

### Structural Confirmation of Compound 2

HRMS: m/z calcd for CHNO [M+H]⁺: 271.1400, found: 271.1408.

¹H NMR (600 MHz, CDCl) δ: 7.27 (s, 1H), 7.14 (s, 1H), 4.75 (s, 1H), 4.46 (t, J=7.0 Hz, 2H), 3.20 (t, J=6.2 Hz, 2H), 2.08-2.01 (m, 2H), 1.44 (s, 9H).

### (3) Synthesis of Compound 3

Compound 2 (432 mg, 1.60 mmol) was dissolved in trifluoroacetic acid (4 mL) and stirred at room temperature for 30 minutes. After evaporation under reduced pressure to remove the solvent, a white solid intermediate was obtained. (S)-4-((((9H-Fluoren-9-yl) methoxy) carbonyl)amino)-5-(tert-butoxy)-5-oxopentanoic acid (1.5 g, 3.53 mmol) was dissolved in anhydrous N,N-dimethylformamide (10 mL). HATU (1.61 g, 4.23 mmol) and DIPEA (547 mg, 4.23 mmol) were added under an ice bath and stirred for 25 minutes. The white solid intermediate (603 mg, 3.55 mmol) was added, and the reaction mixture was stirred overnight at room temperature. The reaction mixture was washed with ethyl acetate and saturated brine (5×, i.e., five times). The organic phase was collected and dried over anhydrous sodium sulfate and filtered, and the anhydrous sodium sulfate was removed. After evaporation under reduced pressure to remove the solvent, the residue was purified by silica gel column chromatography (dichloromethane/methanol/aq. ammonia, v/v/v=25/1/0.1) to yield a pale-yellow solid Compound 3 (1.5 g, 2.60 mmol, yield: 74%).

### Structural Confirmation of Compound 3

HRMS: m/z calcd for CHN₅O₇[M+H]⁺: 578.2609, found: 578.2609.

¹H NMR (400 MHz, CDCl) δ: 7.76 (d, J=7.6 Hz, 2H), 7.59 (t, J=7.2 Hz, 2H), 7.40 (t, J=7.5 Hz, 2H), 7.35-7.28 (m, 2H), 7.27 (s, 1H), 7.09 (s, 1H), 6.57 (s, 1H), 5.65 (d, J=7.2 Hz, 1H), 4.41 (dd, J=12.1, 7.0 Hz, 4H), 4.21 (t, J=7.0 Hz, 2H), 3.42-3.32 (m, 1H), 3.31-3.21 (m, 1H), 2.35-2.18 (m, 3H), 2.10-1.99 (m, 2H), 1.92-1.81 (m, 1H), 1.47 (s, 9H).

### (4) Synthesis of Compound 4

Compound 3 (602 mg, 1.04 mmol) was dissolved in dichloromethane (10 mL), and diethylamine (2.49 g, 33.98 mmol) was added dropwise. After stirring at room temperature for 3 hours, the solvent was removed by evaporation under reduced pressure to yield a pale-yellow solid intermediate. Fmoc-L-phenylalanine (327 mg, 0.84 mmol) was dissolved in anhydrous N,N-dimethylformamide (3 mL). HATU (386 mg, 1.02 mmol) and DIPEA (218 mg, 1.69 mmol) were added under an ice bath and stirred for 25 minutes. The pale-yellow solid intermediate (300 mg, 0.84 mmol) in anhydrous N,N-dimethylformamide (3 mL) was added, and the above reaction mixture was stirred overnight at room temperature. The reaction mixture was washed with ethyl acetate and saturated brine (5×). The organic phase was collected and dried over anhydrous sodium sulfate and filtered, and the anhydrous sodium sulfate was removed. After evaporation under reduced pressure for concentration, a white solid Compound 4 (362 mg, 0.50 mmol, yield: 60%) is precipitated.

### Structural Confirmation of Compound 4

HRMS: m/z calcd for CHN₆O₈[M+H]⁺: 725.3298, found: 725.3300.

¹H NMR (400 MHz, (CD) SO) δ: 8.42 (d, J=7.3 Hz, 1H), 7.97-7.77 (m, 3H), 7.68 (s, 1H), 7.66-7.56 (m, 3H), 7.44-7.37 (m, 2H), 7.36-7.31 (m, 2H), 7.30-7.23 (m, 4H), 7.22-7.15 (m, 2H), 4.45-4.24 (m, 3H), 4.23-3.97 (m, 4H), 3.05 (dd, J=13.6, 8.7 Hz, 3H), 2.78 (t, J=12.5 Hz, 1H), 2.18 (t, j=7.4 Hz, 2H), 2.05-1.94 (m, 1H), 1.94-1.76 (m, 3H), 1.40 (s, 9H).

### (5) Synthesis of Compound 5

Compound 4 (500 mg, 0.69 mmol) was dissolved in dichloromethane (2 mL), and trifluoroacetic acid (2 mL) was added. After stirring at room temperature for 30 minutes and evaporation under reduced pressure to remove the solvent, an orange-yellow oily intermediate is yielded. The intermediate (358 mg, 0.54 mmol) was dissolved in anhydrous N,N-dimethylformamide (4 mL). HATU (240 mg, 0.63 mmol) and DIPEA (134 mg, 1.04 mmol) were added under an ice bath and stirred for 30 minutes. Compound 1 (276 mg, 0.57 mmol) in anhydrous N A dimethylformamide (3 mL) was added into the above reaction mixture, which was then stirred overnight at room temperature. The reaction mixture was washed with ethyl acetate and saturated brine (5×). The organic phase was collected and dried over anhydrous sodium sulfate and filtered, and the anhydrous sodium sulfate was removed. After evaporation under reduced pressure to remove the solvent, the residue was purified by flash chromatography (dichloromethane/methanol/aq. ammonia, v/v/v=15/1/0.1) to yield a pale-yellow solid Compound 5 (408 mg, 0.36 mmol, yield: 67%).

### Structural Confirmation of Compound 5

HRMS: m/z calcd for C₅₉H₈₀N₉O [M+H]⁺: 1138.5819, found: 1138.5824.

¹H NMR (400 MHz, CDCl) δ: 7.90 (s, 1H), 7.73 (d, J=7.4 Hz, 2H), 7.50 (d, J=7.2 Hz, 2H), 7.37 (t, J=7.3 Hz, 2H), 7.30-7.21 (m, 11H), 7.08 (d, J=12.1 Hz, 1H), 6.97 (s, 1H), 5.79 (s, 1H), 5.29 (s, 1H), 4.54-4.29 (m, 6H), 4.27-4.09 (m, 3H), 3.43-2.90 (m, 6H), 2.30 (s, 4H), 2.18-1.92 (m, 6H), 1.91-1.75 (m, 1H), 1.74-1.54 (m, 2H), 1.46-1.37 (m, 27H), 1.25 (t, J=7.1 Hz, 2H).

### (6) Synthesis of HBED-CC-NI-PSMA

Compound 5 (191 mg, 0.17 mmol) was dissolved in dichloromethane (3 mL), and diethylamine (1 mL) was added. After stirring at room temperature for 3 hours, the solvent was removed by evaporation under reduced pressure to yield a yellow oily intermediate. HBED-CC (39 mg, 0.06 mmol) was dissolved in anhydrous N,N-dimethylformamide (2 mL). HATU (27.4 mg, 0.07 mmol) and DIPEA (15.51 mg, 0.12 mmol) were added under an ice bath and stirred for 30 minutes. The yellow oily intermediate (61.9 mg, 0.07 mmol) in anhydrous N, A-dimethylformamide (2 mL) was added, and the mixture was under reaction overnight at room temperature. The reaction mixture was washed with ethyl acetate and saturated brine (5×). The organic phase was collected and dried over anhydrous sodium sulfate and filtered, and the anhydrous sodium sulfate was removed. After evaporation under reduced pressure to remove the solvent, the residue was purified by flash chromatography (dichloromethane/methanol/aq. ammonia, v/v/v=9/1/0.1) to yield a yellow oily product. The yellow oily product was then deprotected with trifluoroacetic acid to yield crude HBED-CC-NI-PSMA, which was purified by HPLC (Semi-preparative chromatography column: Eclipse XDB-C18, 5 μm, 9.4×250 mm. Separation conditions: Phase A: 0.1% TFA in HO, Phase B: 0.1% TFA in CH CN. gradient: 0-12 min, 5-55% B; 12-13 min, 55-100% B; 13-16 min, 100% B; 16-17 min, 100-5% B; 17-20 min, 5% B. flow rate: 4 mL/min. UV: 280 nm). The target fraction was obtained as a white solid compound HBED-CC-NI-PSMA through freeze-drying.

Structural Confirmation of HBED-CC-NI-PSMA.

HRMS: m/z calcd for C₅₈H₇₇NO [M+H]⁺: 1262.5211, found: 1262.5220.

### Step 2: ⁶⁸Ga Labeling of the PSMA-Targeted Radioligand HBED-CC-NI-PSMA Containing a Nitroaromatic Heterocyclic Group

The ⁶⁸Ga labeling route is as follows.

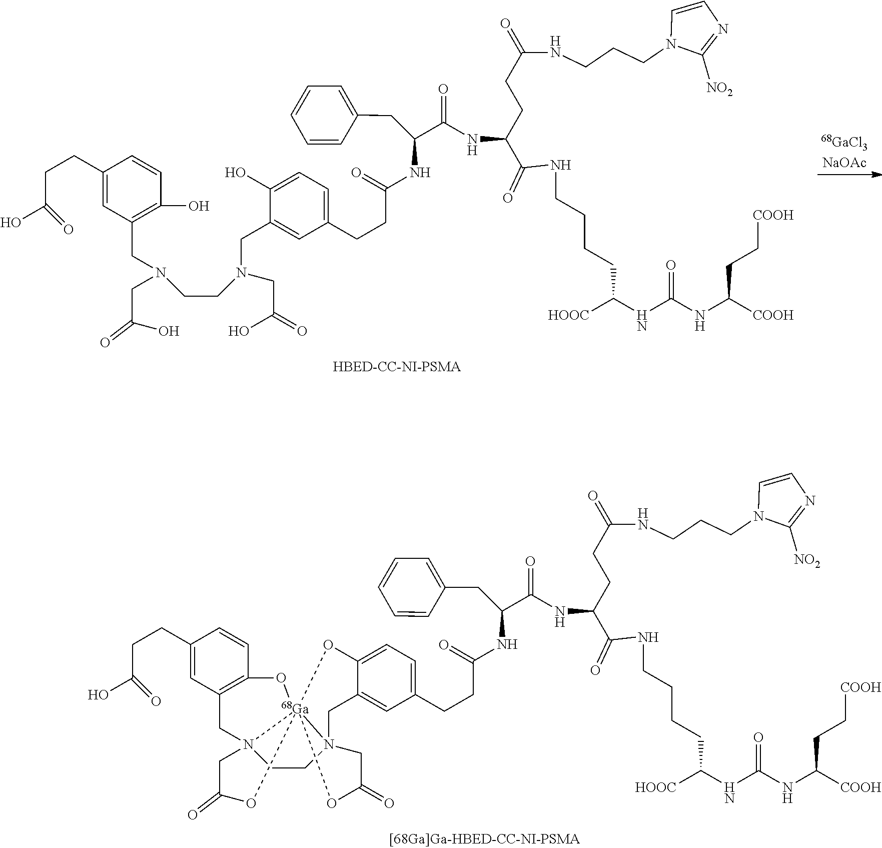

HBED-CC-NI-PSMA obtained in Step 1 was dissolved in dimethyl sulfoxide (DMSO) to prepare a precursor solution (1 μg/μL). 4 μL of the precursor solution was added in a 10 mL vial, and 135 μL of 3 M sodium acetate solution was added. The germanium-gallium generator (iThemba) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 50° C. for 10 minutes, followed by cooling to room temperature. The radiochemical purity was determined by radio-HPLC equipped with a radioactive detector, confirming a radiochemical yield of >98% for [⁶⁸Ga]Ga-HBED-CC-NI-PSMA.

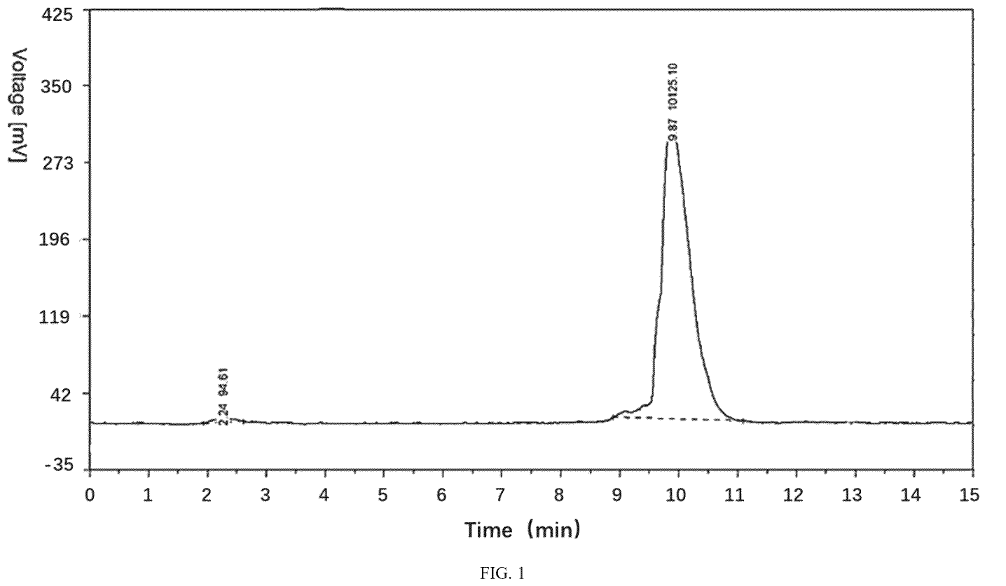

As shown in FIG. 1, FIG. 1 shows the radio-HPLC chromatogram of the labeling reaction mixture for [⁶⁸Ga]Ga-HBED-CC-NI-PSMA prepared in Embodiment 1 according to the present disclosure, which demonstrates a radiochemical purity exceeding 98%.

### Embodiment 2: Preparation of [⁶⁸Ga]Ga-AAZTA-NI-PSMA

### Step 1: Synthesis of the PSMA-Targeted Radioligand AAZTA-NI-PSMA Containing a Nitroaromatic Heterocyclic Group.

The synthesis route is as follows.

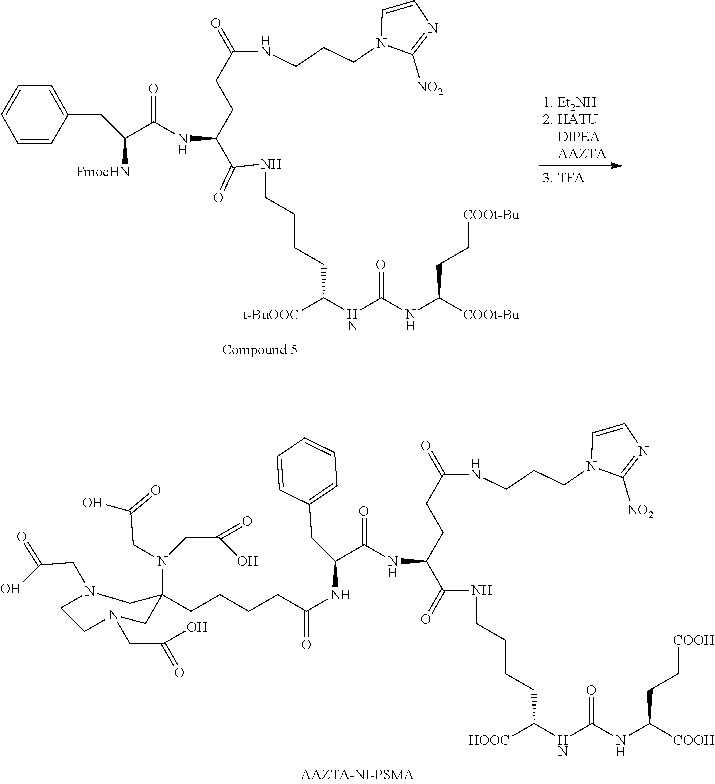

Specifically, the following steps are included.

### Synthesis of AAZTA-NI-PSMA

Compound 5 (191 mg, 0.17 mmol) was dissolved in dichloromethane (3 mL), followed by the addition of diethylamine (1 mL). The mixture was stirred at room temperature for 3 hours. The solvent was removed by evaporation under reduced pressure to yield a yellow oily intermediate. AAZTA (41 mg, 0.06 mmol) was dissolved in anhydrous N,N-dimethylformamide (2 mL). HATU (27.4 mg, 0.07 mmol) and DIPEA (15.51 mg, 0.12 mmol) were added under an ice bath and stirred for 30 minutes. The yellow oily intermediate (61.9 mg, 0.07 mmol) in anhydrous N,N-dimethylformamide (2 mL) was added to the above reaction mixture and stirred overnight at room temperature. The reaction mixture was washed with ethyl acetate and saturated brine (5×). The organic phase was collected and dried over anhydrous sodium sulfate and filtered, and the anhydrous sodium sulfate was removed. After evaporation under reduced pressure to remove the solvent, the residue was purified by flash chromatography (dichloromethane/methanol/aq. ammonia, v/v/v=10/1/0.1) to yield a yellow oily product. The yellow oily product was then deprotected with trifluoroacetic acid to yield crude AAZTA-NI-PSMA, which was purified by HPLC (Semi-preparative chromatography column: Eclipse XDB-C18, 5 μm, 9.4×250 mm. Separation conditions: Phase A: 0.1% TFA in H O, Phase B: 0.1% TFA in CH CN. gradient: 0-16 min, 5-41% B; 16-16.5 min, 41-100% B; 16.5-20 min, 100% B; 20-20.5 min, 100-5% B; 20.5-23 min, 5% B. flow rate: 4 mL/min. UV: 280 nm). The target fraction was obtained as a white solid compound AAZTA-NI-PSMA through freeze-drying.

Structural confirmation of AAZTA-NI-PSMA: Purity: >98% (confirmed by LC-MS).

### Step 2: ⁶⁸Ga Labeling of the PSMA-Targeted Radioligand AAZTA-NI-PSMA Containing a Nitroaromatic Heterocyclic Group

The ⁶⁸Ga labeling route is as follows.

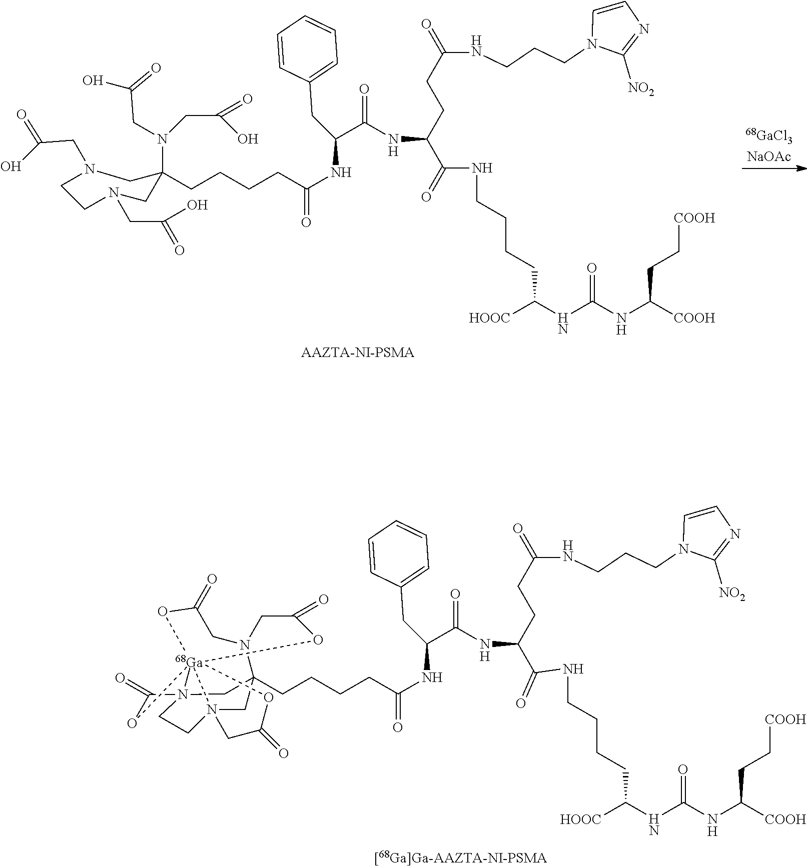

AAZTA-NI-PSMA obtained in Step 1 was dissolved in dimethyl sulfoxide (DMSO) to prepare a precursor solution (1 μg/μL). 10 μL of the precursor solution was added to a 10 mL vial, and 135 μL of 3 M sodium acetate solution was added. The germanium-gallium generator (iThemba) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 50° C. for 10 minutes, followed by cooling to room temperature. The radiochemical purity was determined by radio-HPLC equipped with a radioactive detector, confirming a radiochemical yield of >98% for [⁶⁸Ga]Ga-AAZTA-NI-PSMA.

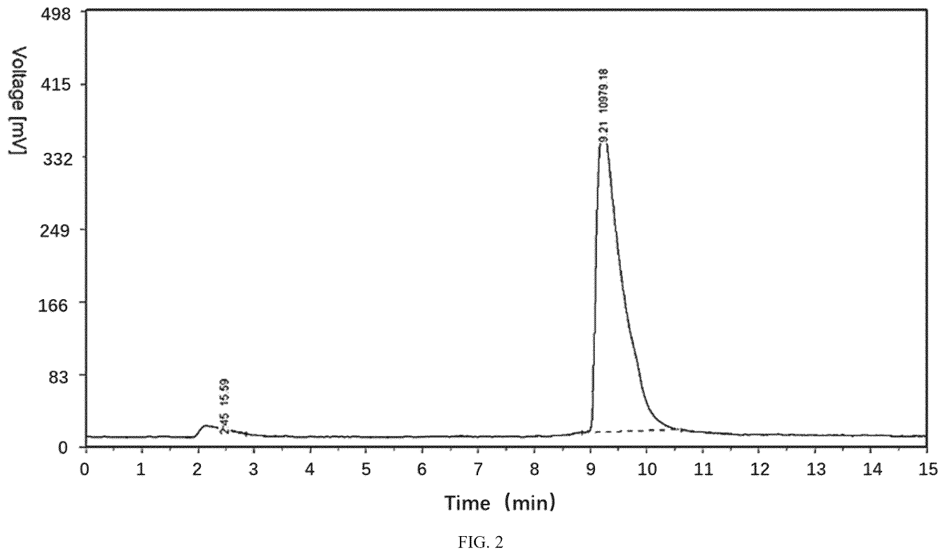

As shown in FIG. 2, FIG. 2 shows the radio-HPLC chromatogram of the labeling reaction mixture for [⁶⁸Ga]Ga-AAZTA-NI-PSMA prepared in Embodiment 2 according to the present disclosure, which demonstrates a radiochemical purity exceeding 98%.

### Embodiment 3: Preparation of [⁶⁸Ga]Ga-DOTA-NI-PSMA

### Step 1: Synthesis of the PSMA-Targeted Radioligand DOTA-NI-PSMA Containing a Nitroaromatic Heterocyclic Group

The synthesis route is as follows.

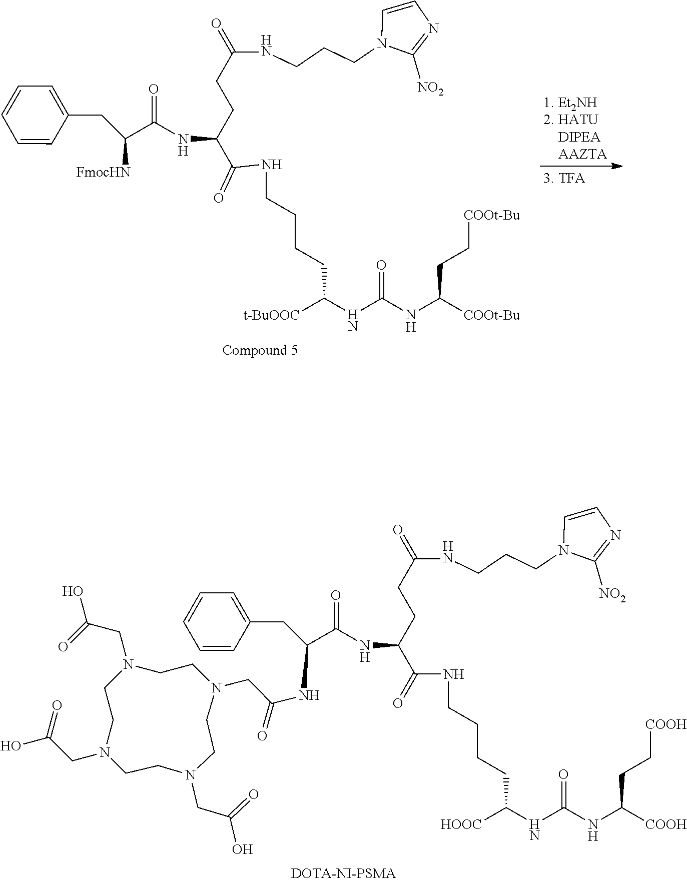

### Synthesis of DOTA-NI-PSMA

Compound 5 (191 mg, 0.17 mmol) was dissolved in dichloromethane (3 mL), followed by the addition of diethylamine (1 mL). The mixture was stirred at room temperature for 3 hours. The solvent was removed by evaporation under reduced pressure to yield a yellow oily intermediate. 1,4,7,10-Tetraazacyclododecane-1,4,7,10-tetraacetic acid tri-tert-butyl ester (DOTA) (34.34 mg, 0.06 mmol) was dissolved in anhydrous N,N-dimethylformamide (2 mL). HATU (29.66 mg, 0.08 mmol) and DIPEA (15.51 mg, 0.12 mmol) under an ice bath and stirred for 30 minutes. The yellow oily intermediate (50 mg, 0.05 mmol) in anhydrous N,N-dimethylformamide (2 mL) was added to the above reaction mixture and stirred overnight at room temperature. The reaction mixture was washed with ethyl acetate and saturated brine (5×). The organic phase was collected and dried over anhydrous sodium sulfate and filtered, and the anhydrous sodium sulfate was removed. After evaporation under reduced pressure to remove the solvent, the residue was purified by flash chromatography (dichloromethane/methanol/aq. ammonia, v/v/v=10/1/0.1) to yield a yellow oily product. The yellow oily product was then deprotected with trifluoroacetic acid to yield crude DOTA-NI-PSMA, which was purified by HPLC (Semi-preparative chromatography column: Eclipse XDB-C18, 5 μm, 9.4×250 mm. Separation conditions: Phase A: 0.1% TFA in H O, Phase B: 0.1% TFA in CH CN. gradient: 0-15 min, 5-100% B; 15-17 min, 100% B; 17-17.5 min, 100-5% B; 17.5-20 min, 5% B. flow rate: 4 mL/min. UV: 280 nm). The target fraction was obtained as a white solid compound DOTA-NI-PSMA through freeze-drying.

### Structural Confirmation of DOTA-NI-PSMA

HRMS: m/z calcd for CH₇₃NO [M+H]⁺: 1134.5061, found: 1134.5046.

### Step 2: ⁶⁸Ga Labeling of the PSMA-Targeted Radioligand DOTA-NI-PSMA Containing a Nitroaromatic Heterocyclic Group

The ⁶⁸Ga labeling route is as follows.

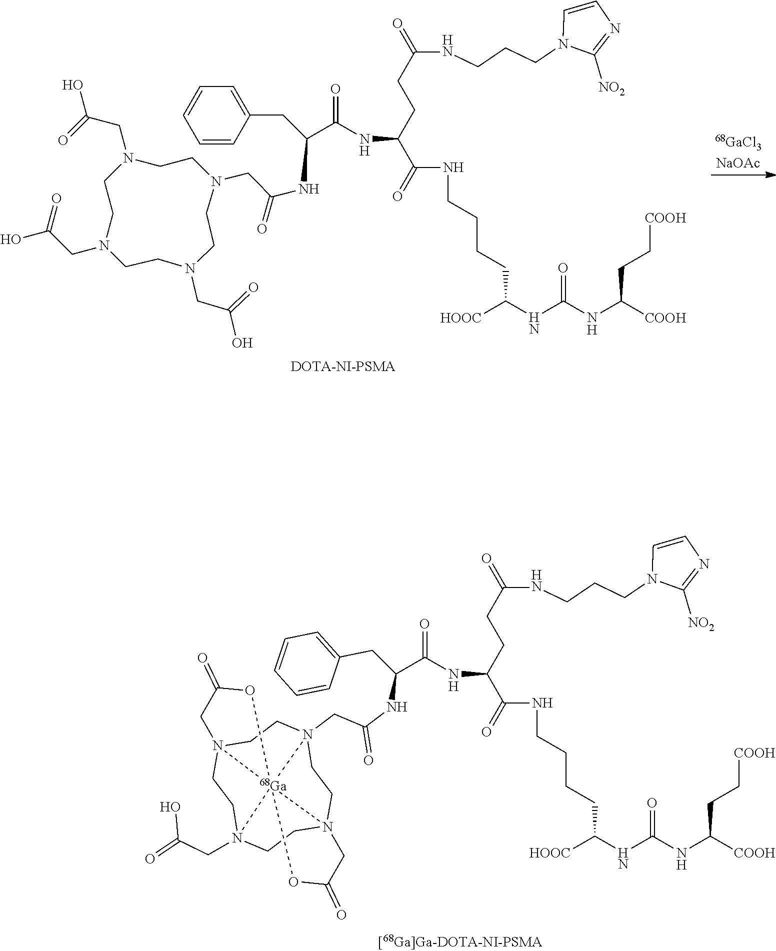

DOTA-NI-PSMA obtained in Step 1 was dissolved in dimethyl sulfoxide (DMSO) to prepare a precursor solution (1 μg/μL). 17 μL of the precursor solution was added to a 10 mL vial, and 72 μL of 3 M sodium acetate solution was added. The germanium-gallium generator (iThemba) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 95° C. for 15 minutes, followed by cooling to room temperature. The radiochemical purity was determined by radio-HPLC equipped with a radioactive detector, confirming a radiochemical yield of >98% for [⁶⁸Ga]Ga-DOTA-NI-PSMA.

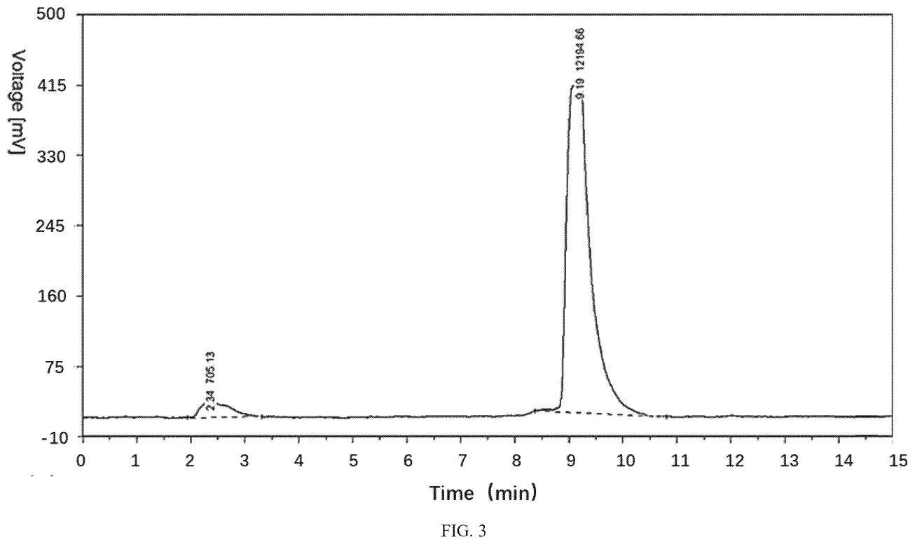

As shown in FIG. 3, FIG. 3 shows the radio-HPLC chromatogram of the labeling reaction mixture for [⁶⁸Ga]Ga-DOTA-NI-PSMA prepared in Embodiment 3 according to the present disclosure, which demonstrates a radiochemical purity exceeding 98%.

### Embodiment 4: Preparation of [⁶⁸Ga]Ga-HBED-CC-NI-PSMA-11

### Step 1: Synthesis of the PSMA-Targeted Radioligand HBED-CC-NI-PSMA-11 Containing a Nitroaromatic Heterocyclic Group

The synthesis route is as follows.

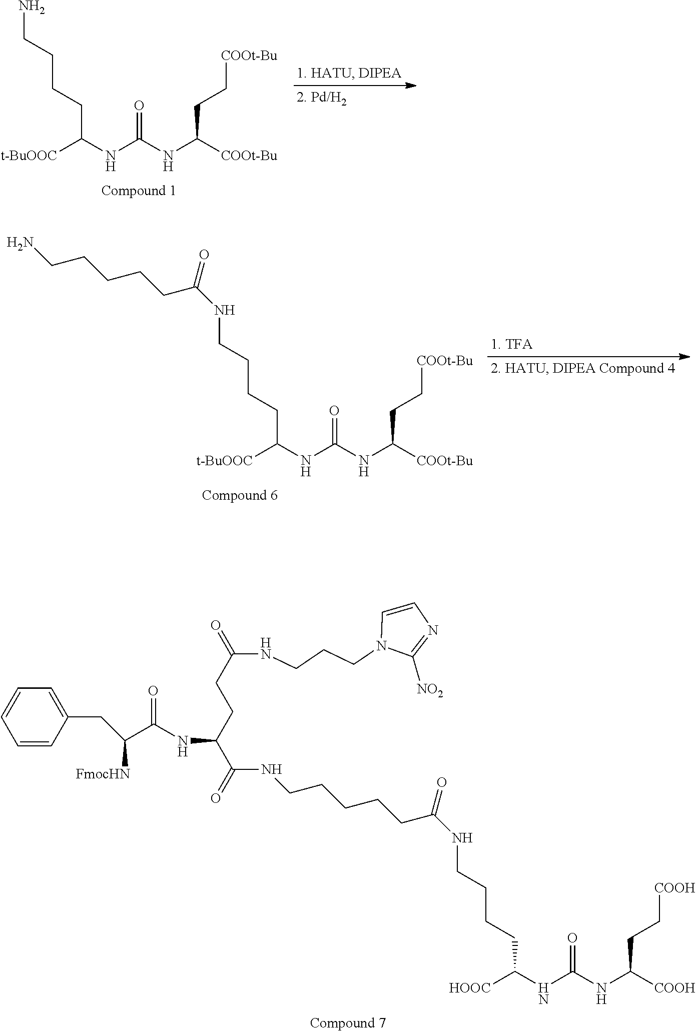

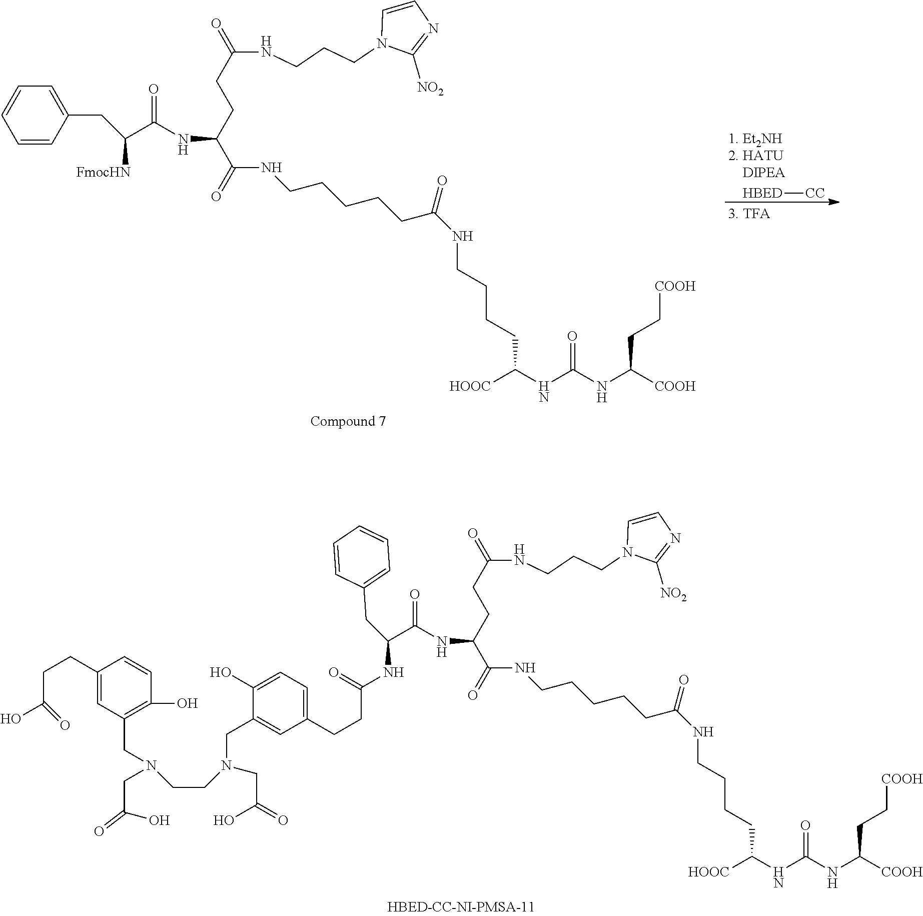

Specifically, the following steps are included.

### (1) Synthesis of Compound 6

CBz-6-aminohexanoic acid (356 mg, 1.34 mmol) was dissolved in anhydrous N,N-dimethylformamide (7 mL). HATU (245 mg, 0.64 mmol) and DIPEA (150 mg, 1.16 mmol) were added under an ice bath and stirred for 30 minutes. Compound 1 (259 mg, 0.53 mmol) in anhydrous N,N-dimethylformamide (5 mL) was added to the reaction mixture and stirred overnight at room temperature. The reaction mixture was washed with saturated brine and ethyl acetate. The organic phase was collected and dried over anhydrous sodium sulfate. After evaporation under reduced pressure to remove the solvent, the residue was purified by silica gel column chromatography (petroleum ether/ethyl acetate, v/v=1/3) to yield a colorless oily product, which was reduced under hydrogen atmosphere to yield Compound 6.

### Structural Confirmation of Compound 6:

HRMS: m/z calcd for CH₅₇NO₈ [M+H]⁺: 601.417, found: 601.4177.

¹H NMR (400 MHz, (CD) SO) δ: 7.83-7.64 (n, 1H), 6.30 (dd, J=16.2, 8.3 Hz, 2H), 4.02 (dd, J=8.5, 5.3 Hz, 1H), 3.94 (dd, J=13.5, 7.7 Hz, 1H), 2.99 (dd, J=12.3, 5.5 Hz, 4H), 2.52-2.46 (m, 2H), 2.34-2.09 (m, 3H), 2.01 (t, J=7.5 Hz, 2H), 1.86 (ddd, J=20.7, 10.4, 6.2 Hz, 1H), 1.72-1.44 (m, 6H), 1.41-1.36 (m, 27H), 1.31-1.13 (m, 6H).

### (2) Synthesis of Compound 7

Compound 4 (500 mg, 0.69 mmol) was dissolved in dichloromethane (2 mL), and trifluoroacetic acid (2 mL) was added. After stirring at room temperature for 30 minutes, the solvent was removed by evaporation under reduced pressure to yield an orange-yellow oily intermediate. The intermediate (287 mg, 0.43 mmol) was dissolved in anhydrous N,N-dimethylformamide (3 mL). HATU (183 mg, 0.48 mmol) and DIPEA (105 mg, 0.81 mmol) were added under an ice bath and stirred for 30 minutes. Compound 6 (240 mg, 0.40 mmol) in anhydrous N,N-dimethylformamide (3 mL) was added to the above reaction mixture and stirred overnight at room temperature. The reaction mixture was washed with ethyl acetate and saturated brine (5×). The organic phase was collected and dried over anhydrous sodium sulfate and filtered, and the anhydrous sodium sulfate was removed. After evaporation under reduced pressure to remove the solvent, the residue was purified by flash chromatography (dichloromethane/methanol/aq. ammonia, v/v/v=10/1/0.1) to yield a pale-yellow solid Compound 7 (305 mg, 0.24 mmol, yield: 61%).

### Structural Confirmation of Compound 7

HRMS: m/z calcd for C₆₅H₉₁NO [M+H]⁺: 1251.6659, found: 1251.6666.

¹H NMR (600 MHz, CDCl) δ: 7.85 (s, 1H), 7.74 (d, J=7.6 Hz, 2H), 7.51 (d, J=7.5 Hz, 2H), 7.37 (ddd, J=7.6, 5.5, 2.0 Hz, 2H), 7.31 (s, 1H), 7.29-7.26 (m, 3H), 7.26-7.24 (m, 1H), 7.24-7.17 (m, 2H), 7.08 (s, 1H), 6.96 (s, 1H), 5.86 (d, J=5.2 Hz, 1H), 4.54-4.44 (m, 2H), 4.44-4.38 (m, 3H), 4.37-4.28 (m, 2H), 4.23-4.10 (m, 2H), 3.37-3.23 (m, 3H), 3.22-3.11 (m, 4H), 3.08-3.00 (m, 1H), 2.36-2.26 (m, 4H), 2.22 (t, J=6.3 Hz, 2H), 2.13-1.91 (m, 6H), 1.88-1.74 (m, 2H), 1.70-1.63 (m, 1H), 1.62-1.56 (m, 2H), 1.55-1.48 (m, 3H), 1.47-1.42 (m, 20H), 1.40 (s, 10H), 1.35-1.24 (m, 6H).

### (3) Synthesis of HBED-CC-NI-PSMA-11

Compound 7 (305 mg, 0.24 mmol) was dissolved in dichloromethane (5 mL), followed by the addition of diethylamine (1 mL). After stirring at room temperature for 3 hours, the solvent was removed by evaporation under reduced pressure to yield a yellow oily intermediate. HBED-CC (33 mg, 0.05 mmol) was dissolved in anhydrous N,N-dimethylformamide (2 mL). HATU (25 mg, 0.07 mmol) and DIPEA (13 mg, 0.10 mmol) were added under an ice bath and stirred for 30 minutes. The yellow oily intermediate (50 mg, 0.05 mmol) in anhydrous N,N-dimethylformamide (2 mL) was added to the above reaction mixture and stirred overnight at room temperature. The reaction mixture was washed with ethyl acetate and saturated brine (5×). The organic phase was collected and dried over anhydrous sodium sulfate and filtered, and the anhydrous sodium sulfate was removed. After evaporation under reduced pressure to remove the solvent, the residue was purified by flash chromatography (dichloromethane/methanol/aq. ammonia, v/v/v=10/1/0.1) to yield a yellow oily product. The yellow oily product was then deprotected with trifluoroacetic acid to yield crude HBED-CC-NI-PSMA-11, which was purified by HPLC (Semi-preparative chromatography column: Eclipse XDB-C18, 5 μm, 9.4×250 mm. Separation conditions: Phase A: 0.1% TFA in H O, Phase B: 0.1% TFA in CH CN. gradient: 0-15 min, 5-65% B; 15-16 min, 65-100% B; 16-19 min, 100% B; 19-20 min, 100-5% B; 20-23 min, 5% B. flow rate: 4 mL/min. UV: 280 nm). The target fraction was obtained as a white solid compound HBED-CC-NI-PSMA-11 through freeze-drying.

### Structural Confirmation of HBED-CC-NI-PsMA-11

HRMS: m/z calcd for C₆₄H₈₇NO [M+H]⁺: 1375.6052, found: 1375.6068.

### Step 2: ⁶⁸Ga Labeling of the PSMA-Targeted Radioligand HBED-CC-NI-PSMA-11 Containing a Nitroaromatic Heterocyclic Group

The ⁶⁸Ga labeling route is as follows.

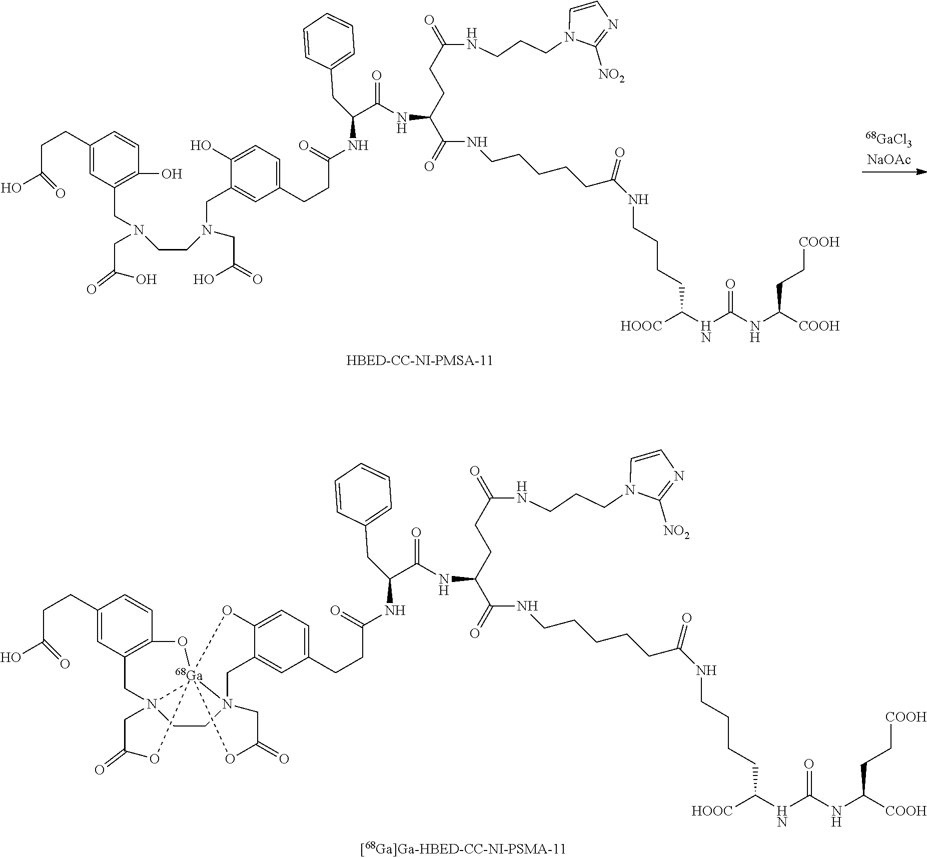

HBED-CC-NI-PSMA-11 obtained in Step 1 was dissolved in dimethyl sulfoxide (DMSO) to prepare a precursor solution (1 μg/L). 5 μL of the precursor solution was added to a 10 mL vial, and 135 μL of 3 M sodium acetate solution was added. The germanium-gallium generator (iThemba) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 50° C. for 10 minutes, followed by cooling to room temperature. The radiochemical purity was determined by radio-HPLC equipped with a radioactive detector, confirming a radiochemical yield of >98% for [⁶⁸Ga]Ga-HBED-CC-NI-PSMA-11.

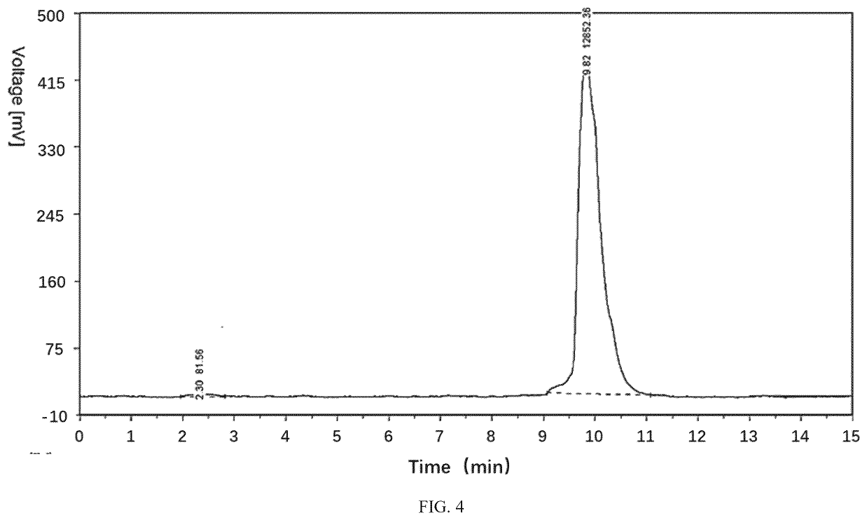

As shown in FIG. 4, FIG. 4 shows the radio-HPLC chromatogram of the labeling reaction mixture for [⁶⁸Ga]Ga-HBED-CC-NI-PSMA-11 prepared in Embodiment 4 according to the present disclosure, which demonstrates a radiochemical purity exceeding 98%.

### Embodiment 5: Preparation of [⁶⁸Ga]Ga-AAZTA-NI-PSMA-11

### Step 1: Synthesis of the PSMA-Targeted Radioligand AAZTA-NI-PSMA-11 Containing a Nitroaromatic Heterocyclic Group

The synthesis route is as follows.

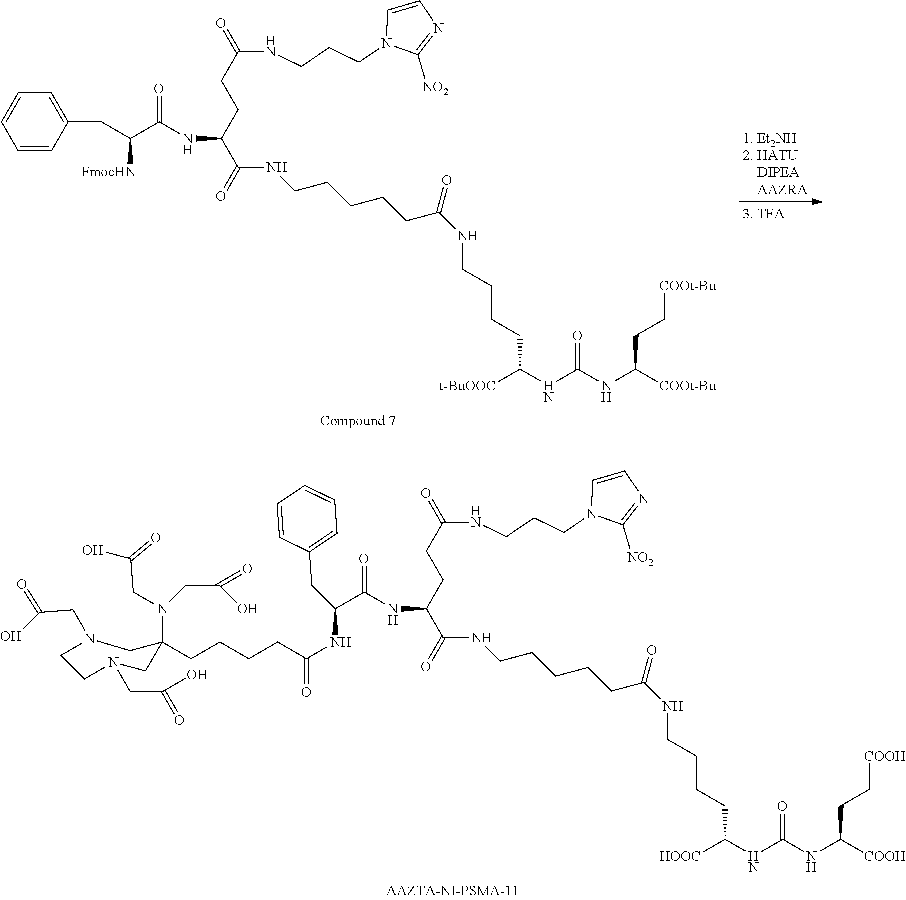

Compound 7 (305 mg, 0.24 mmol) was dissolved in dichloromethane (5 mL), followed by the addition of diethylamine (1 mL). After stirring at room temperature for 3 hours, the solvent was removed by evaporation under reduced pressure to yield a yellow oily intermediate. AAZTA (33.57 mg, 0.05 mmol) was dissolved in anhydrous N,N-dimethylformamide (2 mL). HATU (25 mg, 0.07 mmol) and DIPEA (13 mg, 0.10 mmol) were added under an ice bath and stirred for 30 minutes. The yellow oily intermediate (50 mg, 0.05 mmol) in anhydrous N,N dimethylformamide (2 mL) was added to the above reaction mixture and stirred overnight at room temperature. The reaction mixture was washed with ethyl acetate and saturated brine (5×). The organic phase was collected and dried over anhydrous sodium sulfate and filtered, and the anhydrous sodium sulfate was removed. After evaporation under reduced pressure to remove the solvent, the residue was purified by flash chromatography (dichloromethane/methanol/aq. ammonia, v/v/v=10/1/0.1) to yield a yellow oily product. The yellow oily product was then deprotected with trifluoroacetic acid to yield crude AAZTA-NI-PSMA-11, which was purified by HPLC (Semi-preparative chromatography column: Eclipse XDB-C18, 5 μm, 9.4×250 mm. Separation conditions: Phase A: 0.1% TFA in H O, Phase B: 0.1% TFA in CH CN. gradient: 0-15 min, 5-100% B; 15-17 min, 100% B; 17-17.5 min, 100-5% B; 17.5-20 min, 5% B. flow rate: 4 mL/min. UV: 280 nm). The target fraction was obtained as a white solid compound AAZTA-NI-PSMA-11 through freeze-drying.

### Structural Confirmation of AAZTA-NI-PSMA-11

HRMS: m/z calcd for C₅₆H₈₄NO [M+H]⁺: 1290.5848, found: 1290.5859.

### Step 2: ⁶⁸Ga Labeling of the PSMA-Targeted Radioligand AAZTA-NI-PSMA-11 Containing a Nitroaromatic Heterocyclic Group

The ⁶⁸Ga labeling route is as follows.

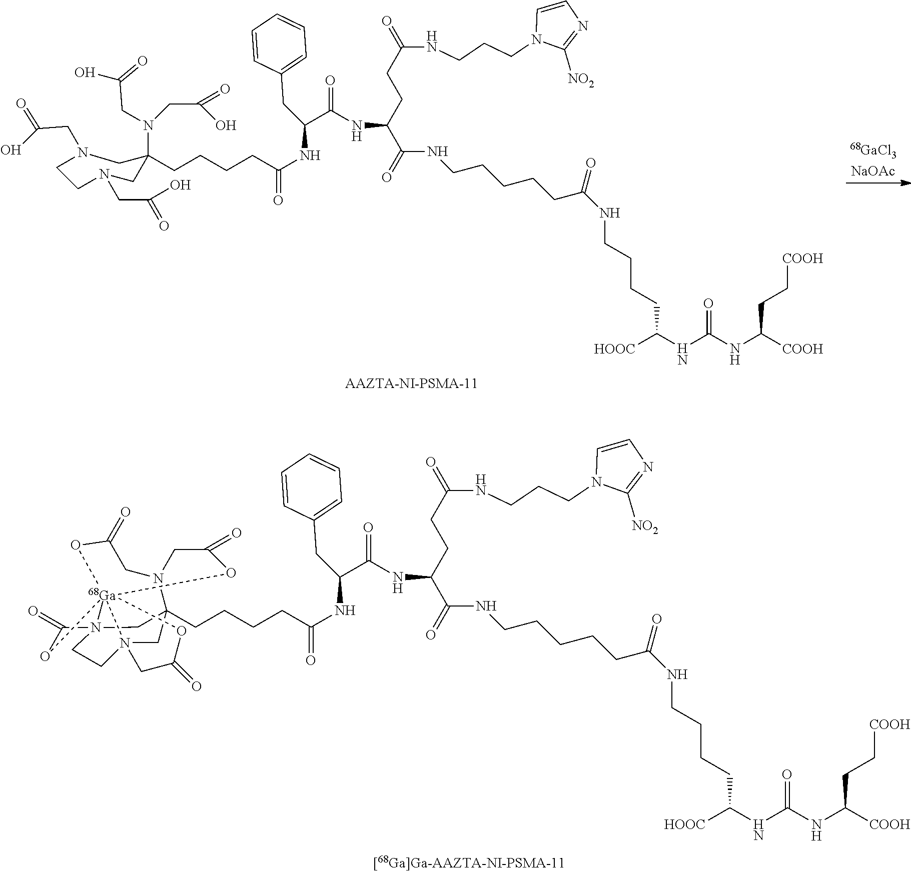

AZTA-NI-PSMA-11 obtained in Step 1 was dissolved in dimethyl sulfoxide (DMSO) to prepare a precursor solution (1 μg/μL). 10 μL of the precursor solution was added to a 10 mL vial, and 135 μL of 3 M sodium acetate solution was added. The germanium-gallium generator (iThemba) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 50° C. for 10 minutes, followed by cooling to room temperature. The radiochemical purity was determined by radio-HPLC equipped with a radioactive detector, confirming a radiochemical yield of >98% for [⁶⁸Ga]Ga-AAZTA-NI-PSMA-11.

As shown in FIG. 5, FIG. 5 shows the radio-HPLC chromatogram of the labeling reaction mixture for [⁶⁸Ga]Ga-AAZTA-NI-PSMA-11 prepared in Embodiment 5 according to the present disclosure, which demonstrates a radiochemical purity exceeding 98%.

### Embodiment 6: Preparation of [⁶⁸Ga]Ga-DOTA-NI-PSMA-11

### Step 1: Synthesis of the PSMA-Targeted Radioligand DOTA-NI-PSMA-11 Containing a Nitroaromatic Heterocyclic Group

The synthesis route is as follows.

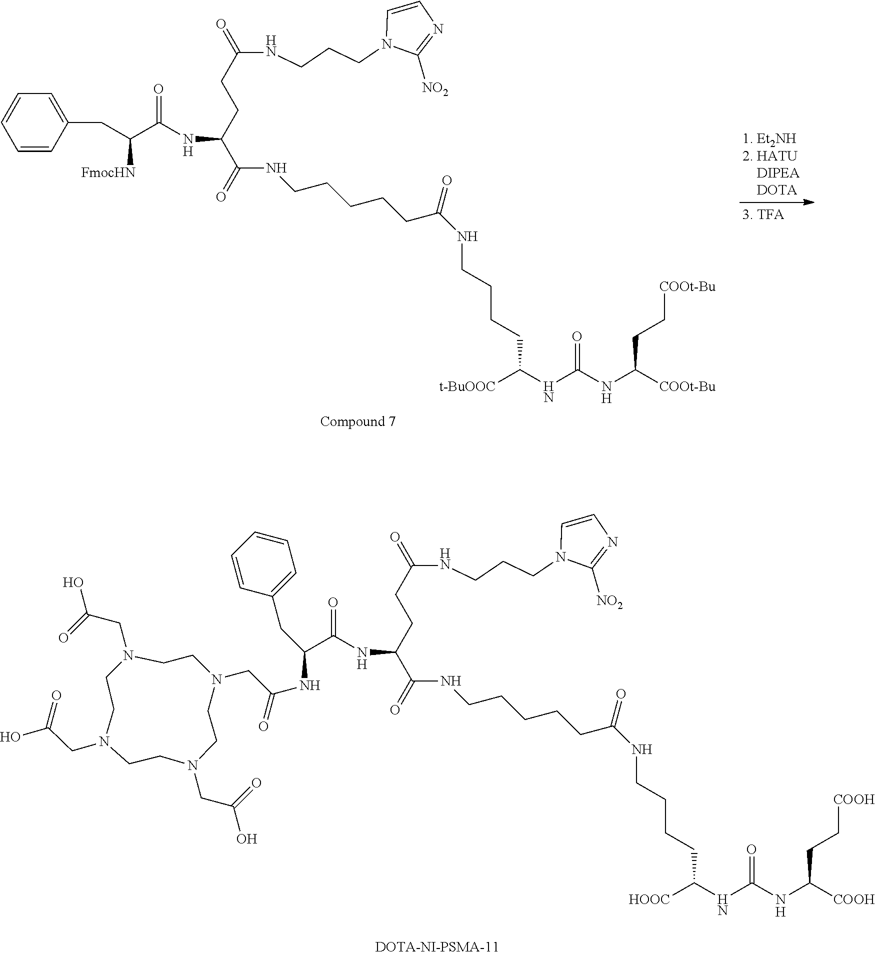

Compound 7 (305 mg, 0.24 mmol) was dissolved in dichloromethane (5 mL), followed by the addition of diethylamine (1 mL). After stirring at room temperature for 3 hours, the solvent was removed by evaporation under reduced pressure to yield a yellow oily intermediate. DOTA (29 mg, 0.05 mmol) was dissolved in anhydrous N,N-dimethylformamide (2 mL). HATU (25 mg, 0.07 mmol) and DIPEA (13 mg, 0.10 mmol) were added under an ice bath and stirred for 30 minutes. The yellow oily intermediate (50 mg, 0.05 mmol) in anhydrous N,N dimethylformamide (2 mL) was added to the above reaction mixture and stirred overnight at room temperature. The reaction mixture was washed with ethyl acetate and saturated brine (5×). The organic phase collected and dried over anhydrous sodium sulfate and filtered, and the anhydrous sodium sulfate was removed. After evaporation under reduced pressure to remove the solvent, the residue was purified by flash chromatography (dichloromethane/methanol/aq. ammonia, v/v/v=10/1/0.1) yielded a yellow oily product. The yellow oily product was then deprotected with trifluoroacetic acid to yield crude DOTA-NI-PSMA-11, which was purified by HPLC (Semi-preparative chromatography column: Eclipse XDB-C18, 5 μm, 9.4×250 mm. Separation conditions: Phase A: 0.1% TFA in H O, Phase B: 0.1% TFA in CH CN. gradient: 0-15 min, 5-100% B; 15-17 min, 100% B; 17-17.5 min, 100-5% B; 17.5-20 min, 5% B. flow rate: 4 mL/min. UV: 280 nm). The target fraction was obtained as a white solid compound DOTA-NI-PSMA-11 through freeze-drying.

### Structural Confirmation of DOTA-NI-PSMA-11

HRMS: m/z calcd for C₅₄H₈₃NO [M+H]⁺: 1247.5902, found: 1247.5896.

### Step 2: ⁶⁸Ga Labeling of the PSMA-Targeted Radioligand DOTA-NI-PSMA-11 Containing a Nitroaromatic Heterocyclic Group

The ⁶⁸Ga labeling route is as follows.

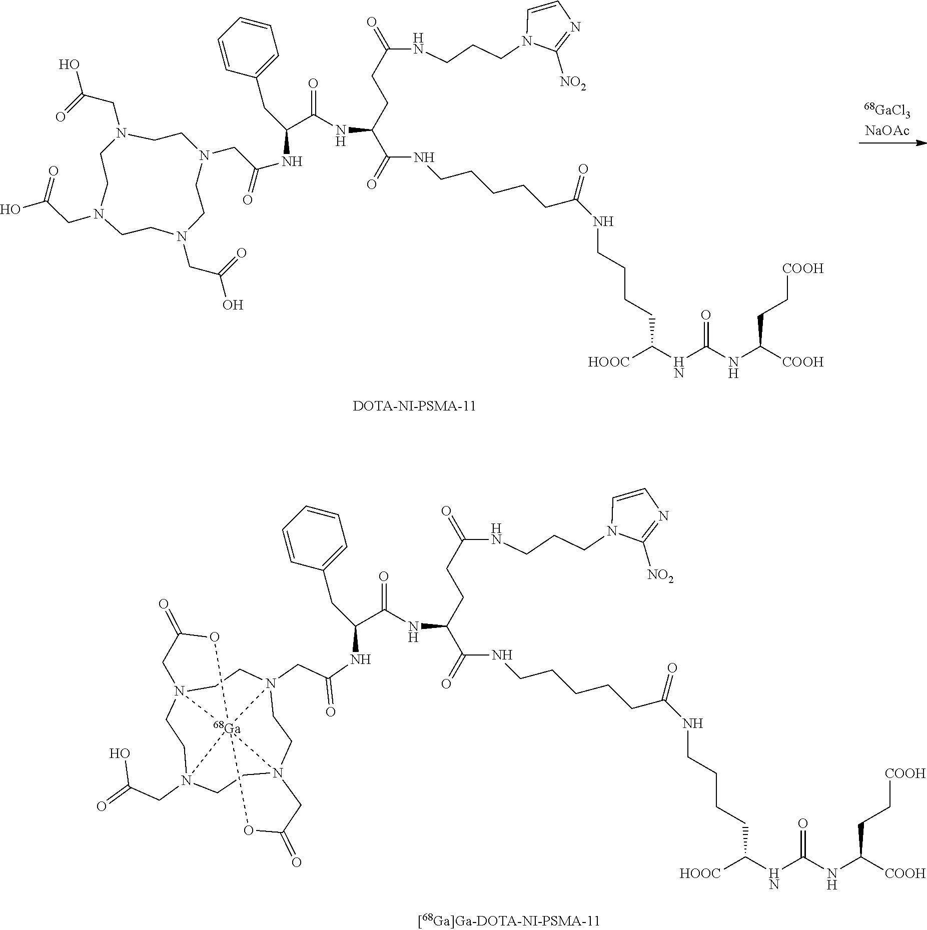

DOTA-NI-PSMA-11 obtained in Step 1 was dissolved in dimethyl sulfoxide (DMSO) to prepare a precursor solution (1 μg/μL). 25 μL of the precursor solution was added to a 10 mL vial, and 72 μL of 3 M sodium acetate solution was added. The germanium-gallium generator (iThemba) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 95° C. for 15 minutes, followed by cooling to room temperature. The radiochemical purity was determined by radio-HPLC equipped with a radioactive detector, confirming a radiochemical yield of >98% for [⁶⁸Ga]Ga-DOTA-NI-PSMA-11.

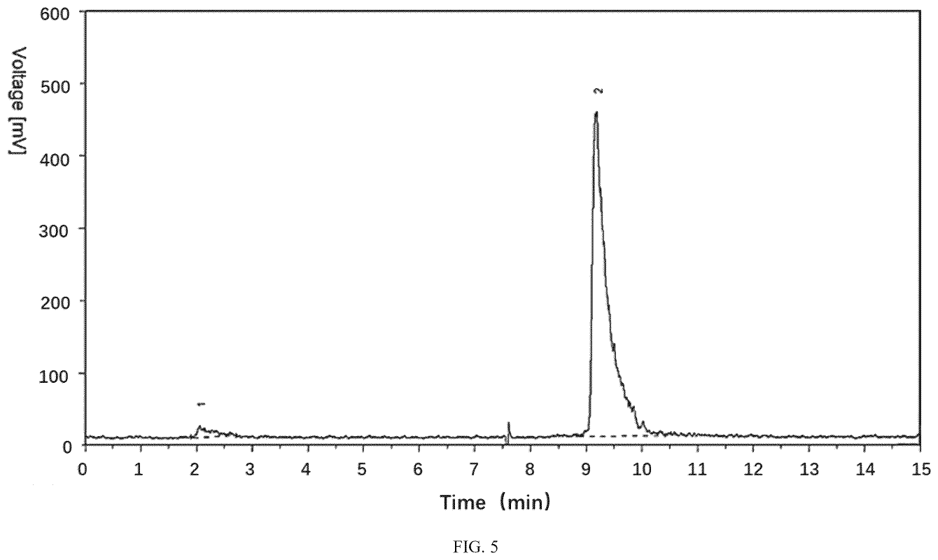

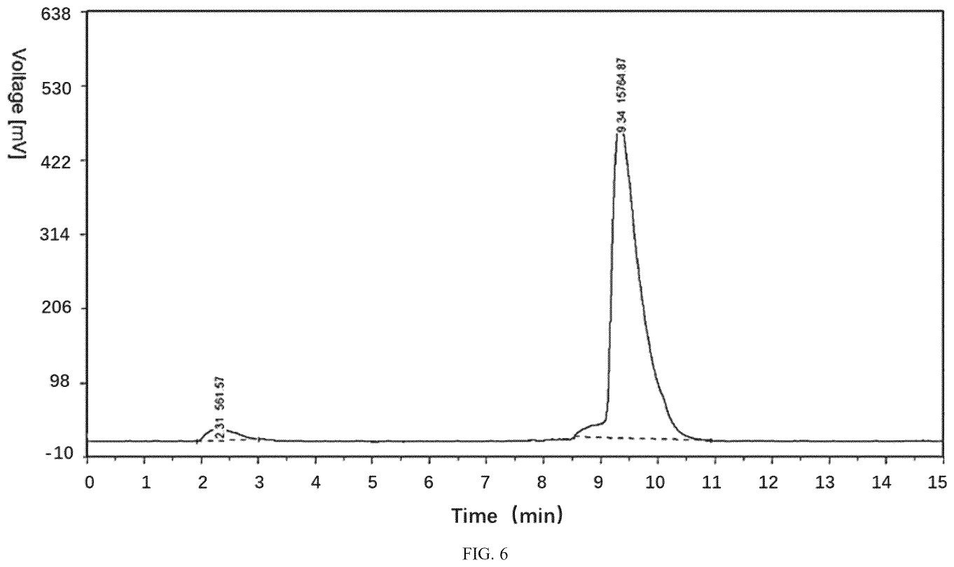

As shown in FIG. 6, FIG. 6 shows the radio-HPLC chromatogram of the labeling reaction mixture for [⁶⁸Ga]Ga-DOTA-NI-PSMA-11 prepared in Embodiment 6 according to the present disclosure, which demonstrates a radiochemical purity exceeding 98%.

### Embodiment 7: Preparation of [⁶⁸Ga]Ga-AAZTA-NI-PSMA-093

### Step 1: Synthesis of the PSMA-Targeted Radioligand AAZTA-NI-PSMA-093 Containing a Nitroaromatic Heterocyclic Group

The synthesis route is as follows.

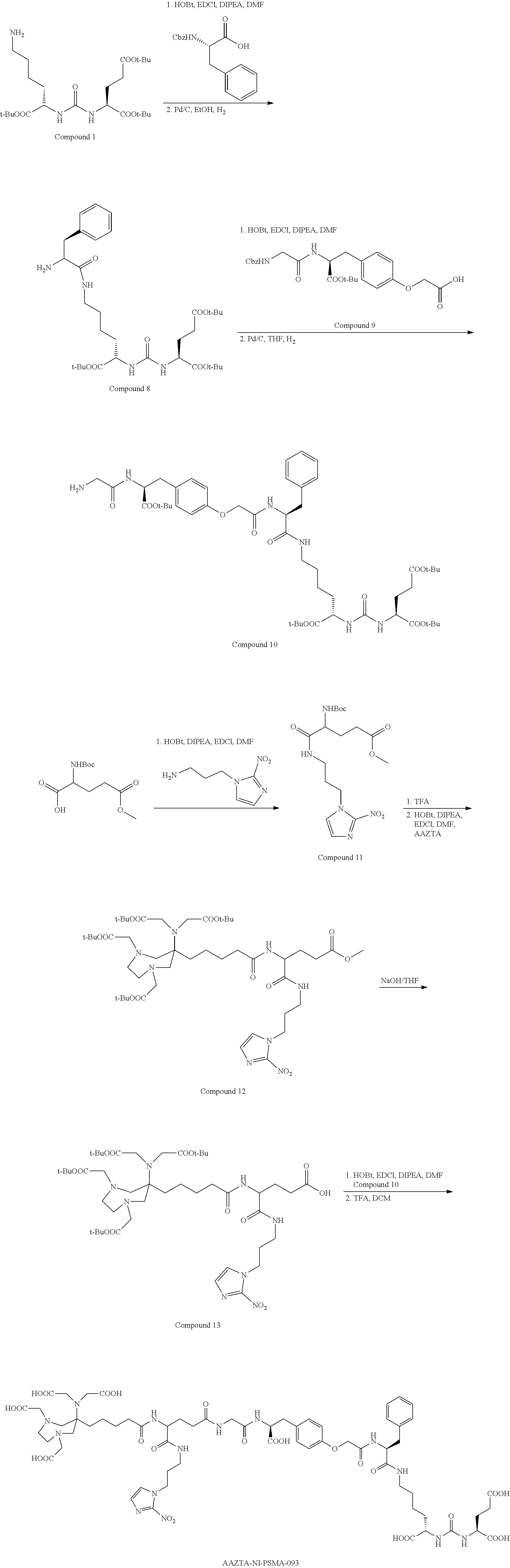

Specifically, the following steps are included.

### (1) Synthesis of Compound 8

N-Benzyloxycarbonyl-L-phenylalanine (N-Cbz-L-Phe, 1.42 g, 4.73 mmol) was dissolved in N,N-dimethylformamide (11 mL). At 0° C., hydroxybenzotriazole (HOBt, 872 mg, 6.45 mmol), N,N-diisopropylethylamine (DIPEA, 2.20 mL, 1.72 g, 12.9 mmol), and 1-ethyl-(3-dimethylaminopropyl)carbodiimide hydrochloride (EDCl, 1.24 g, 6.45 mmol) were added. Compound 1 (2.10 g, 4.30 mmol) in N,N dimethylformamide (15 mL) was added dropwise to the above mixture, which was stirred at room temperature for 27 hours. The mixture was extracted with saturated sodium chloride solution and ethyl acetate. The organic phase was washed with saturated sodium chloride solution (3×), dried over anhydrous sodium sulfate, filtered, and concentrated. Purification by silica gel column chromatography (dichloromethane/methanol/aq. ammonia, v/v/v=95/5/0.5) yielded a white solid (1.29 g, 1.67 mmol, yield: 38.9%). This solid (1.29 g, 1.67 mmol) was dissolved in anhydrous ethanol (20 mL), and 10% Pd/C (88.1 mg) was added. The mixture was stirred under a hydrogen atmosphere at room temperature for 27 hours and then filtered through Celite, and the filtrate was concentrated by evaporation under reduced pressure to afford Compound 8 as a brown oily product (981 mg, 1.55 mmol, yield: 92.5%).

### Structural Confirmation of Compound 8

HRMS: m/z calcd for CH₅₅NO₈ [M+H]⁺: 635.4014, found: 635.4011.

¹H NMR (600 MHz, CDCl) δ: 7.33-7.18 (m, 5H), 5.40 (dd, J=10.6, 8.1 Hz, 2H), 4.30 (dtd, J=28.9, 8.0, 4.9 Hz, 2H), 3.59 (dd, J=9.3, 4.2 Hz, 1H), 3.30-3.16 (m, 3H), 2.67 (dd, J=13.7, 9.3 Hz, 1H), 2.37-2.23 (m, 2H), 2.09-2.01 (m, 1H), 1.88-1.70 (m, 3H), 1.65-1.56 (m, 1H), 1.51-1.46 (n, 2H), 1.45-1.41 (m, 27H), 1.37-1.28 (m, 2H).

### (2) Synthesis of Compound 10

Compound 8 (959 mg, 1.51 mmol) was dissolved in N, A dimethylformamide (15 mL). At 0° C., HOBt (237 mg, 1.75 mmol), DIPEA (0.60 mL, 469 mg, 3.63 mmol), and EDCl (337 mg, 1.75 mmol) were added. Compound 9 (569 mg, 1.17 mmol) in N,N-dimethylformamide (15 mL) was added dropwise. The reaction mixture was stirred at room temperature for 13.5 hours and then extracted with saturated sodium chloride solution and ethyl acetate. The organic phase was washed with saturated sodium chloride solution (3×), dried over anhydrous sodium sulfate, filtered, and concentrated. Purification by silica gel column chromatography (dichloromethane/methanol/aq. ammonia, v/v/v=95/5/0.5) yielded Compound 10 as a white solid product (725 mg, 0.657 mmol, yield: 56.2%).

### Structural Confirmation of Compound 10:

HRMS: m/z calcd for C₅₈H₈₃N₆O [M+H]⁺: 1103.5910, found: 1103.5927.

¹H NMR (600 MHz, CDCl) δ: 8.09 (brs, 1H), 7.56 (brs, 1H), 7.39-7.28 (m, 5H), 7.28-7.17 (m, 5H), 6.87 (d, J=7.7 Hz, 2H), 6.59 (d, J=7.8 Hz, 2H), 5.80 (brs, 1H), 5.06 (d, J=12.1 Hz, 2H), 4.92 (d, J=12.0 Hz, 1H), 4.81 (brs, 1H), 4.76-4.59 (m, 1H), 4.52 (brs, 1H), 4.41 (brs, 2H), 4.05 (d, J=14.3 Hz, 1H), 3.86 (dd, J=17.4, 3.3 Hz, 1H), 3.44 (brs, 2H), 3.27-3.17 (m, 2H), 3.15-2.96 (m, 2H), 2.88 (brs, 1H), 2.44-2.28 (m, 2H), 2.12-2.02 (m, 1H), 1.87-1.79 (m, 1H), 1.58 (d, J=6.3 Hz, 2H), 1.53-1.28 (m, 36H), 1.28-1.19 (m, 2H), 1.05 (brs, 1H).

### (6) Synthesis of Compound 11

Compound 2 (432 mg, 1.60 mmol) was dissolved in trifluoroacetic acid (TFA, 4 mL) and stirred at room temperature for 30 minutes. The solvent was removed by evaporation under reduced pressure to yield a white solid intermediate. This intermediate (812 mg, 3.00 mmol) was dissolved in dichloromethane (6 mL), and TFA (2.5 mL) was added. The mixture was stirred at room temperature for 40 minutes and subjected to evaporation under reduced pressure to remove the solvent to afford a yellow oily product. N-tert-Butoxycarbonyl-L-glutamic acid 5-methyl ester (530 mg, 2.03 mmol) was dissolved in N,N-dimethylformamide (10 mL). At 0° C., HOBt (410 mg, 3.03 mmol), DIPEA (0.90 mL, 704 mg, 5.45 mmol), and EDCl (572 mg, 2.99 mmol) were added. The yellow oily product in N,N-dimethylformamide (10 mL) was added dropwise. The reaction mixture was stirred at room temperature for 26 hours and then extracted with saturated sodium chloride solution and ethyl acetate. The organic phase was washed with saturated sodium chloride solution (3×), dried over anhydrous sodium sulfate, filtered, and concentrated. Purification by silica gel column chromatography (dichloromethane/methanol/aq. ammonia, v/v/v=90/9/0.9) yielded Compound 11 as a yellow oily product (434 mg, 1.05 mmol, yield: 35.0%).

### Structural Confirmation of Compound 11

HRMS: m/z calcd for CHN₅O₇ [M+H]⁺: 414.1983, found: 414.1978.

¹H NMR (400 MHz, CDCl) δ: 7.32 (s, 1H), 7.14 (s, 1H), 6.71 (br s, 1H), 5.36 (d, J=7.1 Hz, 1H), 4.44 (t, J=6.9 Hz, 2H), 4.11 (q, J=7.1 Hz, 1H), 3.69 (s, 3H), 3.34 (dd, J=12.5, 6.2 Hz, 2H), 2.58-2.36 (m, 2H), 2.20-2.02 (m, 3H), 2.01-1.86 (m, 1H), 1.43 (s, 9H).

### (7) Synthesis of Compound 12

Compound 11 (152 mg, 0.367 mmol) was dissolved in dichloromethane (6 mL), and TFA (2 mL) was added. The mixture was stirred at room temperature for 1 hour and subjected to evaporation under reduced pressure to remove the solvent to yield a yellow oily product. AAZTA (149 mg, 0.223 mmol) was dissolved in N,N-dimethylformamide (7 mL). At 0° C., O-(7-Azabenzotriazol-1-yl)-N,N,N′,N′-tetramethyluronium hexafluorophosphate (HATU, 161 mg, 0.423 mmol) and DIPEA (0.05 mL, 37.1 mg, 0.287 mmol) were added and stirred for 30 minutes. The yellow oily product in N,N-dimethylformamide (5 mL) was added dropwise. The reaction mixture was stirred at room temperature for 27 hours. The mixture was extracted with saturated sodium chloride solution and ethyl acetate. The organic phase was washed with saturated sodium chloride solution (3×), dried over anhydrous sodium sulfate, filtered, and concentrated. Purification by silica gel column chromatography (dichloromethane/methanol/aq. ammonia, v/v/v=100/5/0.5) yielded Compound 12 as a yellow oily product (71.9 mg, 0.0743 mmol, yield: 33.8%).

Structural confirmation of Compound 12

HRMS: m/z calcd for CH₇₉N₈O [M+H]⁺: 967.5710, found: 967.5715.

¹H NMR (600 MHz, CDCl) δ: 7.43 (s, 1H), 7.11 (s, 1H), 6.88-6.78 (m, 1H), 6.74-6.66 (m, 1H), 4.50-4.30 (m, 3H), 3.68 (s, 3H), 3.66-3.55 (m, 2H), 3.54 (s, 2H), 3.47-3.36 (m, 3H), 3.34-3.25 (m, 3H), 3.08-2.98 (m, 2H), 2.90-2.81 (m, 1H), 2.73-2.66 (m, 1H), 2.52-2.39 (m, 2H), 2.37-2.25 (m, 2H), 2.11-1.99 (m, 4H), 1.72 (s, 8H), 1.50-1.41 (n, 36H).

### (8) Synthesis of Compound 13

Compound 12 (56.4 mg, 58.3 μmol) was dissolved in tetrahydrofuran (3 mL). A 1 M sodium hydroxide solution (1.0 mL, 1.0 mmol) was added dropwise and stirred at room temperature for 8 hours. The pH was adjusted to 6 with 1 M hydrochloric acid. The mixture was extracted with saturated sodium chloride solution and ethyl acetate. The aqueous phase was washed with ethyl acetate (3×), and combined with the organic phase. The mixture was dried over anhydrous sodium sulfate, filtered, and concentrated. Purification by silica gel column chromatography (dichloromethane/methanol/aq. ammonia, v/v/v=90/9/0.9) yielded Compound 13 as a white solid product (18.6 mg, 19.5 μmol, yield: 32.5%).

### Structural Confirmation of Compound 13:

HRMS: m/z calcd for CH₇₇N₈O [M+H]⁺: 953.5553, found: 953.5543.

### (9) Synthesis of AAZTA-NI-PSMA-093

Compound 13 (18.6 mg, 19.5 μmol) was dissolved in N,N-dimethylformamide (3 mL). At 0° C., HATU (10.2 mg, 26.8 μmol) and DIPEA (8.30 mg, 64.2 μmol) were added and stirred for 30 minutes. Compound 10 in N,N-dimethylformamide (2 mL) was added dropwise. The reaction mixture was stirred at room temperature for 24 hours and then extracted with saturated sodium chloride solution and ethyl acetate. The organic phase was washed with saturated sodium chloride solution (3×), dried over anhydrous sodium sulfate, filtered, and concentrated. Purification by silica gel column chromatography (dichloromethane/methanol/aq. ammonia, v/v/v=100/5/0.5) yielded a yellow oily product (11.5 mg, 6.04 μmol, yield: 30.2%). The yellow oily product (11.5 mg, 6.04 μmol) was dissolved in dichloromethane (2 mL), and TFA (2 mL) was added. The mixture was stirred at room temperature for 3 hours and subjected to evaporation under reduced pressure to remove the solvent to yield a yellow solid product. The yellow solid product was dissolved in dimethyl sulfoxide (1.0 mL) and purified by semi-preparative HPLC to afford AAZTA-NI-PSMA-093 as a white solid product (2.50 mg, 4.54 μmol, yield: 75.2%).

### Structural Confirmation of AAZTA-NI-PSMA-093

HRMS: m/z calcd for C₆₃H₈₇NO [M+H]⁺: 1511.6536, found: 1511.6548.

### Step 2: ⁶⁸Ga Labeling of the PSMA-Targeted Radioligand AAZTA-NI-PSMA-093 Containing a Nitroaromatic Heterocyclic Group

The ⁶⁸Ga labeling route is as follows.

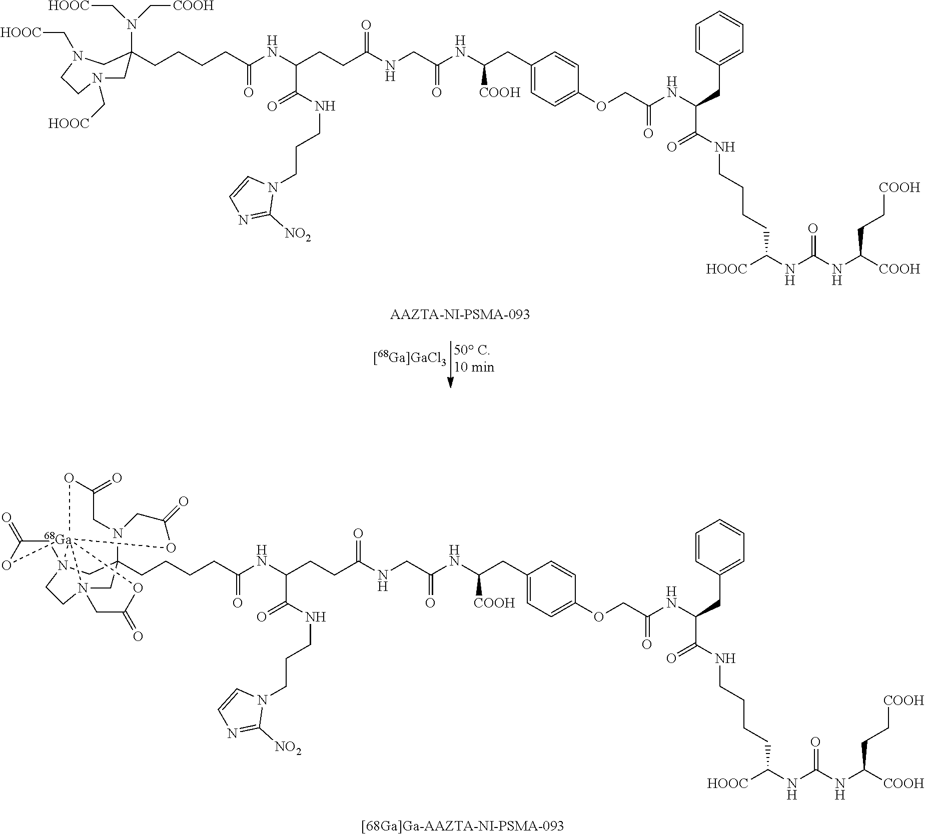

A dimethyl sulfoxide (DMSO) solution containing AAZTA-NI-PSMA-093 (20 nmol) was added to a 3 M sodium acetate buffer (150 μL). The germanium-gallium generator (iThemba Laboratories, 740 MBq, 20 mCi) was eluted with high-purity hydrochloric acid to obtain a [⁶⁸Ga]GaCl₃ hydrochloric acid solution. A 300 μL aliquot of this solution was added to the mixture of the radioligand and sodium acetate. After thorough mixing, the reaction mixture was diluted with water to a total volume of 500 μL and incubated at 50° C. for 10 minutes, followed by cooling to room temperature. The radiochemical purity was determined by radio-HPLC equipped with a radioactive detector, confirming a radiochemical yield of >98% for [⁶⁸Ga]Ga-AAZTA-NI-PSMA-093.

As shown in FIG. 7, FIG. 7 shows the radio-HPLC chromatogram of the labeling reaction mixture for [⁶⁸Ga]Ga-AAZTA-NI-PSMA-093 prepared in Embodiment 7 according to the present disclosure, which demonstrates a radiochemical purity exceeding 98%.

### Embodiment 8: Preparation of [⁶⁸Ga]Ga-NI-HBED-CC-PSMA-11

### Step 1: Synthesis of the PSMA-Targeted Radioligand NI-HBED-CC-PSMA-11 Containing a Nitroaromatic Heterocyclic Group

The synthesis route is as follows.

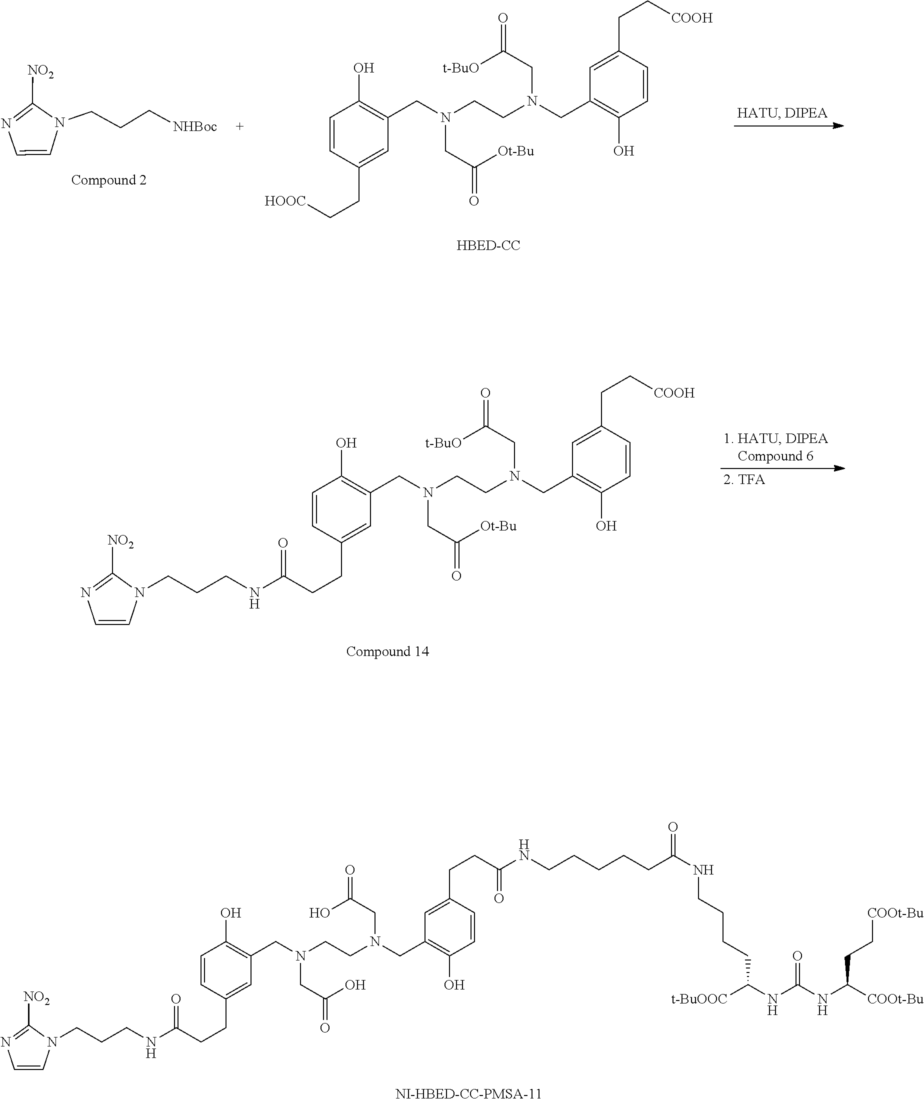

Specifically, the following steps are included.

### (1) Synthesis of Compound 14

HBED-CC (200 mg, 0.310 mmol) was dissolved in anhydrous N,-dimethylformamide (10 mL). HATU (141 mg, 0.372 mmol) and DIPEA (80.0 mg, 0.620 mmol) were sequentially added under an ice bath and stirred. Compound 2 was dissolved in trifluoroacetic acid (2 mL) and stirred for 1 hour. The solvent was removed by evaporation under reduced pressure to yield a white intermediate solid. This intermediate (52.8 mg, 0.310 mmol) in anhydrous N,N dimethylformamide (5 mL) was added dropwise to the above reaction mixture and stirred at room temperature for 4 hours. The mixture was extracted with saturated brine (30 mL) and ethyl acetate. The organic phase was washed with deionized water (2×30 mL) and concentrated by evaporation under reduced pressure. Purification by silica gel column chromatography (dichloromethane/methanol/aq. ammonia, v/v/v=90/10/1) yielded Compound 14 as a colorless product (68.7 mg, 0.0863 mmol, yield: 27.8%).

### Structural Confirmation of Compound 14:

HRMS: m/z calcd for C₅₀H₇₆N₆O [M+H]⁺: 969.5543, found: 969.5542.

¹H NMR (400 MHz, CD OD) δ: 7.71 (d, J=2.1 Hz, 1H), 7.39-7.28 (m, 1H), 7.23-7.09 (m, 3H), 7.06-6.93 (m, 1H), 6.89 (d, J=8.4 Hz, 1H), 6.70 (d, J=8.4 Hz, 1H), 6.47 (s, 1H), 5.77 (d, J=4.5 Hz, 1H), 5.16-4.85 (m, 1H), 4.74 (dd, J=14.1, 5.9 Hz, 1H), 4.56-4.19 (m, 3H), 3.94-3.74 (m, 1H), 3.39 (d, J=16.3 Hz, 2H), 3.26-2.82 (m, 4H), 2.34 (ddd, J=30.4, 18.4, 13.8 Hz, 2H), 2.08 (tdd, J=14.7, 10.1, 4.6 Hz, 1H), 1.84 (ddd, J=19.8, 12.3, 7.9 Hz, 1H), 1.72-1.00 (m, 36H).

### (2) Synthesis of NI-HBED-CC-PSMA-11

Compound 14 (20.1 mg, 25.1 μmol) was dissolved in anhydrous N,N-dimethylformamide (5 mL). Under an ice bath, HATU (14.3 mg, 30.1 μmol) and DIPEA (6.49 mg, 50.2 μmol) were subsequently added. Compound 6 (15.1 mg, 25.0 μmol) was added, and the reaction mixture was stirred overnight at room temperature. The mixture was extracted with saturated brine (10 mL) and ethyl acetate. The organic phase was washed with deionized water (2×20 mL) and concentrated by evaporation under reduced pressure. Purification by silica gel chromatography (dichloromethane/methanol/aq. ammonia, v/v/v=90/10/1) yielded a colorless solid product (19.0 mg, 13.8 μmol, yield: 55.0%). This solid product was dissolved in trifluoroacetic acid (2 mL), stirred at room temperature for 1 hour, diluted with dichloromethane, and subjected to evaporation under reduced pressure to remove the organic solvent. Purification by semi-preparative HPLC (Phase A: 0.1% TFA in H O, Phase B: 0.1% TFA in CH CN. gradient: 0-20 min, 5-100% B. flow rate: 4 mL/min. UV: 280 nm) yielded NI-HBED-CC-PSMA-11 as a white solid product (3.5 mg, 3.18 μmol, yield: 54.8%).

### Structural Confirmation of NI-HBED-CC-PSMA-11

HRMS: m/z calcd for C₅₀H₇₁NO [M+H]⁺: 1099.4942, found: 1099.4940.

### Step 2: ⁶⁸Ga Labeling of the PSMA-Targeted Radioligand NI-HBED-CC-PSMA-11 Containing a Nitroaromatic Heterocyclic Group

The ⁶⁸Ga labeling route is as follows.

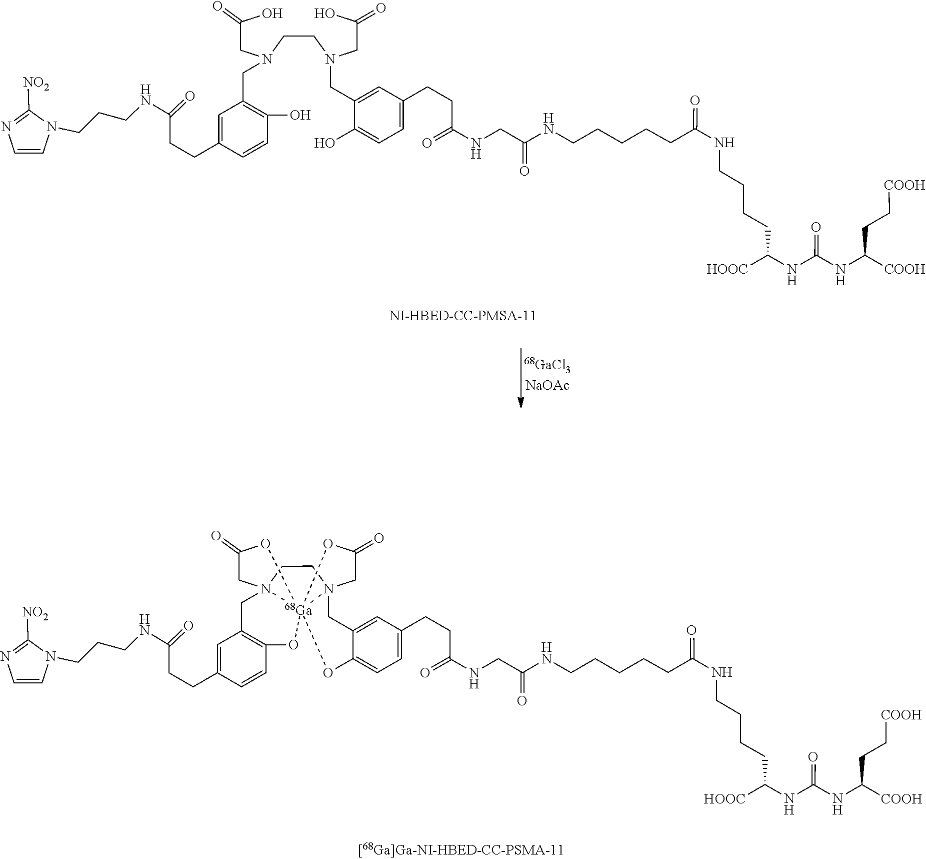

NI-HBED-CC-PSMA-11 (15 μg) obtained in Step 1 was dissolved in a sodium acetate buffer (135 μL, 3 M). The germanium-gallium generator (iThemba Laboratories, 740 MBq, 20 mCi) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 50° C. for 10 minutes, followed by cooling to room temperature. The radiochemical purity was determined by radio-HPLC equipped with a radioactive detector, confirming a radiochemical yield of >98% for [⁶⁸Ga]Ga-NI-HBED-CC-PSMA-11.

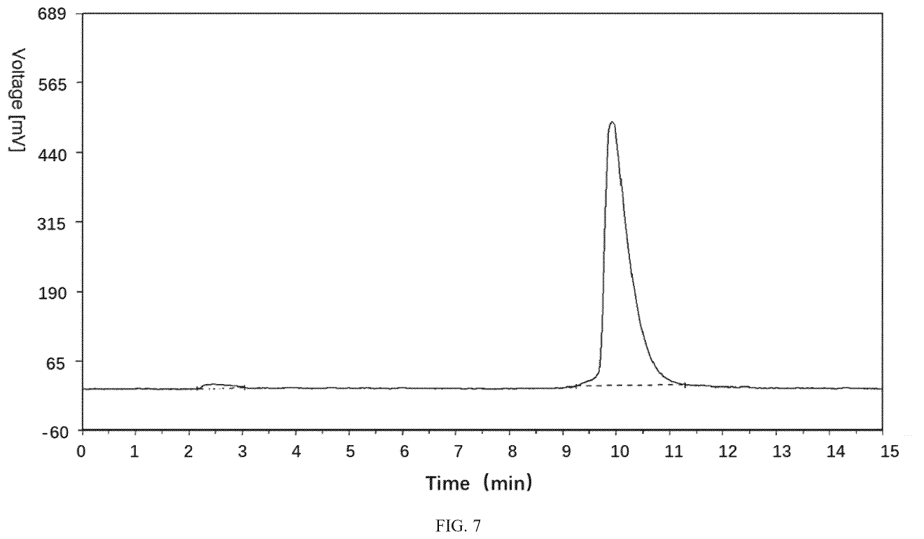

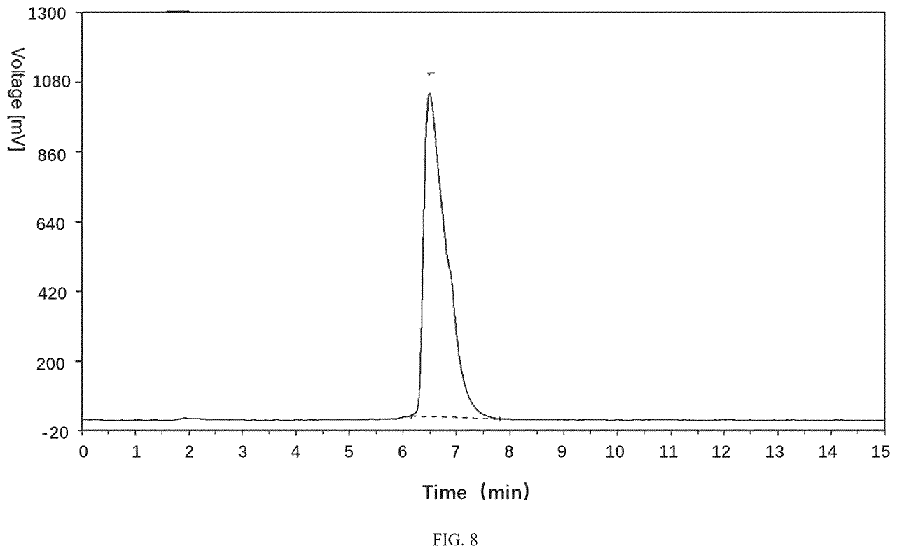

As shown in FIG. 8, FIG. 8 shows the radio-HPLC chromatogram of the labeling reaction mixture for [⁶⁸Ga]Ga-NI-HBED-CC-PSMA-11 prepared in Embodiment 8 according to the present disclosure, which demonstrates a radiochemical purity exceeding 98%.

### Embodiment 9: Preparation of [⁶⁸Ga]Ga-NI-DOTAGA-PSMA-11

### Step 1: Synthesis of the PSMA-Targeted Radioligand NI-DOTAGA-PSMA-11 Containing a Nitroaromatic Heterocyclic Group

The synthesis route is as follows.

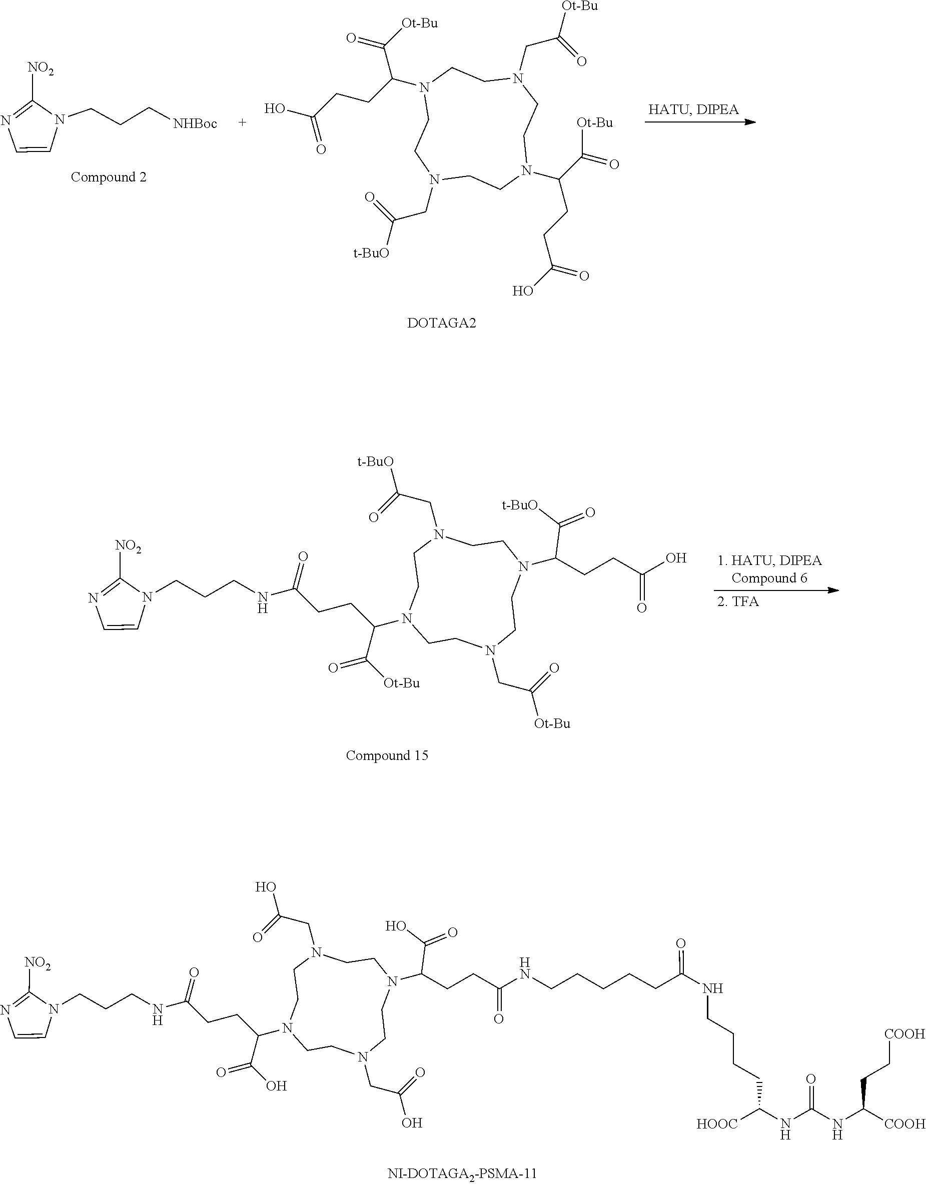 [SMILES: CC(C)(C)OC(=O)NCCCn1ccnc1[N+](=O)[O-] ; CC(C)(C)OC(=O)CN1CCN(C(CCC(=O)O)C(=O)OC(C)(C)C)CCN(CC(=O)OC(C)(C)C)CCN(C(CCC(=O)O)C(=O)OC(C)(C)C)CC1 ; CC(C)(C)OC(=O)CN1CCN(C(CCC(=O)O)C(=O)OC(C)(C)C)CCN(CC(=O)OC(C)(C)C)CCN(C(CCC(=O)NCCCn2ccnc2[N+](=O)[O-])C(=O)OC(C)(C)C)CC1 ; O=C(O)CC[C@H](NC(=O)N[C@@H](CCCCNC(=O)CCCCCNC(=O)CCC(C(=O)O)N1CCN(CC(=O)O)CCN(C(CCC(=O)NCCCn2ccnc2[N+](=O)[O-])C(=O)O)CCN(CC(=O)O)CC1)C(=O)O)C(=O)O]

Specifically, the following steps are included.

### (1) Synthesis of Compound 15

DOTAGA (200 mg, 0.259 mmol) was dissolved in anhydrous N,N-dimethylformamide (10 mL). Under an ice bath, HATU (141 mg, 0.372 mmol) and DIPEA (66.8 mg, 0.518 mmol) were added sequentially. Compound 2 was dissolved in trifluoroacetic acid (TFA, 2 mL) and stirred for 1 hour, and then subjected to evaporation under reduced pressure to yield a white solid intermediate. This intermediate (44.1 mg, 0.259 mmol) in anhydrous N,N-dimethylformamide (5 mL) was added dropwise to the above reaction mixture and stirred at room temperature for 4 hours. The mixture was extracted with saturated brine (30 mL) and ethyl acetate. The organic phase was washed with deionized water (2×30 mL), and concentrated by evaporation under reduced pressure. Purification by silica gel column chromatography (dichloromethane/methanol/aq. ammonia, v/v/v=90/10/1) yielded Compound 15 as a colorless product (86.5 mg, 0.0935 mmol, yield: 36.1%).

### Structural Confirmation of Compound 15

HRMS: m/z calcd for CH₇₇N₈O [M+H]⁺: 925.5605, found: 925.5600.

### (6) Synthesis of NI-DOTAGA-PSMA-11

Compound 15 (25.6 mg, 27.6 μmol) was dissolved in anhydrous N,N-dimethylformamide (5 mL). Under an ice bath, HATU (14.3 mg, 30.1 μmol) and DIPEA (6.49 mg, 50.2 μmol) were subsequently added. Compound 6 (15.1 mg, 25.0 μmol) was added, and the reaction mixture was stirred overnight at room temperature. The mixture was extracted with saturated brine (10 mL) and ethyl acetate. The organic phase was washed with deionized water (2×20 mL) and concentrated by evaporation under reduced pressure. Purification by silica gel chromatography (dichloromethane/methanol/aq. ammonia, v/v/v=90/10/1) yielded a colorless solid product. This solid product was dissolved in TFA (2 mL), stirred at room temperature for 1 hour, diluted with dichloromethane, and subjected to evaporation under reduced pressure to remove the organic solvent. Purification by semi-preparative HPLC (Phase A: 0.1% TFA in H O, Phase B: 0.1% TFA in CH CN. gradient: 0-20 min, 5-100% B. flow rate: 4 mL/min. UV: 280 nm) yielded NI-DOTAGA-PSMA-11 as a white solid product (4.8 mg, 3.18 μmol, yield: 54.8%).

### Structural Confirmation of NI-DOTAGA-PSMA-11

HRMS: m/z calcd for CH₇₅NO [M+H]⁺: 1115.5215, found: 1115.5210.

### Step 2: ⁶⁸Ga Labeling of the PSMA-Targeted Radioligand NI-DOTAGA-PSMA-11 Containing a Nitroaromatic Heterocyclic Group

The ⁶⁸Ga labeling route is as follows.

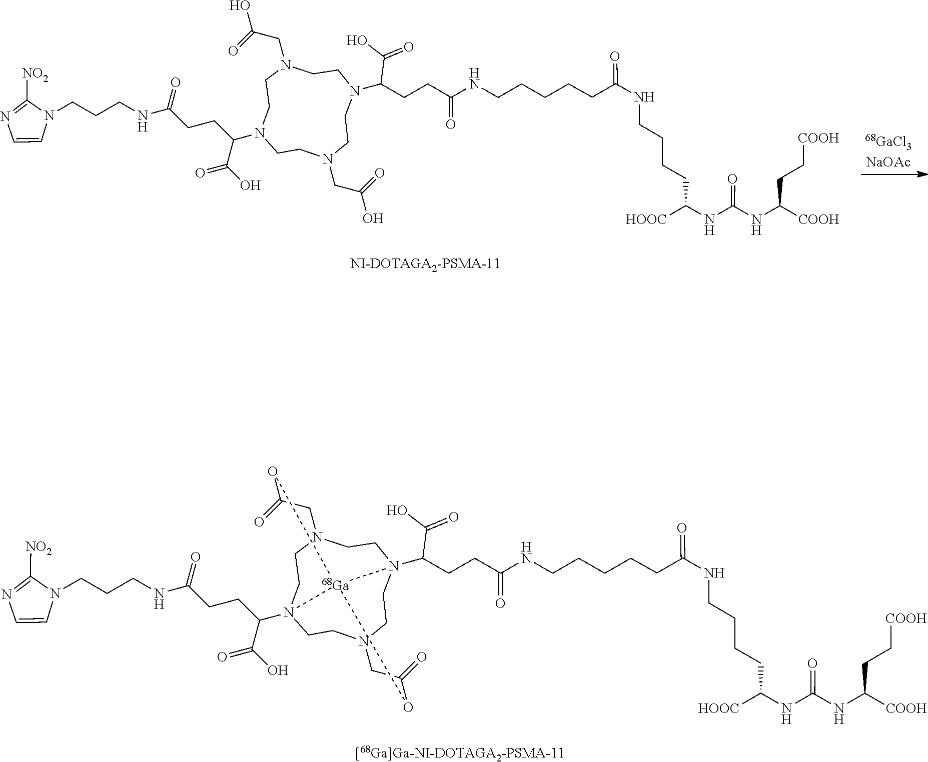

NI-DOTAGA-PSMA-11 (15 μg) obtained in Step 1 was dissolved in a sodium acetate buffer (135 μL, 3 M). The germanium-gallium generator (iThemba Laboratories, 740 MBq, 20 mCi) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 50° C. for 10 minutes, followed by cooling to room temperature. The radiochemical purity was determined by radio-HPLC equipped with a radioactive detector, confirming a radiochemical yield of >98% for [⁶⁸Ga]Ga-NI-DOTAGA2-PSMA-11.

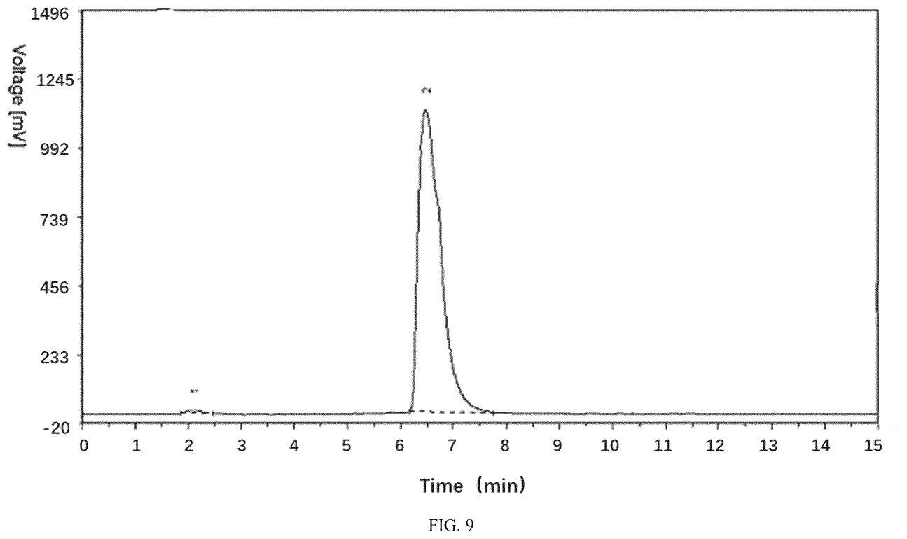

As shown in FIG. 9, FIG. 9 shows the radio-HPLC chromatogram of the labeling reaction mixture for [⁶⁸Ga]Ga-NI-DOTAGA2-PSMA-11 prepared in Embodiment 9 according to the present disclosure, which demonstrates a radiochemical purity exceeding 98%.

### Embodiment 10: Preparation of [⁶⁸Ga]Ga-NI-HBED-CC-PSMA-093

### Step 1: Synthesis of the PSMA-Targeted Radioligand NI-HBED-CC-PSMA-093 Containing a Nitroaromatic Heterocyclic Group

The synthesis route is as follows.

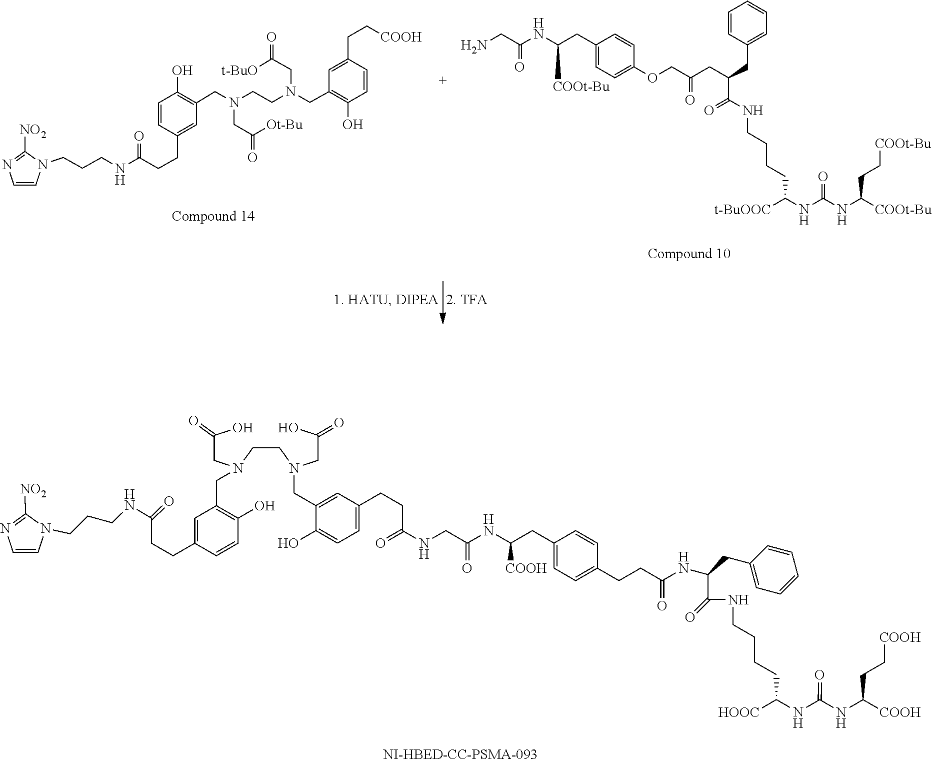

Compound 14 (20.1 mg, 25.1 μmol) was dissolved in anhydrous N,N-dimethylformamide (5 mL). Under an ice bath, HATU (14.3 mg, 30.1 μmol) and DIPEA (6.49 mg, 50.2 μmol) were added sequentially. Compound 10 (29.2 mg, 30.1 μmol) was added, and the reaction mixture was stirred overnight at room temperature. The mixture was extracted with saturated brine (10 mL) and ethyl acetate. The organic phase was washed with deionized water (2×20 mL), dried, and concentrated by evaporation under reduced pressure. Purification by silica gel chromatography (dichloromethane/methanol/aq. ammonia, v/v/v=90/10/1) yielded a colorless solid product (16.8 mg, 9.61 μmol, yield: 38.3%). This solid product (11.3 mg, 6.47 μmol) was dissolved in trifluoroacetic acid (TFA, 2 mL), stirred at room temperature for 1 hour, diluted with dichloromethane, and subjected to evaporation under reduced pressure to remove the organic solvent. Purification by semi-preparative HPLC (Phase A: 0.1% TFA in H O, Phase B: 0.1% TFA in CH CN. gradient: 0-20 min, 5-100% B. flow rate: 4 mL/min. UV: 280 nm) yielded NI-HBED-CC-PSMA-093 as a white solid product (4.40 mg, 3.31 μmol, yield: 51.1%).

### Structural Confirmation of NI-HBED-CC-PSMA-093

HRMS: m/z calcd for C₆₆H₈₃NO [M+H]⁺: 1411.5688, found: 1411.5703.

### Step 2: ⁶⁸Ga Labeling of the PSMA-Targeted Radioligand NI-HBED-CC-PSMA-093 Containing a Nitroaromatic Heterocyclic Group

The ⁶⁸Ga labeling route is as follows.

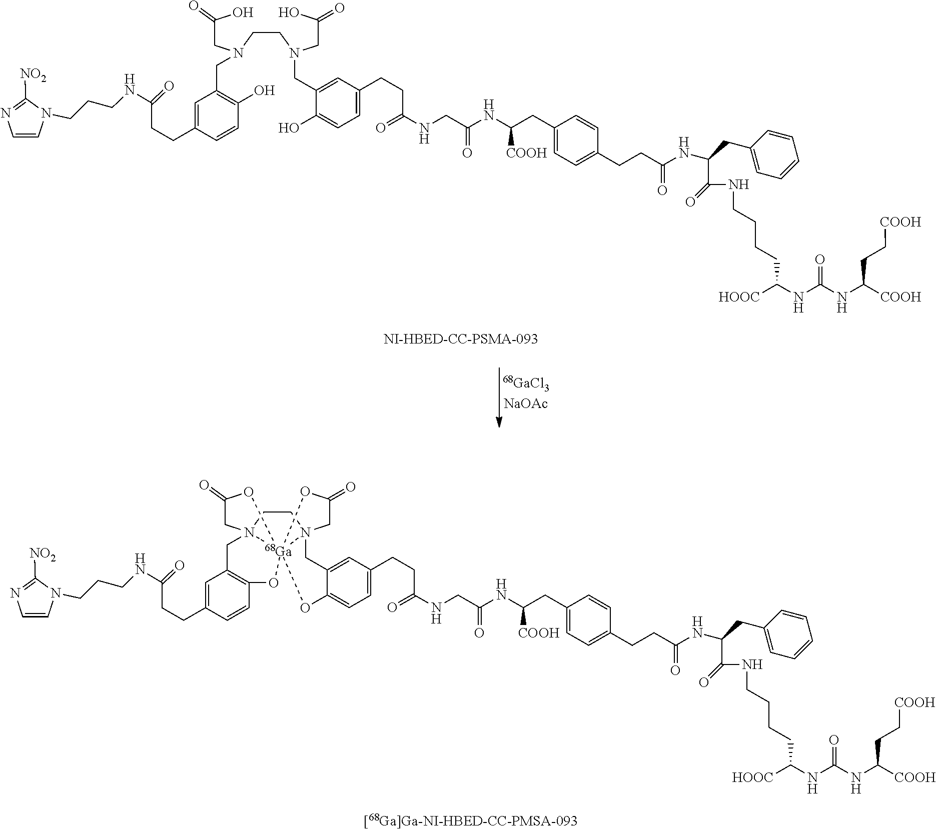

NI-HBED-CC-PSMA-093 (15 μg) obtained in Step 1 was dissolved in a sodium acetate buffer (135 μL, 3 M). The germanium-gallium generator (iThemba Laboratories, 740 MBq, 20 mCi) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 50° C. for 10 minutes, followed by cooling to room temperature. The radiochemical purity was determined by radio-HPLC equipped with a radioactive detector, confirming a radiochemical yield of >98% for [⁶⁸Ga]Ga-NI-HBED-CC-PSMA-093.

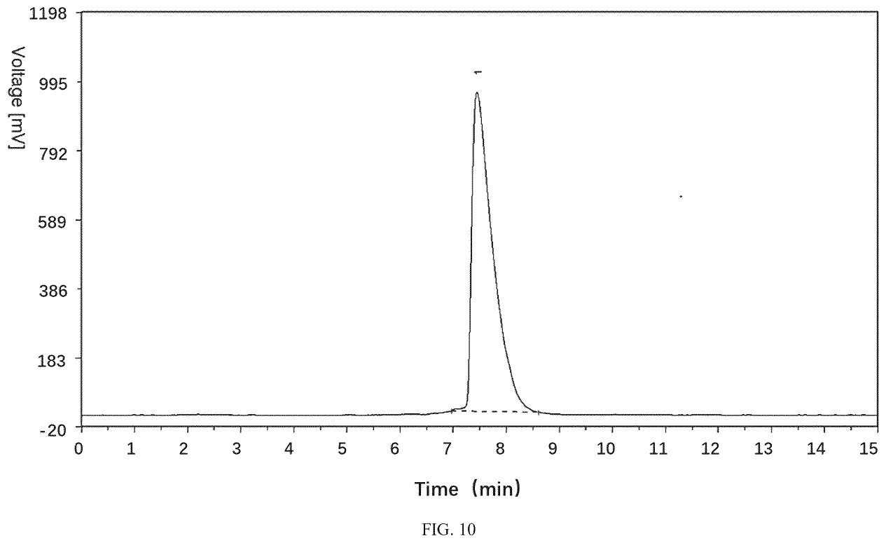

As shown in FIG. 10, FIG. 10 shows the radio-HPLC chromatogram of the labeling reaction mixture for [⁶⁸Ga]Ga-NI-HBED-CC-PSMA-093 prepared in Embodiment 10 according to the present disclosure, which demonstrates a radiochemical purity exceeding 98%.

### Embodiment 11: Preparation of [⁶⁸Ga]Ga-NI-DOTAGA-PSMA-093

### Step 1: Synthesis of the PSMA-Targeted Radioligand NI-DOTAGA-PSMA-093 Containing a Nitroaromatic Heterocyclic Group

The synthesis route is as follows.

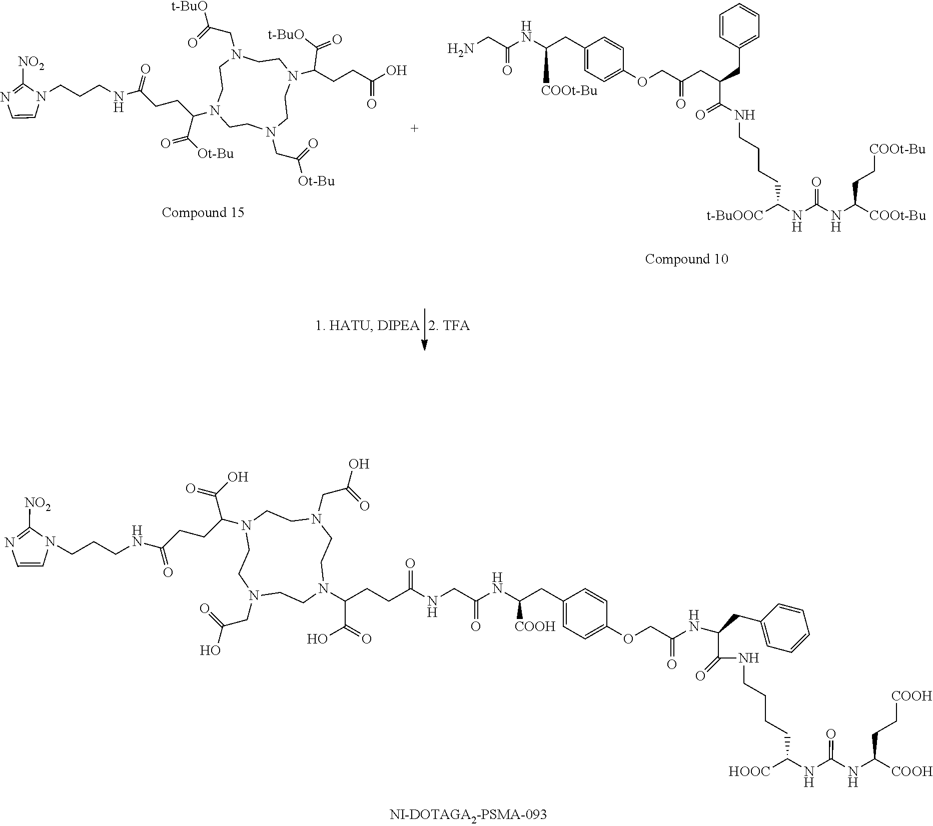

Compound 15 (23.2 mg, 25.1 μmol) was dissolved in anhydrous N,N-dimethylformamide (5 mL). Under an ice bath, HATU (14.3 mg, 30.1 μmol) and DIPEA (6.49 mg, 50.2 μmol) were added sequentially. Compound 10 (29.2 mg, 30.1 μmol) was added, and the reaction mixture was stirred overnight at room temperature. The mixture was extracted with saturated brine (10 mL) and ethyl acetate. The organic phase was washed with deionized water (2×20 mL), dried, and concentrated by evaporation under reduced pressure. Purification by silica gel column chromatography (dichloromethane/methanol/aq. ammonia, v/v/v=90/10/1) yielded a colorless solid product (18.0 mg, 9.61 μmol, yield: 38.3%). This solid product (11.3 mg, 6.01 μmol, 1 eq.) was dissolved in trifluoroacetic acid (TEA, 2 mL), stirred at room temperature for 1 hour, diluted with dichloromethane, and subjected to evaporation under reduced pressure to remove the organic solvent. Purification by semi-preparative HPLC (Phase A: 0.1% TFA in H O, Phase B: 0.1% TFA in CH CN. gradient: 0-20 min, 5-100% B. flow rate: 4 mL/min. UV: 280 nm) yielded NI-DOTAGA-PSMA-093 as a white solid product (3.98 mg, 2.79 μmol, yield: 46.4%).

### Structural Confirmation of NI-DOTAGA-PSMA-093

HRMS: m/z calcd for C₆₂H₈₇NO [M+H]⁺: 1427.5961, found: 1427.5956.

### Step 2: ⁶⁸Ga Labeling of the PSMA-Targeted Radioligand NI-DOTAGA-PSMA-093 Containing a Nitroaromatic Heterocyclic Group

The ⁶⁸Ga labeling route is as follows.

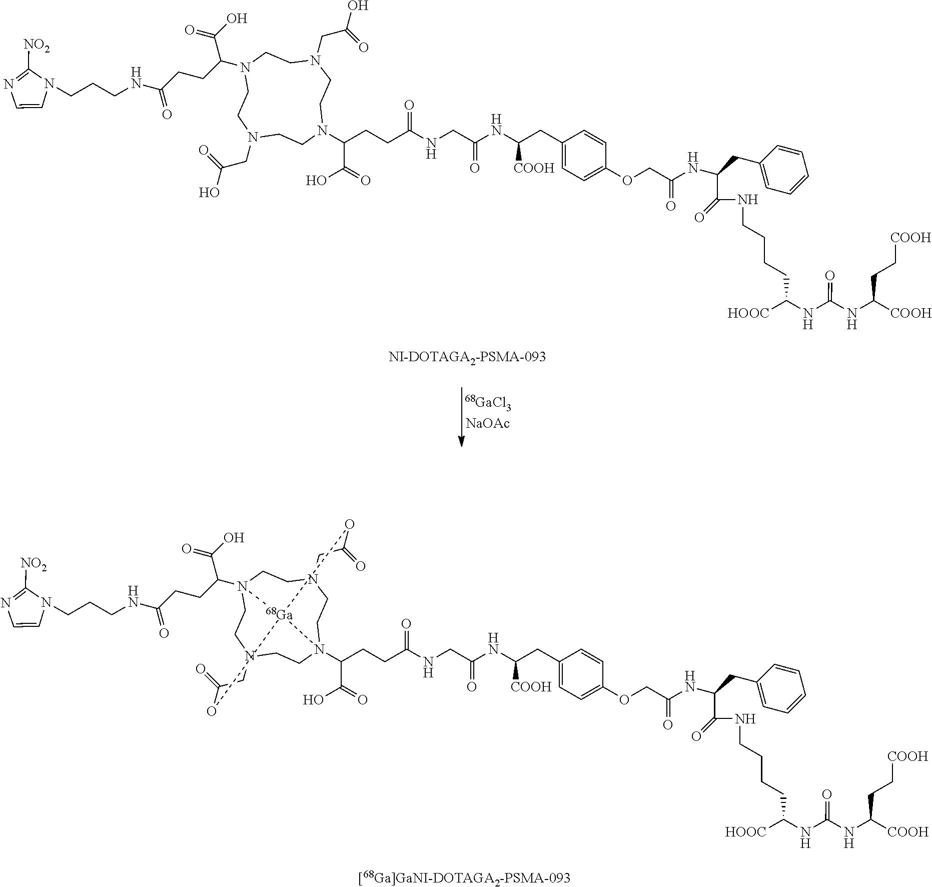

NI-DOTAGA-PSMA-093 (15 μg) obtained in Step 1 was dissolved in a sodium acetate buffer (135 μL, 3 M). The germanium-gallium generator (iThemba Laboratories, 740 MBq, 20 mCi) was eluted with high-purity hydrochloric acid (6 mL, 0.6 M) to obtain a [⁶⁸Ga]GaCl₃ hydrochloric acid solution. A 300 μL aliquot of this solution was added to the mixture of the radioligand and sodium acetate. After thorough mixing, the reaction proceeded at 50° C. for 10 minutes, followed by cooling to room temperature. The radiochemical purity was determined by radio-HPLC equipped with a radioactive detector, confirming a radiochemical yield of >98% for [⁶⁸Ga]Ga-NI-DOTAGA-PSMA-093.

As shown in FIG. 11, FIG. 11 shows the radio-HPLC chromatogram of the labeling reaction mixture for [⁶⁸Ga]Ga-NI-DOTAGA2-PSMA-093 prepared in Embodiment 11 according to the present disclosure, which demonstrates a radiochemical purity exceeding 98%.

### Application Embodiment 1

In vitro uptake of [⁶⁸Ga]Ga-AAZTA-NI-PSMA and [⁶⁸Ga]Ga-AAZTA-NI-PSMA-11 in 22RV1-FOLH1-oe Cells.

22RV1-FOLH1-oe cells were suspended at a density of 1×10⁵ cells/mL. A 500 μL aliquot was added to each well of six 12-well plates. Each group of wells was treated with 37 KBq of [⁶⁸Ga]Ga-AAZTA-NI-PSMA, [⁶⁸Ga]Ga-AAZTA-NI-PSMA-11, and [⁶⁸Ga]Ga-HBED-CC-PSMA-11 solution. Incubation proceeded at 37° C. At 15, 30, 60, 90, and 120 minutes, uptake was terminated with PBS. Cells were lysed with 1 M NaOH, and lysates were absorbed onto filter paper for radioactivity measurement in plastic tubes. Blocking was performed at 60 minutes using excess unlabeled PSMA-11. After incubation, uptake was terminated with PBS, cells were lysed with 1 M NaOH, and lysates were absorbed onto filter paper for radioactivity measurement in plastic tubes.

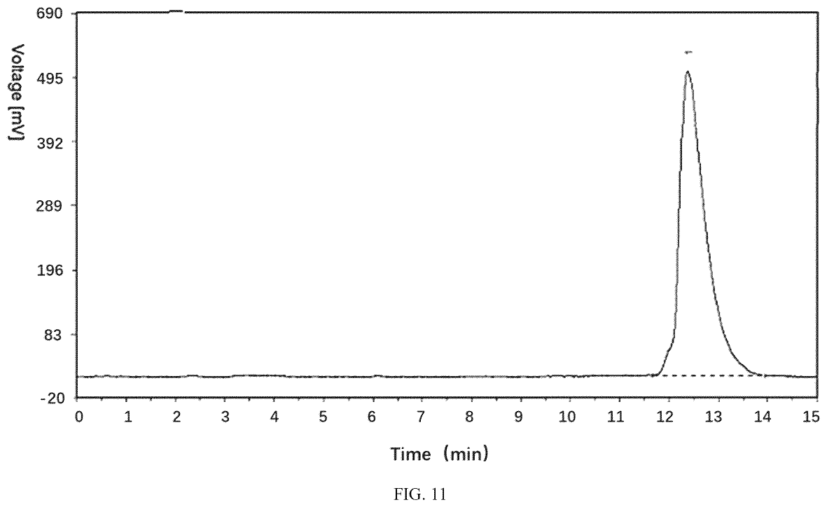

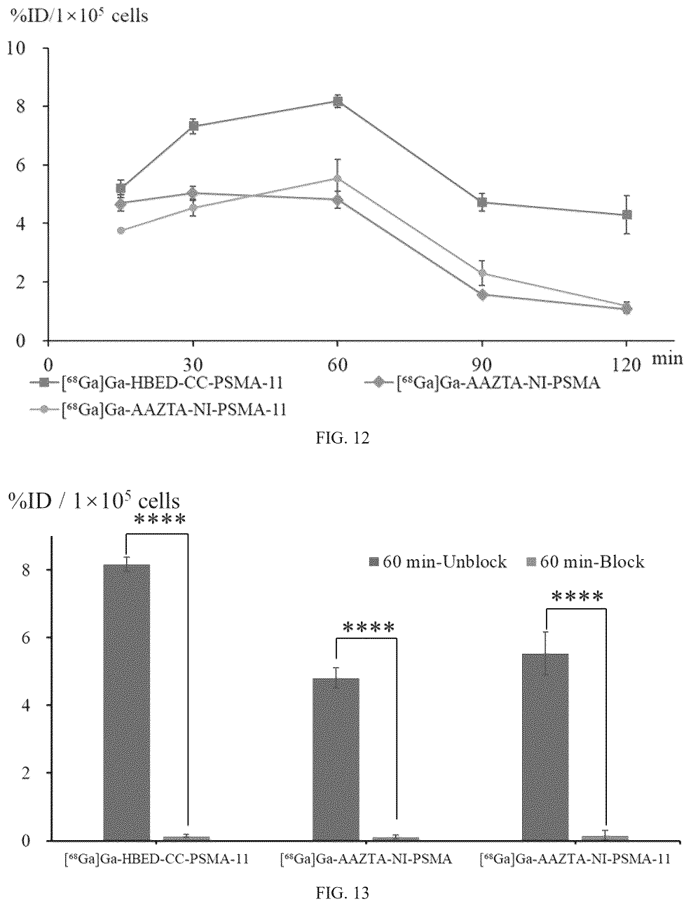

FIG. 12 shows the uptake-time profile (n=3) of [⁶⁸Ga]Ga-AAZTA-NI-PSMA, [⁶⁸Ga]Ga-AAZTA-NI-PSMA-11, and [⁶⁸Ga]Ga-HBED-CC-PSMA-11 in in vitro 22RV1-FOLH1-oe cells in Application Embodiment 1 according to the present disclosure.

FIG. 13 shows the specific binding analysis (n=3) of [⁶⁸Ga]Ga-AAZTA-NI-PSMA, [⁶⁸Ga]Ga-AAZTA-NI-PSMA-11, and [⁶⁸Ga]Ga-HBED-CC-PSMA-11 to prostate-specific membrane antigen receptors in in vitro 22RV1-FOLH1-oe cells in Application Embodiment 1 according to the present disclosure.

Results from the in vitro cell uptake experiments demonstrate that in 22RV1-FOLH1-oe cells, the uptake of the radioactive metal complexes [⁶⁸Ga]Ga-AAZTA-NI-PSMA, [⁶⁸Ga]Ga-AAZTA-NI-PSMA-11, and [⁶⁸Ga]Ga-HBED-CC-PSMA-11 initially increased and subsequently decreased with prolonged incubation time. Among these, the cellular uptake of [⁶⁸Ga]Ga-HBED-CC-PSMA-11 was significantly higher than that of the other radioactive metal complexes at all time points. In the presence of excess non-radiolabeled PSMA-11, the uptake of all radioactive metal complexes was markedly reduced, indicating that the uptake of the complexes by 22RV1-FOLH1-oe cells is specific, and the binding of PSMA to the radioactive metal complexes of the present disclosure is specific.

### Application Embodiment 2

In vitro uptake of [⁶⁸Ga]Ga-AAZTA-NI-PSMA, [⁶⁸Ga]Ga-DOTA-NI-PSMA, and [⁶⁸Ga]Ga-HBED-CC-NI-PSMA in 22RV1-FOLH1-oe Cells.

22RV1-FOLH1-oe cells were suspended at a density of 1.5×10⁵ cells/mL. A 500 μL aliquot was added to each well of six 12-well plates. Each group of wells was treated with 37 KBq of [⁶⁸Ga]Ga-AAZTA-NI-PSMA, [⁶⁸Ga]Ga-DOTA-NI-PSMA, [⁶⁸Ga]Ga-HBED-CC-NI-PSMA, and [⁶⁸Ga]Ga-HBED-CC-PSMA-11 solution. Incubation proceeded at 37° C. At 15, 30, 60, 90, and 120 minutes, uptake was terminated with PBS. Cells were lysed with 1 M NaOH, and lysates were absorbed onto filter paper for radioactivity measurement in plastic tubes. Blocking was performed at 60 minutes using excess unlabeled PSMA-11. After incubation, uptake was terminated with PBS, cells were lysed with 1 M NaOH, and lysates were absorbed onto filter paper for radioactivity measurement in plastic tubes.

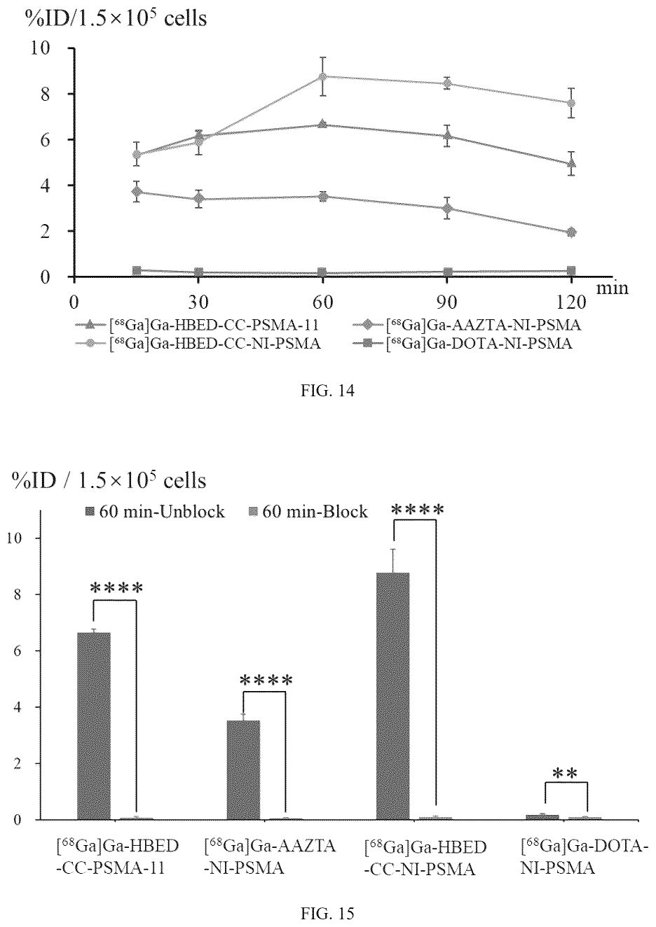

FIG. 14 shows the uptake-time profile (n=3) of [⁶⁸Ga]Ga-AAZTA-NI-PSMA, [⁶⁸Ga]Ga-DOTA-NI-PSMA, [⁶⁸Ga]Ga-HBED-CC-NI-PSMA, and [⁶⁸Ga]Ga-HBED-CC-PSMA-11 in in vitro 22RV1-FOLH1-oe cells in Application Embodiment 2 according to the present disclosure.

FIG. 15 shows the specific binding analysis (n=3) of [⁶⁸Ga]Ga-AAZTA-NI-PSMA, [⁶⁸Ga]Ga-DOTA-NI-PSMA, [⁶⁸Ga]Ga-HBED-CC-NI-PSMA, and [⁶⁸Ga]Ga-HBED-CC-PSMA-11 to prostate-specific membrane antigen receptors in in vitro 22RV1-FOLH1-oe cells in Application Embodiment 2 according to the present disclosure.

Results from the in vitro cell uptake experiments demonstrate that in 22RV1-FOLH1-oe cells, the uptake of the radioactive metal complexes [⁶⁸Ga]Ga-AAZTA-NI-PSMA, [⁶⁸Ga]Ga-DOTA-NI-PSMA, [⁶⁸Ga]Ga-HBED-CC-NI-PSMA, and [⁶⁸Ga]Ga-HBED-CC-PSMA-11 initially increased and subsequently decreased with prolonged incubation time. Among these, the cellular uptake of [⁶⁸Ga]Ga-HBED-CC-NI-PSMA was significantly higher than that of the FDA-approved [⁶⁸Ga]Ga-HBED-CC-PSMA-11 at multiple time points. In the presence of excess non-radiolabeled PSMA-11, the uptake of all radioactive metal complexes was markedly reduced, indicating that the uptake of the complexes by 22RV1-FOLH1-oe cells is specific, and the binding of PSMA to the radioactive metal complexes of the present disclosure is specific.

### Application Embodiment 3

In vitro uptake of [⁶⁸Ga]Ga-AAZTA-NI-PSMA-093 in 22RV1-FOLH1-oe cells.

22RV1-FOLH1-oe cells were suspended at a density of 1×10⁵ cells/mL. A 500 μL aliquot was added to each well of three 12-well plates. Each group of wells was treated with 37 KBq of [⁶⁸Ga]Ga-AAZTA-NI-PSMA-093 and [⁶⁸Ga]Ga-HBED-CC-PSMA-11 solution. Incubation proceeded at 37° C. At 15, 30, 60, 90, and 120 minutes, uptake was terminated with PBS. Cells were lysed with 1 M NaOH, and lysates were absorbed onto filter paper for radioactivity measurement in plastic tubes. Blocking was performed at 60 minutes using excess unlabeled PSMA-11. After incubation, uptake was terminated with PBS, cells were lysed with 1 M NaOH, and lysates were absorbed onto filter paper for radioactivity measurement in plastic tubes.

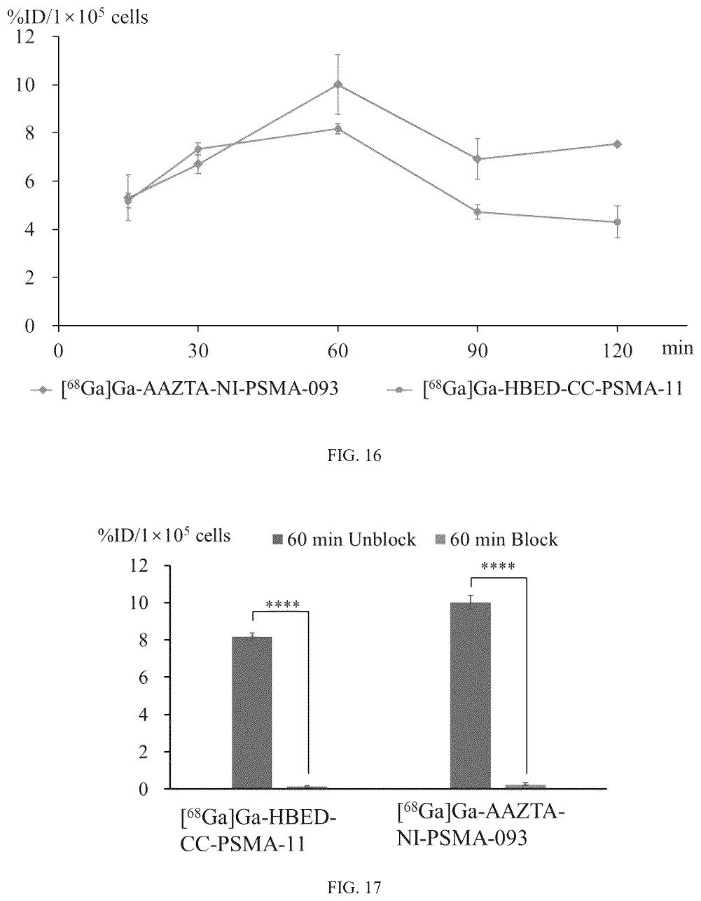

FIG. 16 shows the uptake-time profile (n=3) of [⁶⁸Ga]Ga-AAZTA-NI-PSMA-093 and [⁶⁸Ga]Ga-HBED-CC-PSMA-11 in in vitro 22RV1-FOLH1-oe cells in Application Embodiment 3 according to the present disclosure.

FIG. 17 shows the specific binding analysis (n=3) of [⁶⁸Ga]Ga-AAZTA-NI-PSMA-093 and [⁶⁸Ga]Ga-HBED-CC-PSMA-11 to prostate-specific membrane antigen receptors in in vitro 22RV1-FOLH1-oe cells in Application Embodiment 3 according to the present disclosure.

Results from the in vitro cell uptake experiments demonstrate that in 22RV1-FOLH1-oe cells, the uptake of the radioactive metal complexes [⁶⁸Ga]Ga-HBED-CC-PSMA-11 and [⁶⁸Ga]Ga-AAZTA-NI-PSMA-093 initially increased and subsequently decreased with prolonged incubation time. Among these, the cellular uptake of [⁶⁸Ga]Ga-AAZTA-NI-PSMA-093 was significantly higher than that of the FDA-approved [⁶⁸Ga]Ga-HBED-CC-PSMA-11 at multiple time points. In the presence of excess non-radiolabeled PSMA-11, the uptake of all radioactive metal complexes was markedly reduced, indicating that the uptake of the complexes by 22RV1-FOLH1-oe cells is specific, and the binding of PSMA to the radioactive metal complexes of the present disclosure is specific.

### Application Embodiment 4

In vitro uptake of [⁶⁸Ga]Ga-NI-HBED-CC-PSMA-11 and [⁶⁸Ga]Ga-NI-HBED-CC-PSMA-093 in 22RV1-FOLH1-oe cells.

22RV1-FOLH1-oe cells were suspended at a density of 2×10⁵ cells/mL. A 500 μL aliquot was added to each well of six 12-well plates. Each group of wells was treated with 37 KBq of the following solutions: [⁶⁸Ga]Ga-HBED-CC-PSMA-11, [⁶⁸Ga]Ga-NI-HBED-CC-PSMA-11, [⁶⁸Ga]Ga-HBED-CC-PSMA-093, and [⁶⁸Ga]Ga-NI-HBED-CC-PSMA-093. Incubation proceeded at 37° C. At 15, 30, 60, 90, and 120 minutes, uptake was terminated with PBS. Cells were lysed with 1 M NaOH, and lysates were absorbed onto filter paper for radioactivity measurement in plastic tubes. Blocking was performed at 60 minutes using excess unlabeled PSMA-11. After incubation, uptake was terminated with PBS, cells were lysed with 1 M NaOH, and lysates were absorbed onto filter paper for radioactivity measurement in plastic tubes.

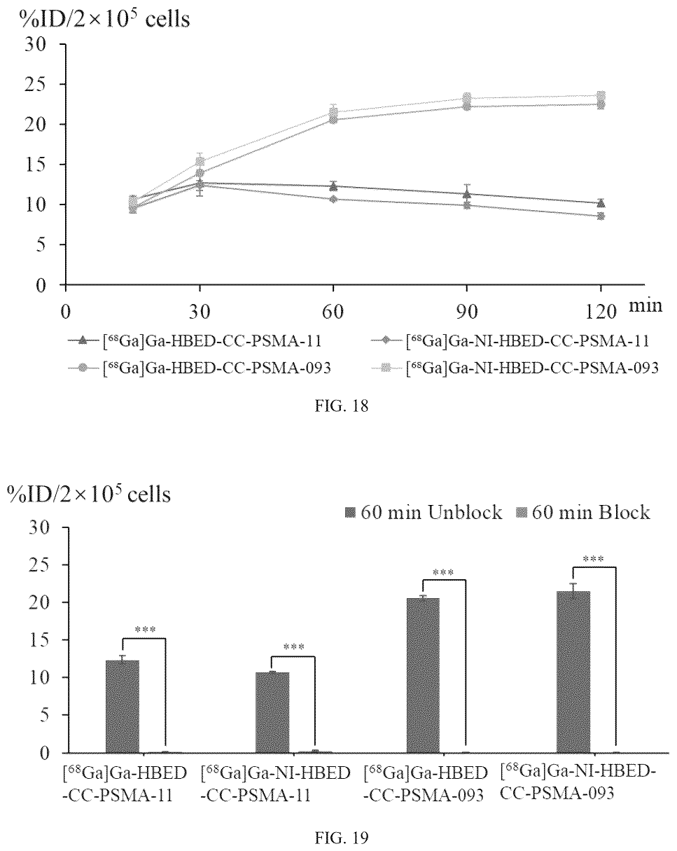

FIG. 18 shows the uptake-time profile (n=3) of [⁶⁸Ga]Ga-NI-HBED-CC-PSMA-11, [⁶⁸Ga]Ga-HBED-CC-PSMA-11, [⁶⁸Ga]Ga-NI-HBED-CC-PSMA-093, and [⁶⁸Ga]Ga-HBED-CC-PSMA-093 in in vitro 22RV1-FOLH1-oe cells in Application Embodiment 4 according to the present disclosure.

FIG. 19 shows the specific binding analysis (n=3) of [⁶⁸Ga]Ga-NI-HBED-CC-PSMA-11, [⁶⁸Ga]Ga-HBED-CC-PSMA-11, [⁶⁸Ga]Ga-NI-HBED-CC-PSMA-093, and [⁶⁸Ga]Ga-HBED-CC-PSMA-093 to prostate-specific membrane antigen receptors in in vitro 22RV1-FOLH1-oe cells in Application Embodiment 4 according to the present disclosure.

Results from the in vitro cell uptake experiments demonstrate that in 22RV1-FOLH1-oe cells, the uptake of the radioactive metal complexes [⁶⁸Ga]Ga-HBED-CC-PSMA-11, [⁶⁸Ga]Ga-NI-HBED-CC-PSMA-11, [⁶⁸Ga]Ga-HBED-CC-PSMA-093, and [⁶⁸Ga]Ga-NI-HBED-CC-PSMA-093 gradually increased with prolonged incubation time. Among these, the cellular uptake of [⁶⁸Ga]Ga-HBED-CC-PSMA-093 was significantly higher than those of other radioactive metal complexes at all time points. In the presence of excess non-radiolabeled PSMA-11, the uptake of all radioactive metal complexes was markedly reduced, indicating that the uptake of the complexes by 22RV1-FOLH1-oe cells is specific, and the binding of PSMA to the radioactive metal complexes of the present disclosure is specific.

The present disclosure provides radioligands for a PSMA-targeted radioactive metal complex containing a nitroaromatic heterocyclic group, which include different chelators capable of chelating nearly all clinically used diagnostic and therapeutic radionuclides. N,N′-Bis[2-hydroxy-5-(carboxyethyl)benzyl]ethylenediamine-N,N′-diacetic acid (HBED-CC) exhibits a high thermodynamic stability constant with Ga³⁺ (logK_{ML}: 38.5) and requires low coordination energy; therefore, labeling of [⁶⁸Ga]Ga-HBED-CC is rapid and efficient. 1,4,7,10-Tetraazacyclododecane-1,4,7,10-tetraacetic acid (DOTA), 2,2′-(4,10-bis(carboxymethyl)-1,4,7,10-tetraazacyclododecane-1,7-diyl)diacetic acid (DOTAGA2), and 2,2′-(6-(bis(carboxymethyl)amino)-6-(4-carboxybutyl)-1,4-diazepane-1,4-diacetic acid (AAZTA) enable labeling with multiple medical radionuclides, including: β⁺-emitting diagnostic nuclides: ⁶⁸Ga, [¹⁸F]AlF, ⁴⁴Sc, ⁶⁴Cu; β⁻-emitting therapeutic nuclides: ¹⁷⁷Lu, ⁹⁰Y; and α-emitting therapeutic nuclides: ²²⁵Ac, ^{212/213}Bi. Among these, AAZTA achieves radionuclide labeling under milder conditions compared to DOTA.

The PSMA-targeted radioactive metal complex containing a nitroaromatic heterocyclic group of the present disclosure includes a PSMA-targeted Lys-CO-Glu structure. It binds to tumor cells overexpressing PSMA and is co-internalized with PSMA receptors into cells. The nitroaromatic heterocyclic group enhances the retention of the complex within tumor cells.

In the present disclosure, [⁶⁸Ga]Ga-NI-HBED-CC-PSMA-093, [⁶⁸Ga]Ga-AAZTA-NI-PSMA-093, and [⁶⁸Ga]Ga-HBED-CC-NI-PSMA exhibit significantly higher uptake in PSMA-positive cells than the FDA-approved [⁶⁸Ga]Ga-HBED-CC-PSMA-11.

The PSMA-targeted radioactive metal ligand containing a nitroaromatic heterocyclic group of the present disclosure enables labeling with multiple diagnostic and therapeutic radionuclides. When labeled with imaging radionuclides, it serves as an imaging radioactive metal ligand to obtain high-contrast images. When labeled with therapeutic radionuclides, it serves as a radionuclide therapeutic agent to increase drug retention at tumor sites and improve treatment efficacy. Therefore, the proposed PSMA-targeted radioactive metal ligand better achieves integrated diagnosis and treatment for PSMA receptor-positive tumors.

The above descriptions represent some embodiments of the present disclosure but do not limit the scope of its implementation. Equivalent modifications or variations made based on the claims and specification content of the present disclosure shall remain within the scope of the present disclosure.

1. A prostate-specific membrane antigen (PSMA)-targeted radioactive metal ligand containing a nitroaromatic heterocyclic group, represented by Formula I-1: 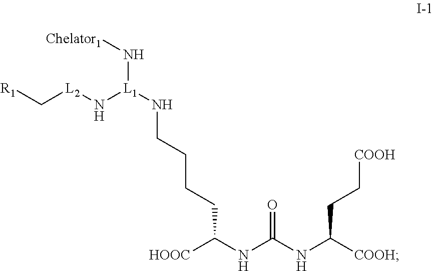
wherein, Chelator₁ is a bifunctional chelator structure selected from the group consisting of: 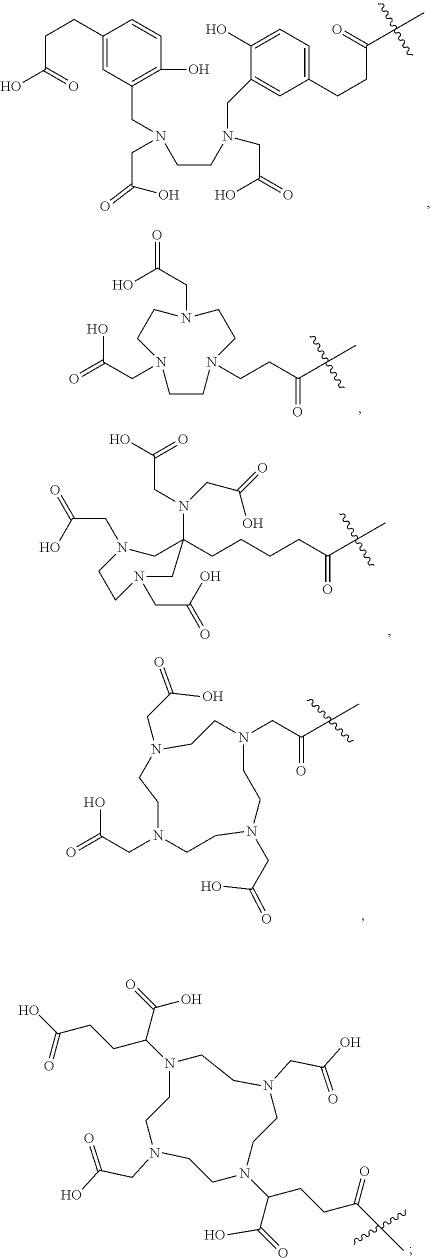
R₁ is a nitroaromatic heterocyclic or nitrophenyl group selected from the group consisting of: 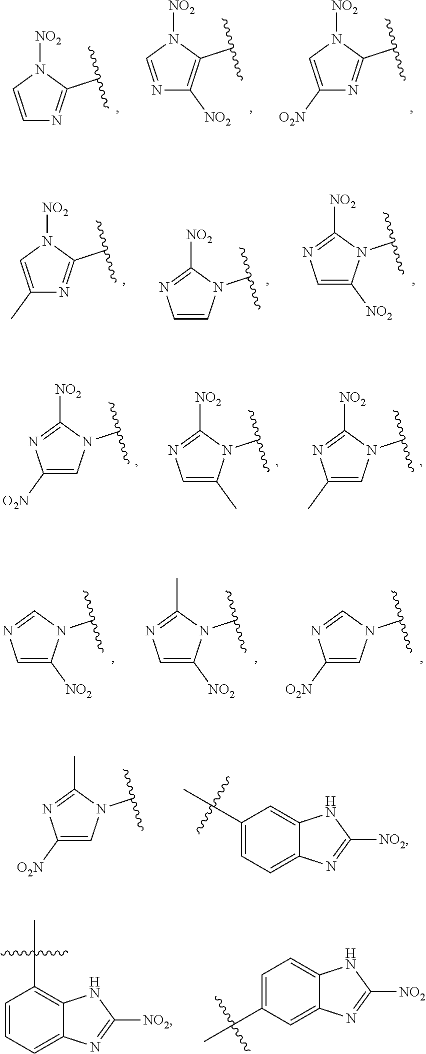 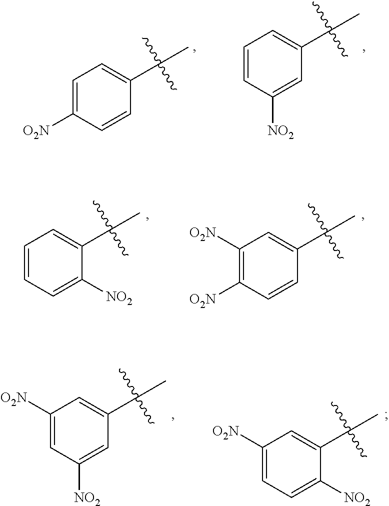 [SMILES: *c1ccc([N+](=O)[O-])cc1 ; *c1cccc([N+](=O)[O-])c1 ; *c1ccccc1[N+](=O)[O-] ; *c1ccc([N+](=O)[O-])c([N+](=O)[O-])c1 ; *c1cc([N+](=O)[O-])cc([N+](=O)[O-])c1 ; *c1cc([N+](=O)[O-])ccc1[N+](=O)[O-]]
L₁ is a linker group between the Chelator₁ and a PSMA-targeted group, selected from the group consisting of: 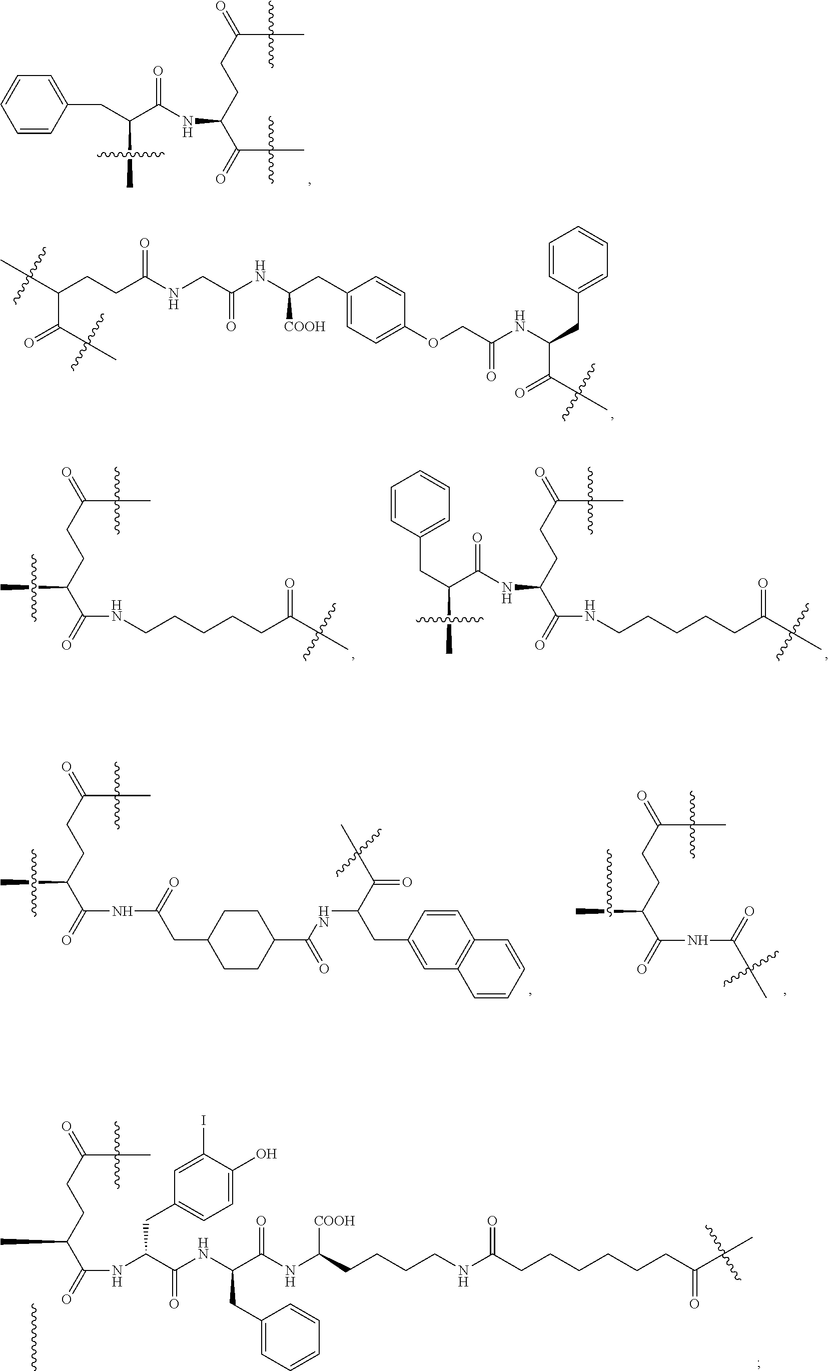
L₂ is a linker group between the Chelator₁ and the R₁, selected from the group consisting of: 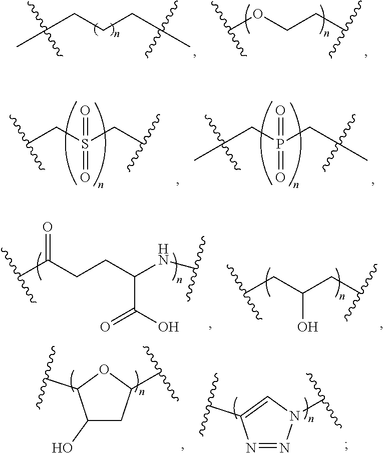
wherein n is an integer in a range from 0 to 6.

2. A prostate-specific membrane antigen (PSMA)-targeted radioactive metal ligand containing a nitroaromatic heterocyclic group, represented by Formula II-1: 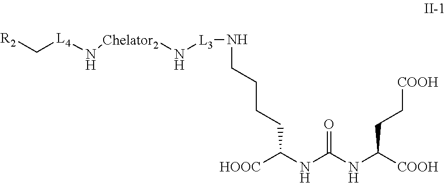
wherein, Chelator₂ is a chelating group or a chelator structure for radionuclides, selected from the group consisting of: 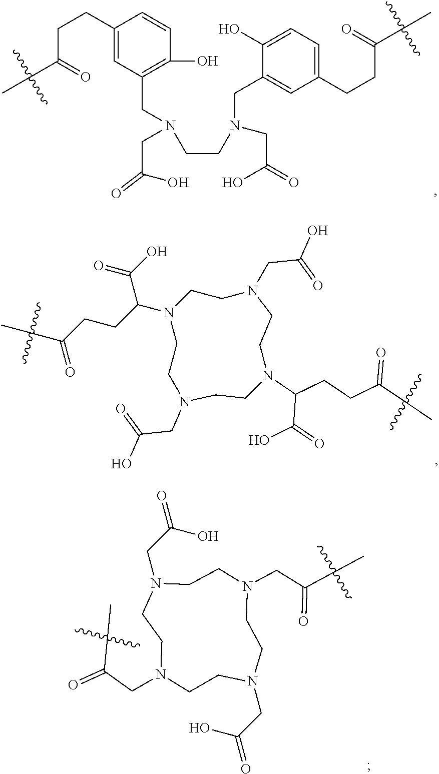 [SMILES: *C(=O)CCc1ccc(O)c(CN(CCN(CC(=O)O)Cc2cc(CCC(*)=O)ccc2O)CC(=O)O)c1 ; *C(=O)CCC(C(=O)O)N1CCN(CC(=O)O)CCN(C(CCC(*)=O)C(=O)O)CCN(CC(=O)O)CC1 ; *C(=O)CN1CCN(CC(=O)O)CCN(CC(*)=O)CCN(CC(=O)O)CC1]
R₂ is a nitroaromatic heterocyclic or nitrophenyl group selected from the group consisting of: 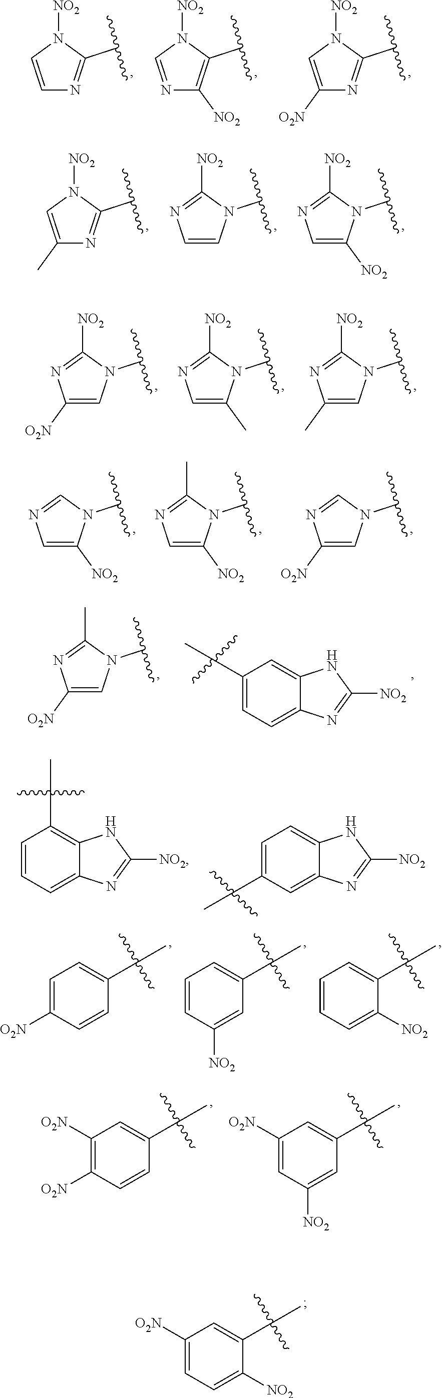
L₃ is a linker group between the Chelator₂ and a PSMA-targeted group, selected from the group consisting of: 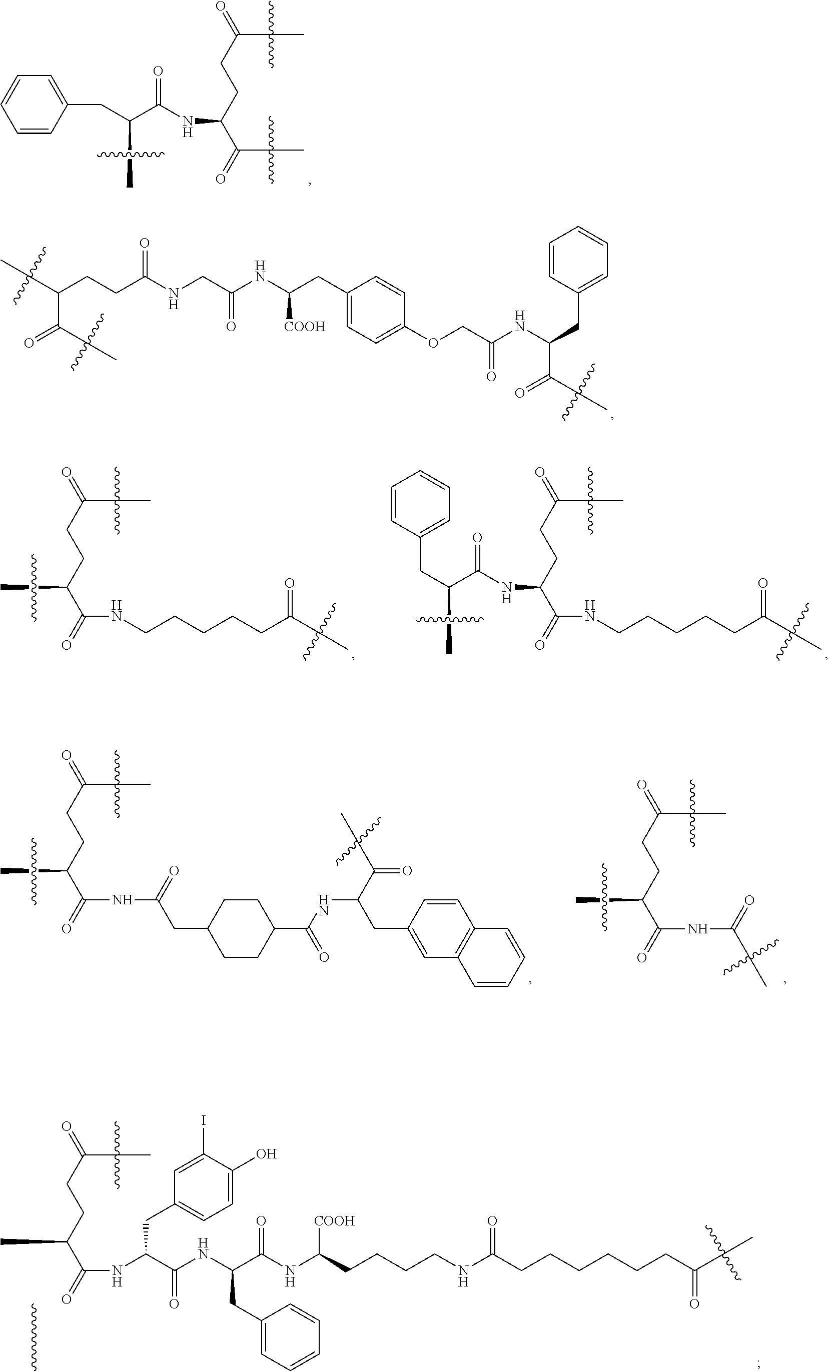
L₄ is a linker group between the Chelator₂ and the R₂, selected from the group consisting of: 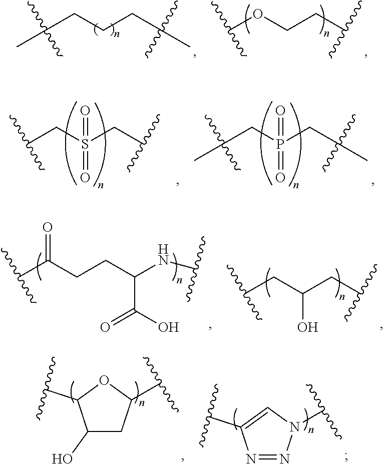
wherein n is an integer in a range from 0 to 6.

3. A prostate-specific membrane antigen (PSMA)-targeted radioactive metal complex containing a nitroaromatic heterocyclic group, represented by Formula I-2: 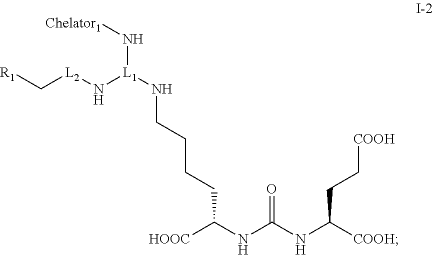
wherein, Chelator₁ is a chelator structure for radionuclides, selected from the group consisting of: 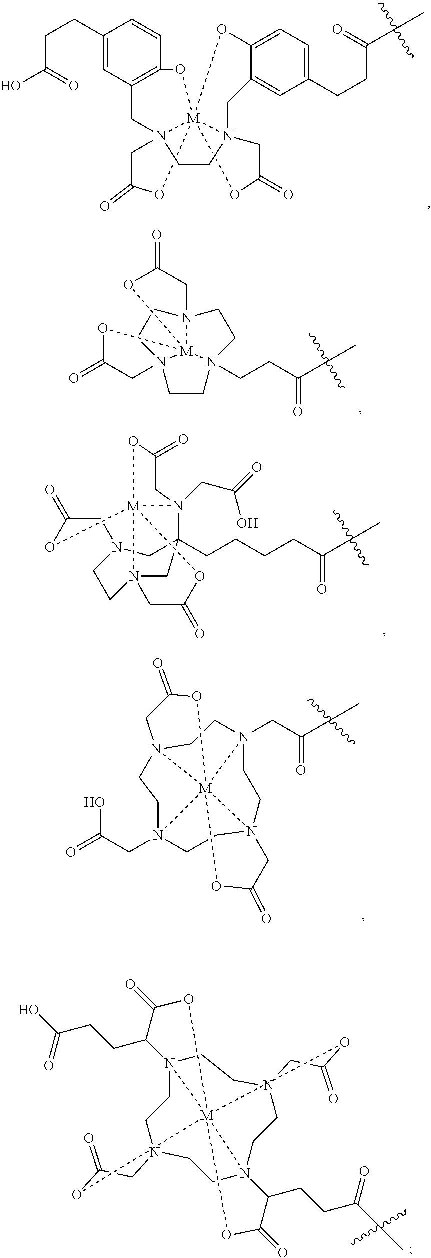
M is selected from the group comprising: ⁶⁸Ga, ¹⁸F—AlF, ¹⁷⁷Lu, ⁹⁰Y, Sc, ²²⁵Ac, ²¹²Pb, ⁶⁴Cu, ¹⁶¹Tb, and ²¹³Bi;
R₁ is a nitroaromatic heterocyclic or nitrophenyl group selected from the group consisting of: 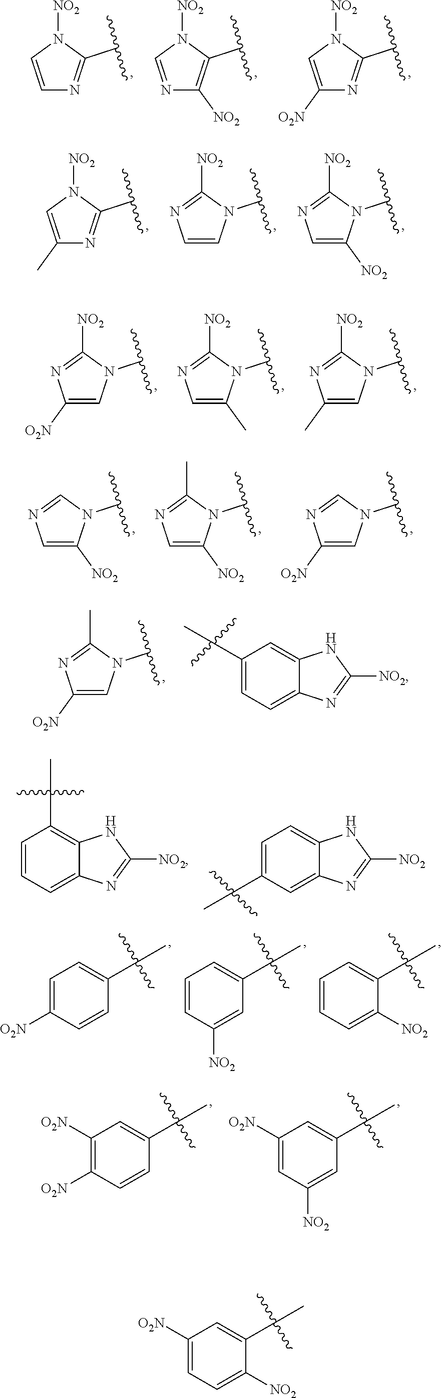
L₁ is a linker group between the Chelator₁ and a PSMA-targeted group, selected from the group consisting of: 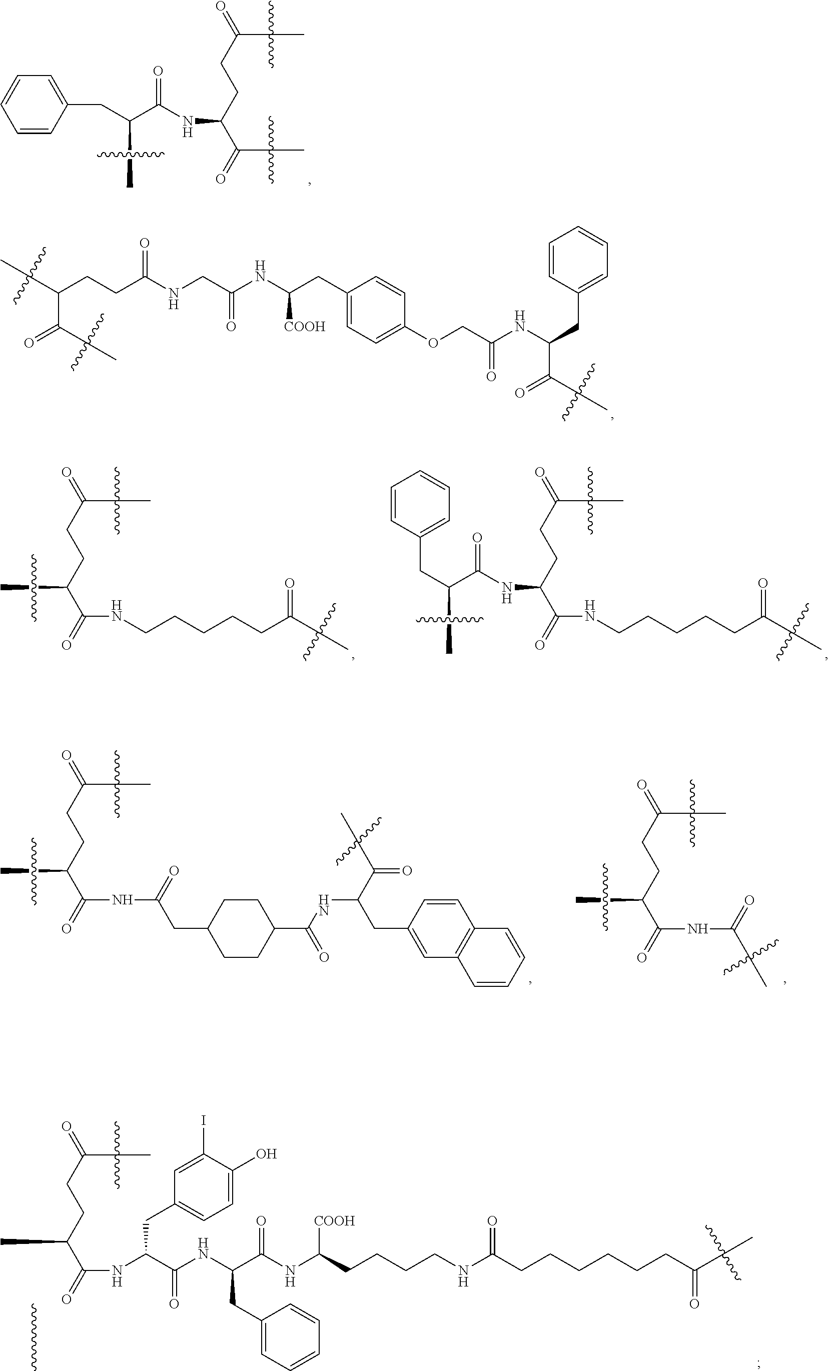
L₂ is a linker group between the Chelator₁ and the R₁, selected from the group consisting of: 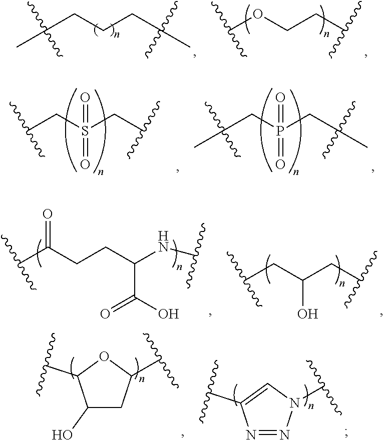
wherein n is an integer in a range from 0 to 6.

4. A prostate-specific membrane antigen (PSMA)-targeted radioactive metal complex containing a nitroaromatic heterocyclic group, represented by Formula II-1: 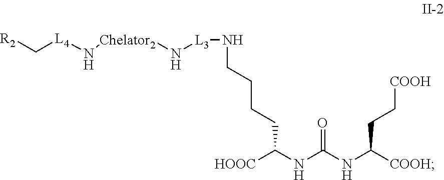
wherein, Chelator₂ is a chelating group or a chelator structure for radionuclides, selected from the group consisting of: 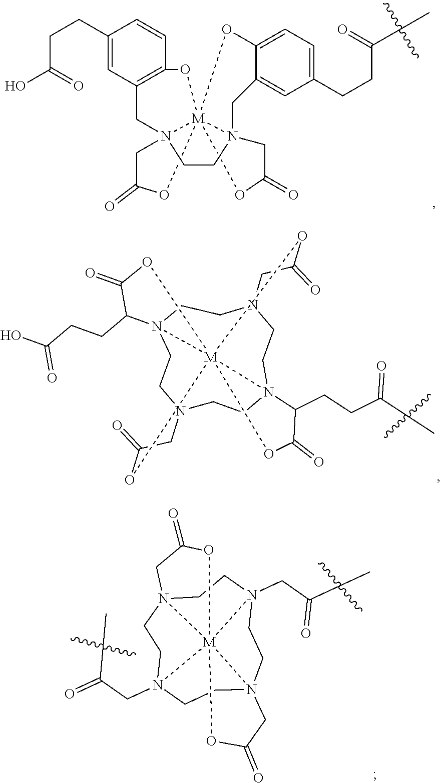
M is selected from the group comprising: ⁶⁸Ga, ¹⁸F—AlF, ¹⁷⁷Lu, ⁹⁰Y, Sc, ²²⁵Ac, ²¹²Pb, ⁶⁴Cu, ⁶¹Tb, and ²¹³Bi;
R₂ is a nitroaromatic heterocyclic or nitrophenyl group selected from the group consisting of: 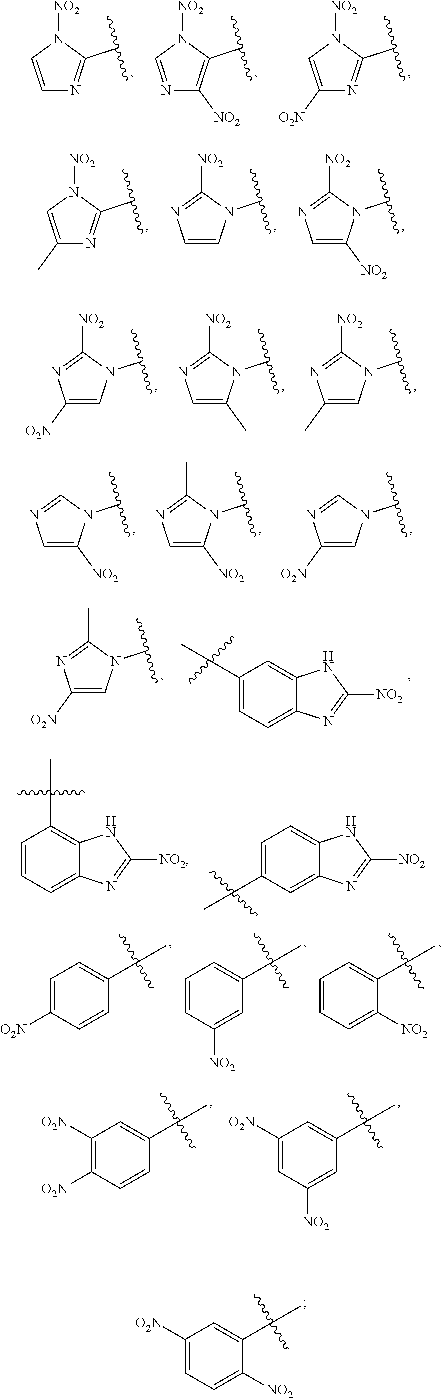
L₃ is a linker group between the Chelator₂ and a PSMA-targeted group, selected from the group consisting of: 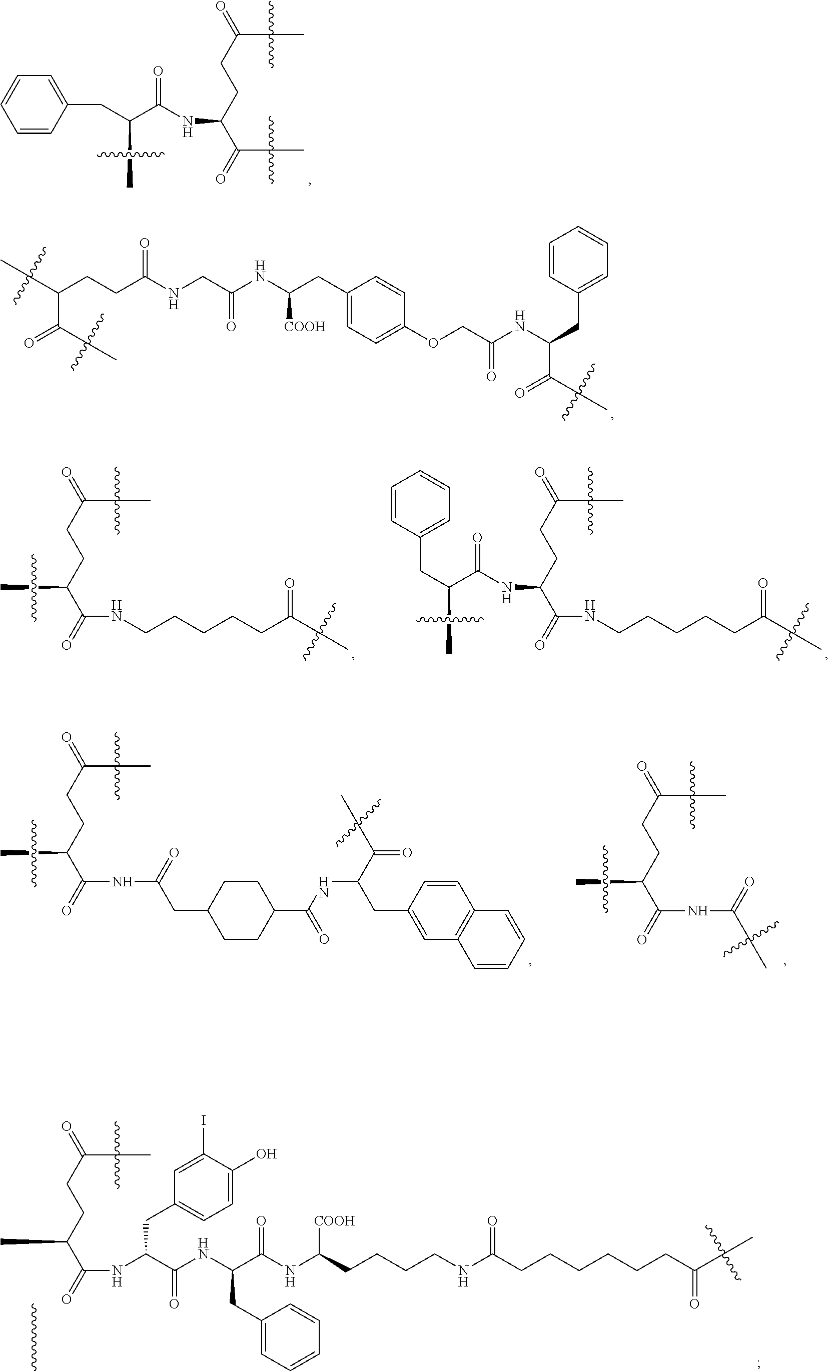
L₄ is a linker group between the Chelator₂ and the R₂, selected from the group consisting of: 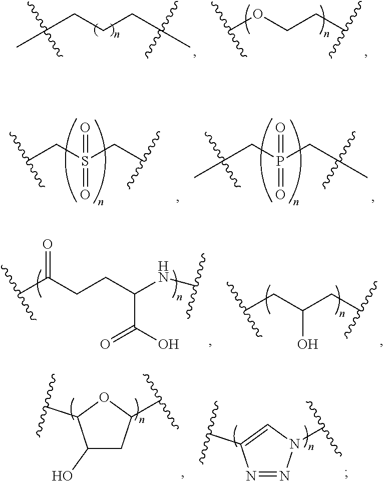
wherein n is an integer in a range from 0 to 6.

5. The PSMA-targeted radioactive metal ligand according to claim 1 or 2, having one of the following structure:

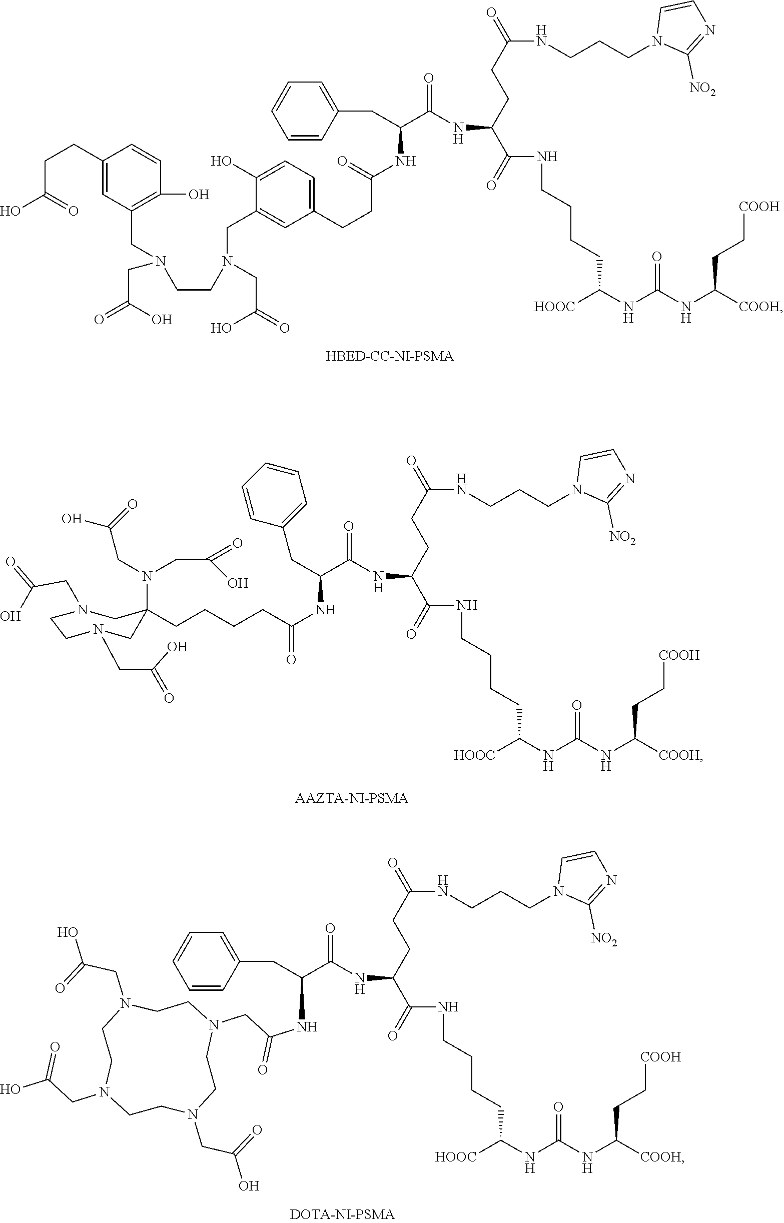

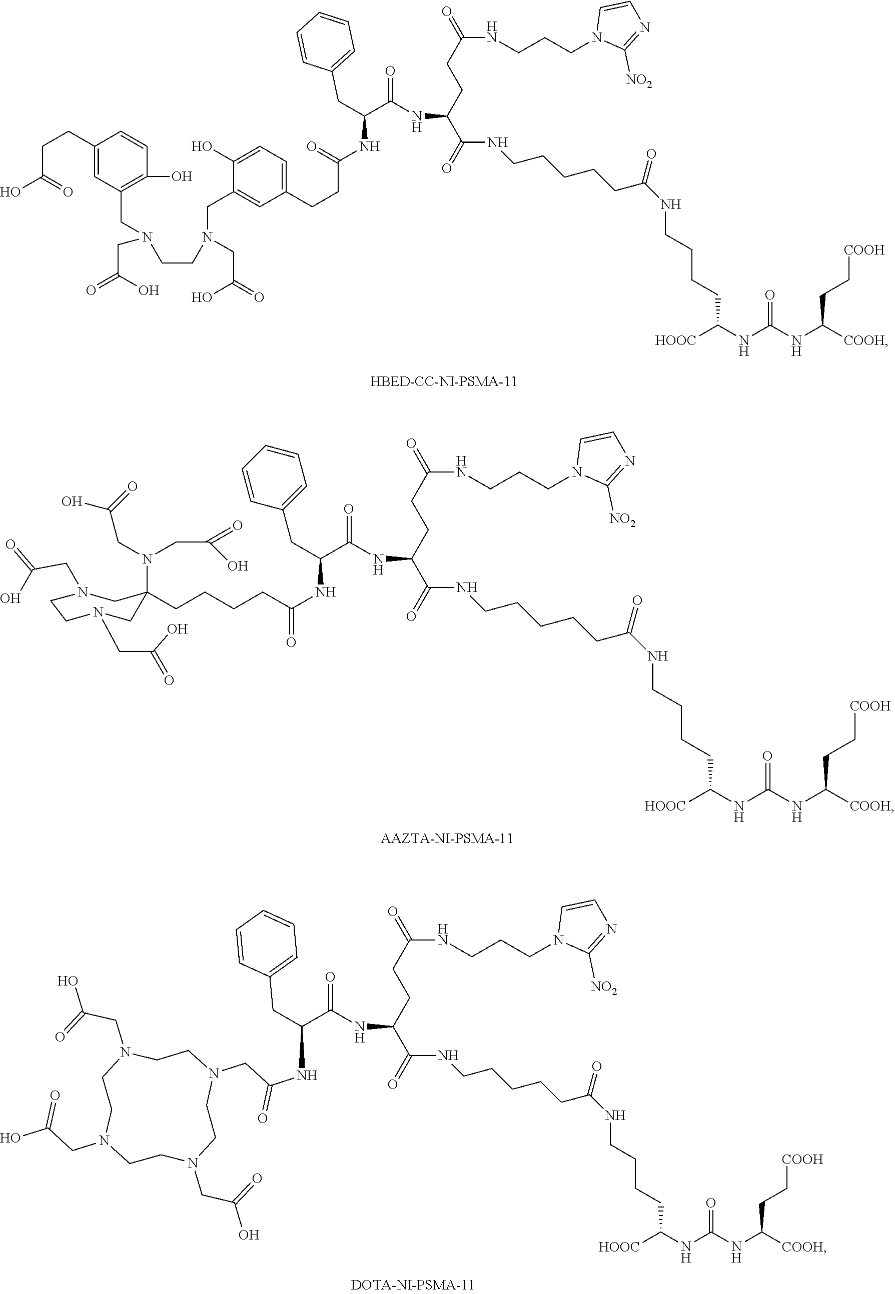

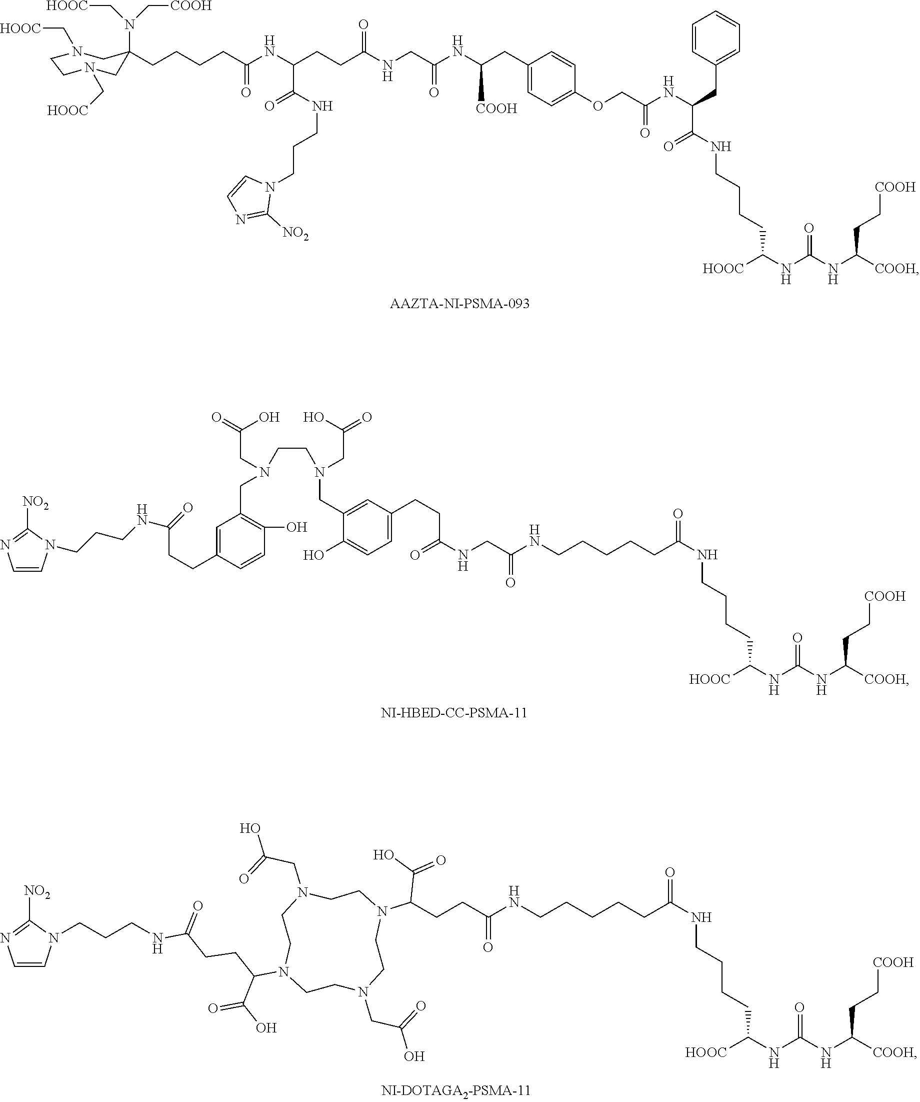

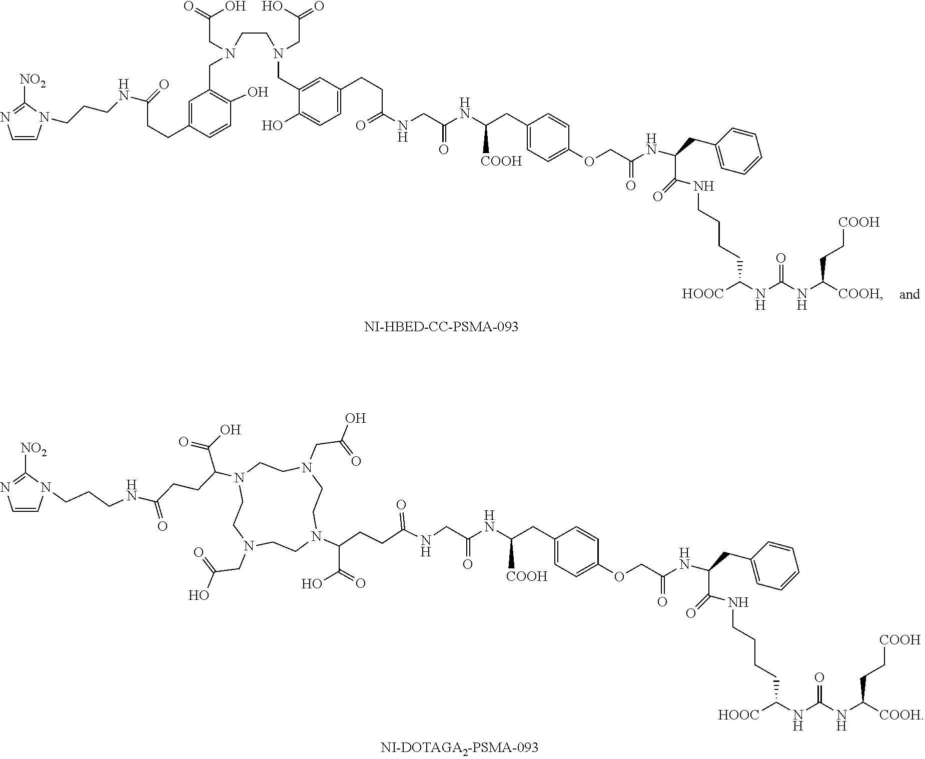 [SMILES: O=C(O)CC[C@H](NC(=O)N[C@@H](CCCCNC(=O)[C@H](Cc1ccccc1)NC(=O)COc1ccc(C[C@H](NC(=O)CNC(=O)CCc2ccc(O)c(CN(CCN(CC(=O)O)Cc3cc(CCC(=O)NCCCn4ccnc4[N+](=O)[O-])ccc3O)CC(=O)O)c2)C(=O)O)cc1)C(=O)O)C(=O)O ; O=C(O)CC[C@H](NC(=O)N[C@@H](CCCCNC(=O)[C@H](Cc1ccccc1)NC(=O)COc1ccc(C[C@H](NC(=O)CNC(=O)CCC(C(=O)O)N2CCN(CC(=O)O)CCN(C(CCC(=O)NCCCn3ccnc3[N+](=O)[O-])C(=O)O)CCN(CC(=O)O)CC2)C(=O)O)cc1)C(=O)O)C(=O)O]

6. The PSMA-targeted radioactive metal complex according to claim 3 or 4, having one of the following structure:

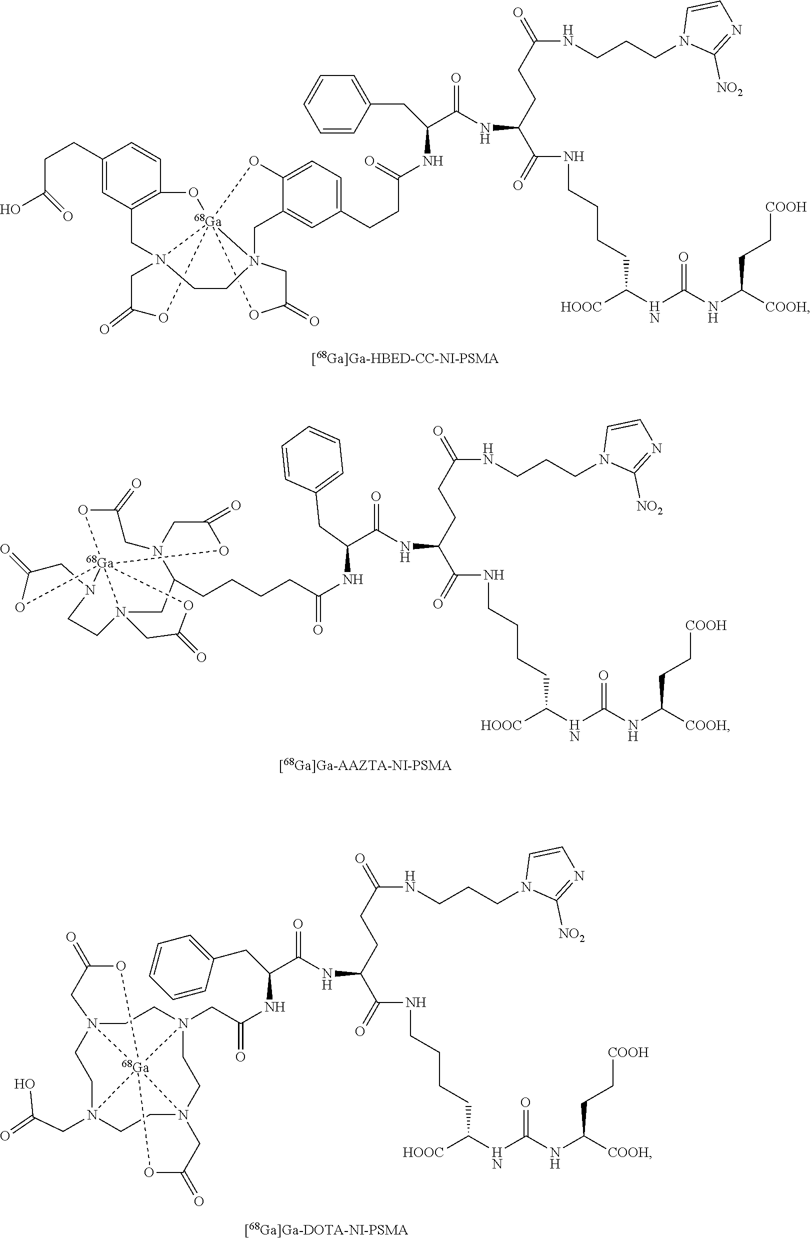

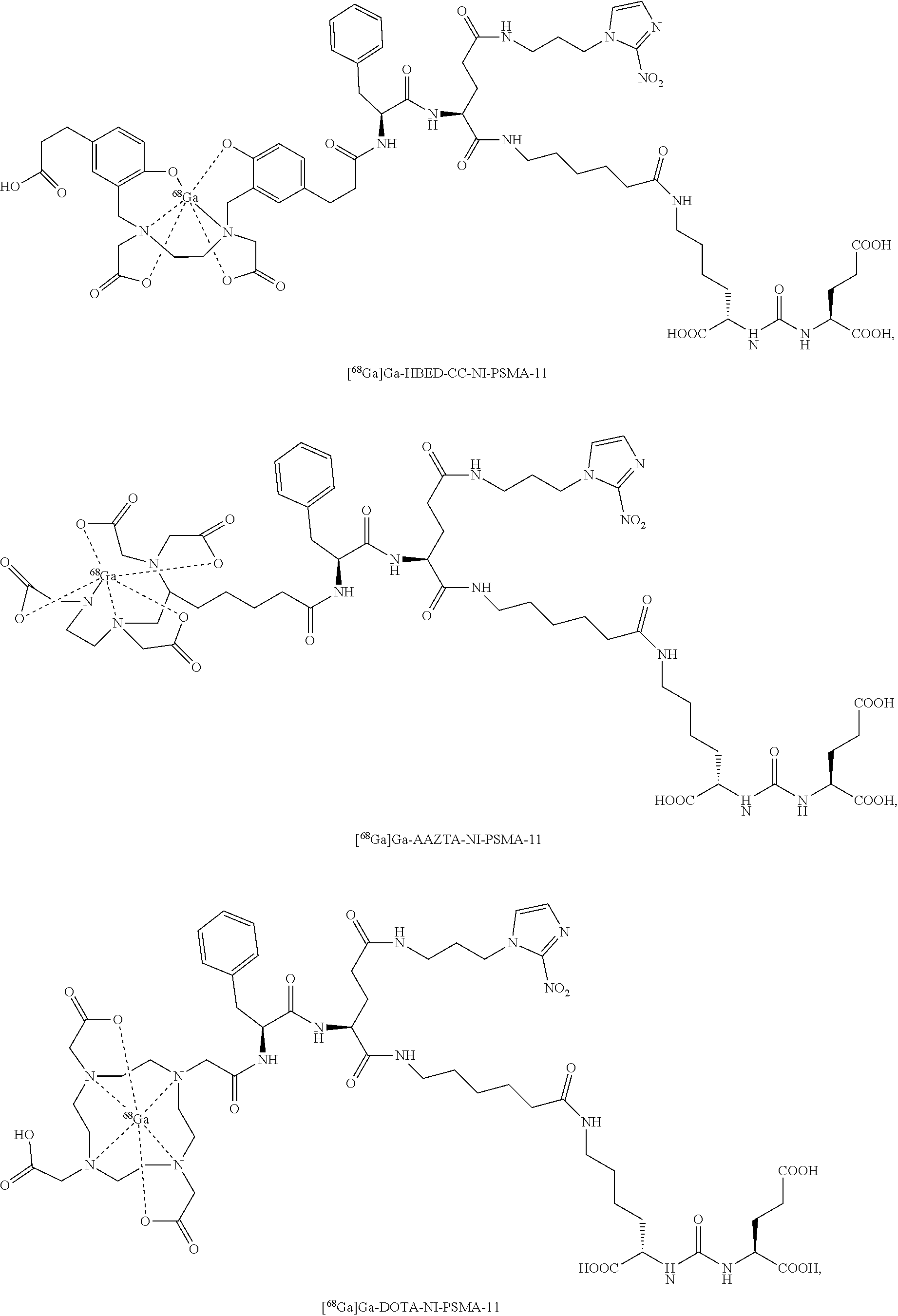

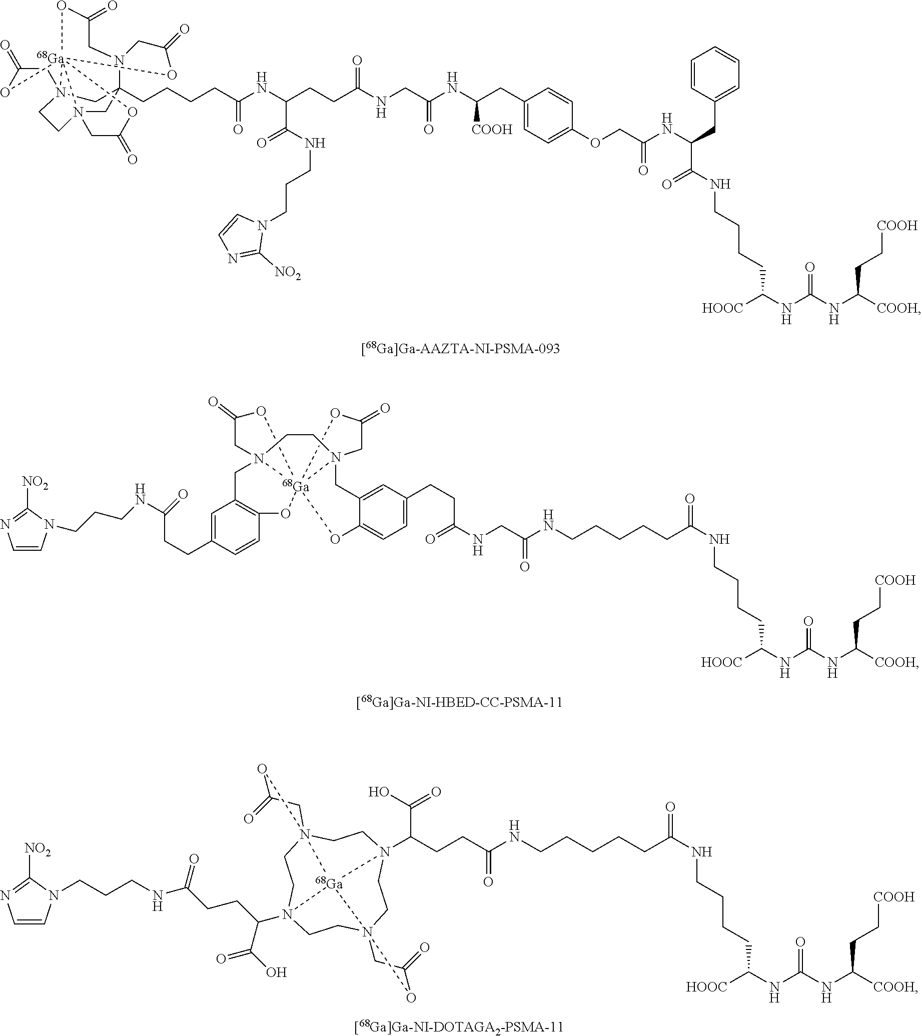

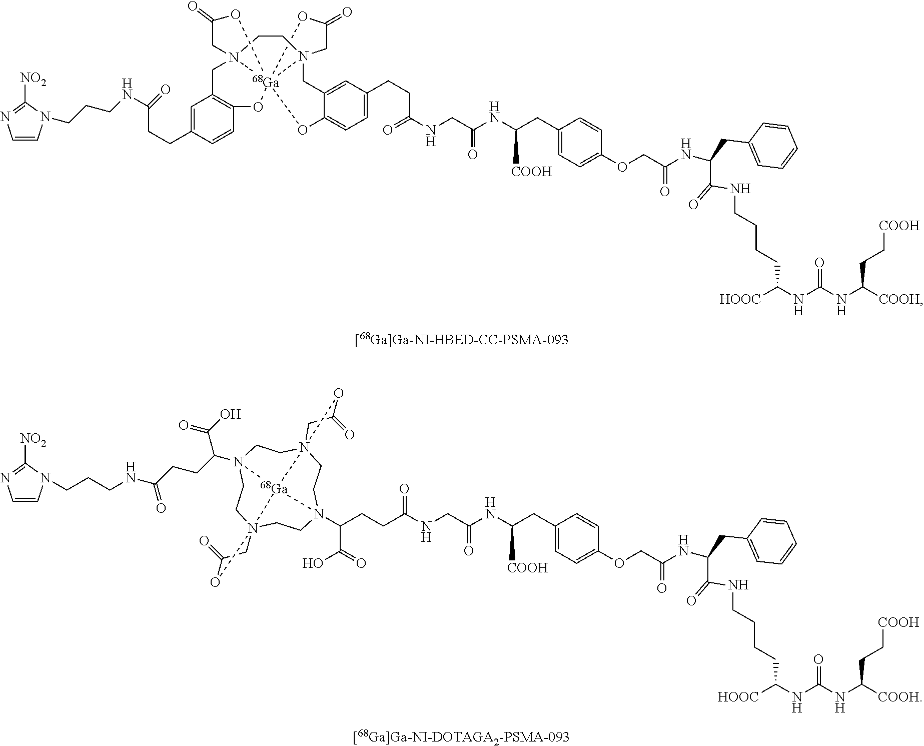

7. A preparation method of the PSMA-targeted radioactive metal ligand according to claim 1, comprising:
(1) dissolving triphosgene in dichloromethane to form a solution; slowly adding a solution of N(ε)-carbobenzyloxy-L-lysine tert-butyl ester hydrochloride (H-Lys(Z)-Ot-Bu·HCl) and triethylamine in dichloromethane dropwise to the solution, and then slowly adding a solution of L-glutamic acid di-tert-butyl ester hydrochloride and triethylamine in dichloromethane dropwise to form a reaction mixture; stirring the reaction mixture at room temperature, distilling under reduced pressure, and purifying by silica gel column chromatography where a volume ratio of petroleum ether/ethyl acetate is 1/1 to obtain a colorless oily product; dissolving the colorless oily product in tetrahydrofuran, adding 10% Pd/C, and stirring under H atmosphere at room temperature to form a reaction solution; filtering the reaction solution through Celite, and performing evaporation under reduced pressure to remove solvent to obtain brown oily compound Lys(t-Bu)-CO-Glu(t-Bu); dissolving Lys(t-Bu)-CO-Glu(t-Bu) and Cbz-L-NH in anhydrous DMF, and adding 2-(7-azabenzotriazol-1-yl)-N,N,N′,N′-tetramethyluronium hexafluorophosphate (HATU) and N,N′-diisopropylethylamine (DIPEA) under an ice bath for reaction at room temperature overnight to obtain a reaction product; isolating and purifying the reaction product to obtain pale-yellow oily compound; dissolving the pale-yellow oily compound in methanol, adding Pd/C powder for reduction reaction under H atmosphere overnight to obtain NH-L-Lys(t-Bu)-CO-Glu(t-Bu);
(2) dissolving (S)-4-(((9H-fluoren-9-yl)methoxy)carbonyl)amino)-5-(tert-butoxy)-5-oxopentanoic acid in anhydrous DMF, and adding HATU and DIPEA under an ice bath, stirring, then adding R-L-NH to obtain a mixture; stirring the mixture at room temperature overnight to obtain another reaction mixture, and washing the reaction mixture with ethyl acetate and saturated brine; collecting organic phase and drying the organic phase with anhydrous Na SO, filtering and removing the anhydrous Na SO, and performing evaporation under reduced pressure; after solvent removal, performing purification by silica gel column chromatography where a volume ratio of dichloromethane/methanol/ammonia water is 25/1/0.1 to obtain a pale-yellow solid product; dissolving the pale-yellow solid product in trifluoroacetic acid, stirring at room temperature, distilling under reduced pressure to obtain pale-yellow solid compound; dissolving the pale-yellow solid compound in anhydrous DMF, adding HATU and DIPEA under an ice bath, stirring, and adding a solution of NH-L-Lys(t-Bu)-CO-Glu(t-Bu) in anhydrous DMF; leaving for reaction at room temperature overnight to obtain another reaction product, washing the reaction product with ethyl acetate and saturated brine, and collecting organic phase and drying the organic phase with anhydrous Na SO, then filtering and removing the anhydrous Na SO to obtain filtrate; performing evaporation on the filtrate under reduced pressure and removing solvent to obtain residue; purifying the residue by flash chromatography where a volume ratio of dichloromethane/methanol/ammonia water is 15/1/0.1 to obtain another pale-yellow solid compound, dissolving the pale-yellow solid compound in dichloromethane, adding diethylamine dropwise, stirring at room temperature, and performing evaporation under reduced pressure to remove solvent to obtain R-L-NH-L-Lys (t-Bu)-CO-Glu (t-Bu);
(3) dissolving chelator HBED-CC, AAZTA, DOTA, or NOTA in anhydrous DMF, and adding HATU and DIPEA under an ice bath, stirring, then adding the R-L-NH-L-Lys(t-Bu)-CO-Glu(t-Bu) from step (2); stirring at room temperature overnight, purifying by silica gel column chromatography to obtain pale-yellow oily liquid R-L-NH-L (chelator1)-Lys(t-Bu)-CO-Glu(t-Bu); dissolving the pale-yellow oily liquid in trifluoroacetic acid, stirring at room temperature, distilling under reduced pressure to remove solvent, and purifying by semi-preparative HPLC to obtain the R-L-NH-L (chelator1)-Lys-CO-Glu represented by the Formula I-1.

8. A preparation method of the PSMA-targeted radioactive metal ligand according to claim 2, comprising:
(1) dissolving triphosgene in dichloromethane to form a solution; slowly adding a solution of N(ε)-carbobenzyloxy-L-lysine tert-butyl ester hydrochloride (H-Lys(Z)-Ot-Bu·HCl) and triethylamine in dichloromethane dropwise to the solution, and then slowly adding a solution of L-glutamic acid di-tert-butyl ester hydrochloride and triethylamine in dichloromethane dropwise to form a reaction mixture; stirring the reaction mixture at room temperature, distilling under reduced pressure, and purifying by silica gel column chromatography where a volume ratio of petroleum ether/ethyl acetate is 1/1 to obtain a colorless oily product; dissolving the colorless oily product in tetrahydrofuran, adding 10% Pd/C, and stirring under H atmosphere at room temperature to form a reaction solution; filtering the reaction solution through Celite, and performing evaporation under reduced pressure to remove solvent to obtain brown oily compound Lys(t-Bu)-CO-Glu(t-Bu); dissolving Lys(t-Bu)-CO-Glu(t-Bu) and Cbz-L₃-NH in anhydrous DMF, and adding 2-(7-azabenzotriazol-1-yl)-N,N,N′,N′-tetramethyluronium hexafluorophosphate (HATU) and N,N′-diisopropylethylamine (DIPEA) under an ice bath for reaction at room temperature overnight to obtain a reaction product; isolating and purifying the reaction product to obtain pale-yellow oily compound; dissolving the pale-yellow oily compound in methanol, adding Pd/C powder for reduction reaction under H atmosphere overnight to obtain NH-L₃-Lys (t-Bu)-CO-Glu (t-Bu);
(2) dissolving chelator HBED-CC, AAZTA, DOTA, or NOTA in anhydrous DMF, and adding HATU and DIPEA under an ice bath, stirring, then adding R-L-NH; stirting at room temperature overnight, washing with ethyl acetate and saturated brine; collecting organic phase and drying the organic phase with anhydrous Na SO, filtering and removing the anhydrous Na SO, and performing evaporation under reduced pressure; after solvent removal, performing purification by silica gel column chromatography where a volume ratio of dichloromethane/methanol/ammonia water is 90/10/0.1 to obtain pale-yellow solid R-L-NH-chelator₂; dissolving the pale-yellow solid in anhydrous DMF, adding HATU and DIPEA under an ice bath, stirring, adding the NH-L-Lys(t-Bu)-CO-Glu(t-Bu) from step (1), and further adding R-L-NH; stirring at room temperature overnight to obtain another reaction product, washing the reaction product with ethyl acetate and saturated brine, and collecting organic phase and drying the organic phase with anhydrous Na SO, then filtering and removing the anhydrous Na SO to obtain filtrate; performing evaporation on the filtrate under reduced pressure and removing solvent to obtain residue; purifying the residue by silica gel column chromatography where a volume ratio of dichloromethane/methanol/ammonia water is 90/10/0.1 to obtain oily liquid R-L-NH-chelator₂-NH-L-Lys (t-Bu)-CO-Glu(t-Bu); dissolving the oily liquid in trifluoroacetic acid, stirring at room temperature, distilling under reduced pressure to remove solvent, and purifying by semi-preparative HPLC to obtain the R-L-NH-chelator₂-NH-L-Lys-CO-Glu represented by the Formula II-1.

9. A preparation method of the PSMA-targeted radioactive metal complex according to claim 3, comprising:
based on claim 7, adding the following: dissolving the R-L-NH-L (chelator₁)-Lys-CO-Glu from step (3) in sodium acetate buffer; adding a solution of [⁶⁸Ga]GaCl₃ or [¹⁷⁷Lu]LuCl₃ containing radionuclides; and reacting under heating for 5-15 minutes to obtain the radioactive metal complex represented by the Formula I-2.

10. A preparation method of the PSMA-targeted radioactive metal complex according to claim 4, comprising:
based on claim 8, adding the following: dissolving the R-L-NH-chelator₂-NH-L-Lys-CO-Glu from step (2) in sodium acetate buffer; adding a solution of [⁶⁸Ga]GaCl₃ or [¹⁷⁷Lu]LuCl₁ containing radionuclides; and reacting under heating for 5-15 minutes to obtain the radioactive metal complex represented by the Formula II-2.
